(19) United States
(12) Patent Application Publication
(10) Pub. No.: US 2024/0182509 A1
(43) Pub. Date: Jun. 6, 2024

(54) BICYCLIC HETEROCYCLIC COMPOUNDS FOR PROPHYLAXIS AND TREATMENT OF VIRAL INFECTIONS

(71) Applicant: BM Pharma Consulting Pty Ltd., Parrearra (AU)

(72) Inventors: Brett Antony Mooney, Parrearra (AU); Rangachari Narasimhan, Sunnyvale, CA (US)

(21) Appl. No.: 18/506,847

(22) Filed: Nov. 10, 2023

Related U.S. Application Data
(63) Continuation-in-part of application No. PCT/IB2022/054130, filed on May 4, 2022.
(60) Provisional application No. 63/188,779, filed on May 14, 2021.

(51) Int. Cl. C07H 19/056; A61K 31/706

(52) U.S. Cl. CPC C07H 19/056; A61K 31/706

(57) ABSTRACT

Bicyclic heterocyclic compounds and derivatives thereof that have antiviral properties are disclosed. Pharmaceutical compositions and pharmaceutical formulations in unit dosage form suitable for the delivery of the compounds to a subject in need thereof are disclosed. The pharmaceutical compositions or formulations may include one or more active agents in addition to the compounds, such as one or more antiviral agents and/or one or more anti-inflammatory agents. Methods for preventing, treating, or ameliorating one or more symptoms associated with a viral infection in a subject are also disclosed. The methods include (i) administering to a subject an effective amount of the compound(s) to prevent, treat, or ameliorate one or more symptoms associated with a viral infection in the subject. The compound(s) can be administered by oral administration, parenteral administration, inhalation, mucosal administration, or a combination thereof.

### CROSS-REFERENCE TO RELATED APPLICATIONS

This application is a continuation-in-part of PCT/IB2022/054130 filed under the Patent Cooperation Treaty on May 4, 2022, which claims the benefit of and priority to U.S. Provisional Application No. 63/188,779 filed May 14, 2021, the entire contents of which are incorporated herein by reference for all purpose in their entirety.

### FIELD OF THE INVENTION

The invention is generally in the field of bicyclic heterocyclic compounds and their derivatives and methods for using them.

### BACKGROUND OF THE INVENTION

The first antiviral agent dates back to Idoxuridine which was approved in 1962 and is a pyrimidine-based compound. Ribavirin was approved in the early 1970s as a broad-spectrum antiviral agent and are still being used 50 years later. The discovery of acyclovir in 1978 was significant in the evolution of antiviral agents. This further improvement from the acyclovir arena namely Adefovir, Tenofovir, and Sofosbuvir provided a treatment primarily against the herpes viruses.

Favipiravir and ribavirin, both antivirals, are RNA polymerase inhibitors, metabolized intracellularly into their respective active forms of ribosyl triphosphate that inhibit the viral RNA polymerase and induce lethal mutagenesis. It has been shown that the favipiravir-RTP mimics guanosine triphosphate (GTP) and adenosine triphosphate (ATP) and causes lethal mutagenesis. Ribavirin has been shown to induce mutagenesis in the influenza virus genome by acting as an ambiguous pairing analogue and increasing G-to-A and C-to-U mutations.

The ambiguous base pairing behaviour of both ribavirin and favipiravir has been attributed to the rotating carboxamide (Stevaert et al., Medicinal Research Reviews, 36(6):1127-1173 (2016)). A carboxamide can also exist as an ionized base pair as a tautomer. In the search for an antiviral for dengue virus, it was reported by Qiu et al. that ambiguous base pairing originates from rotational or tautomeric forms of the base that result in the mutagenic nucleoside triphosphate, resembling more than one natural nucleoside triphosphate (Qiu et al., PLOS Neglected Tropical Diseases, 12(4):e0006421 (2018)).

Ribavirin has side effects that resulted in a boxed warning by the Food and Drug Administration (“FDA”). The product is administered as either a 200 mg, 400 mg, or 600 mg tablet with maximum dose of 1,200 mg per day over 48 weeks. Favipiravir is currently approved in Japan and is sold as a 200 mg tablet with posology of 1,600 mg orally twice daily for 1 day followed by 600 mg twice daily for 4 days and is indicated for influenza. Its in vitro activity against COVID-19 prompted its experimental use against this endemic and is now approved for use in treatment of early onset SARS-CoV2 in Japan, China, and Italy. Favipiravir is also teratogenic with a red box warning on the PI relating to its teratogenic effects in Japan.

There remains a need to develop compounds that possess antiviral properties and low toxicity.

Therefore, it is the object of the present invention to provide compounds that possess antiviral properties and low toxicity.

It is another object of the present invention to provide methods of using such compounds.

### SUMMARY OF THE INVENTION

Compounds and methods for preventing, treating, or ameliorating one or more symptoms associated with a viral infection in a subject have been developed. The compounds are bicyclic heterocyclic compounds and derivatives thereof. Generally, the compounds can mimic RNA and/or DNA bases through base pairing with RNA and/or DNA nucleosides, thereby being incorporated into the viral RNA and/or DNA strand, resulting in viral mutagenesis. The compound can be a nucleoside or a nucleotide. Typically, the compounds can form at least two hydrogen bonds with RNA and/or DNA nucleosides and eliminate free rotation of groups involved in the hydrogen bonding. In some embodiments, the compounds have the structures of any one of Formulae I-XXII and 1-102.

Pharmaceutical compositions and pharmaceutical formulations in unit dosage form suitable for the delivery of the compounds and their preparation have been developed. Generally, the pharmaceutical composition or formulation contains the compound(s) and a pharmaceutically acceptable excipient. The compound(s) in the pharmaceutical compositions or formulations is in an effective amount for preventing, treating, or ameliorating one or more symptoms associated with a viral infection in a subject. In some embodiments, the pharmaceutical composition or formulation can further contain one or more active agents in addition to the compounds, such as one or more antiviral agents and/or one or more anti-inflammatory agents.

The methods include (i) administering to a subject an effective amount of the compound(s) to prevent, treat, or ameliorate one or more symptoms associated with a viral infection in the subject. The subject is typically a mammal, preferably a human. The compound(s) can be administered by oral administration, parenteral administration, inhalation, mucosal, topical administration, or a combination thereof. In some embodiments, the methods can further include administering to the subject a second active agent, such as an antiviral agent or an anti-inflammatory agent, optionally more than one second active agent, such as one or more antiviral agents and/or one or more anti-inflammatory agents, prior to, during, and/or subsequent to administration of the first compound.

The compounds have broad antiviral properties and are suitable for use in the prophylaxis and/or treatment of all classes of viruses. For example, the compounds can be administered to a subject for preventing, treating, or ameliorating one or more symptoms associated with a viral infection caused by double stranded DNA viruses, single stranded DNA viruses, double stranded RNA viruses, single stranded positive sense RNA viruses, single stranded negative sense RNA viruses, positive sense single stranded RNA viruses that replicate through a DNA intermediate, double stranded DNA viruses that replicate through a single stranded RNA intermediate, single stranded RNA viruses with reverse transcriptase, or double stranded DNA viruses with reverse transcriptase, or a combination thereof.

### BRIEF DESCRIPTION OF THE DRAWINGS

FIG. 1A is a graph illustrating the hydrogen bonding between the enol form of a triazolo-pyrazine compound and a nucleoside, cytidine. FIG. 1B is a graph illustrating the hydrogen bonding between the enol form of a triazolo-pyrazine compound and an imino form of the nucleoside, cytidine.

FIG. 2A is a graph illustrating the hydrogen bonding between a triazolo-pyrazine compound and the enol form of a nucleoside, uridine. FIG. 2B is a graph illustrating the hydrogen bonding between a triazolo-pyrazine compound and the keto form of a nucleoside, uridine.

FIGS. 3A-3C are graphs illustrating bonding between the amino-keto active RTP metabolite of the compound of Formula 84 and Cytidine Monophosphate within the SARS-CoV-2 RNA dependent RNA polymerase (RdRp) (FIG. 3A), bonding between reference compound Guanosine TP and Cytidine Monophosphate within the SARS-CoV-2 RdRp (FIG. 3B), and bonding between reference compound Bemnifosbuvir (data generated using the active metabolite, i.e., AT-9010) and Cytidine Monophosphate within the SARS-CoV-2 RdRp (FIG. 3C). The three hydrogen bonds between the nucleoside TP and cytidine MP (C, T:101), as indicated by three arrows, show Watson Crick base pairing.

FIGS. 4A-4C are graphs illustrating bonding between the amino-enol active RTP metabolite of the compound of Formula 84 and Uridine MP within the SARS-CoV-2 RdRp (FIG. 4A), bonding between reference compound Adenosine and Uridine MP within the SARS-CoV-2 RdRp (FIG. 4B), and bonding between reference compound Remdesivir (active metabolite as TP) and Uridine MP within the SARS-CoV-2 RdRp (FIG. 4C).

FIGS. 5A-5B are graphs illustrating bonding between the keto-enol active RTP metabolite of the compound of Formula 102 and Cytidine MP within the SARS-CoV-2 RdRp (FIG. 5A), and bonding between the enol-enol active RTP metabolite of the compound of Formula 102 and Uridine MP within the SARS-CoV-2 RdRp (FIG. 5B).

### DETAILED DESCRIPTION OF THE INVENTION

### I. Definitions

As used herein, the term “aliphatic” refers to hydrocarbon groups that do not contain conjugated pi-systems that obey Hickel's rule. Aliphatic groups represent saturated hydrocarbons and unsaturated hydrocarbons, including those that are linear, branched, or cyclic (either monocyclic or polycyclic). An aliphatic group can be a linear C₁-C₃₀ aliphatic, a branched C₄-C₃₀ aliphatic, a cyclic C₃-C₃₀ aliphatic, a linear C₁-C₃₀ aliphatic or a branched C₄-C₃₀ aliphatic, a linear C₁-C₃₀ aliphatic or a cyclic C₃-C₃₀ aliphatic, a branched C₄-C₃₀ aliphatic or a cyclic C₃-C₃₀ aliphatic. Optionally, aliphatic groups have up to 20 carbon atoms. An aliphatic can be a linear C₁-C₂₀ aliphatic, a branched C₄-C₂₀ aliphatic, a cyclic C₃-C₂₀ aliphatic, a linear C₁-C₂₀ aliphatic or a branched C₄-C₂₀ aliphatic, a branched C₄-C₂₀ aliphatic or a cyclic C₃-C₂₀ aliphatic, a linear C₁-C₂₀ aliphatic or a cyclic C₃-C₂₀ aliphatic. Optionally, aliphatic groups have up to 14 carbon atoms. An aliphatic can be a linear C₁-C₁₄ aliphatic, a branched C₄-C₁₄ aliphatic, a cyclic C₃-C₁₄ aliphatic, a linear C₁-C₁₄ aliphatic or a branched C₄-C₁₄ aliphatic, a branched C₄-C₁₄ aliphatic or a cyclic C₃-C₁₄ aliphatic, a linear C₁-C₁₄ aliphatic or a cyclic C₃-C₁₄ aliphatic. An aliphatic can be a linear C₁-C₁₀ aliphatic, a branched C₄-C₁₀ aliphatic, a cyclic C₃-C₁₀ aliphatic, a linear C₁-C₁₀ aliphatic or a branched C₄-C₁₀ aliphatic, a branched C₄-C₁₀ aliphatic or a cyclic C₃-C₁₀ aliphatic, a linear C₁-C₁₀ aliphatic or a cyclic C₃-C₁₀ aliphatic. Optionally, aliphatic groups have up to 6 carbon atoms. An aliphatic can be a linear C₁-C₆ aliphatic, a branched C₄-C₆ aliphatic, a cyclic C₃-C₆ aliphatic, a linear C₁-C₆ aliphatic or a branched C₄-C₆ aliphatic, a branched C₄-C₆ aliphatic or a cyclic C₃-C₆ aliphatic, or a linear C₁-C₆ aliphatic or a cyclic C₃-C₆ aliphatic. Optionally, aliphatic groups have up to four carbons. An aliphatic can be a linear C₁-C₄ aliphatic, a cyclic C₃-C₄ aliphatic, a linear C₁-C₄ aliphatic or a cyclic C₃-C₄ aliphatic.

As used herein, the term “heterocyclic” refers to a chain of carbon and heteroatoms, wherein the heteroatoms are selected from nitrogen, oxygen, and sulfur, at least a portion of which, including at least one heteroatom, form a ring.

As used herein, the term “amino” includes the group NH₂ (primary amino), alkylamino (secondary amino), and dialkylamino (tertiary amino), where the two alkyl groups in dialkylamino may be the same or different, i.e. alkylalkylamino.

Illustratively, amino include methylamino, ethylamino, dimethylamino, and methylethylamino. In addition, it is to be understood that when amino modifies or is modified by another term, such as aminoalkyl, or acylamino, the above variations of the term amino continue to apply. Illustratively, aminoalkyl includes H²N-alkyl, methylaminoalkyl, ethylaminoalkyl, dimethylaminoalkyl, methylethylaminoalkyl, and the like. Illustratively, acylamino includes acylmethylamino, and acylethylamino.

As used herein, the term “amide” includes the group CONH₂ (primary amide), CONHalkyl (secondary amide), and CONdialkyl (tertiary amide), where the two alkyl groups in CONdialkyl may be the same or different.

As used herein, the term “prodrug” generally refers to compounds that are labile in vivo under predetermined biological conditions.

As used herein, the term “effective amount” means a dosage sufficient to prevent, treat, or alleviate one or more symptoms of a disease state being treated or to otherwise provide a desired pharmacologic and/or physiologic effect. The precise dosage will vary according to a variety of factors such as subject-dependent variables (e.g., age, immune system health, etc.), the disease, and the treatment being administered.

As used herein, the term “pharmaceutically acceptable” means a non-toxic material that does not interfere with the effectiveness of the biological activity of the active ingredients.

As used herein, the term “alkyl” refers to univalent groups derived from alkanes by removal of a hydrogen atom from any carbon atom. Alkanes represent saturated hydrocarbons, including those that are linear, branched, or cyclic (either monocyclic or polycyclic). An alkyl can be a linear C₁-C₃₀ alkyl, a branched C₄-C₃₀ alkyl, a cyclic C₃-C₃₀ alkyl, a linear C₁-C₃₀ alkyl or a branched C₄-C₃₀ alkyl, a linear C₁-C₃₀ alkyl or a cyclic C₃-C₃₀ alkyl, a branched C₄-C₃₀ alkyl or a cyclic C₃-C₃₀ alkyl. Optionally, alkyl groups have up to 20 carbon atoms. An alkyl can be a linear C₁-C₂₀ alkyl, a branched C₄-C₂₀ alkyl, a cyclic C₃-C₂₀ alkyl, a linear C₁-C₂₀ alkyl or a branched C₄-C₂₀ alkyl, a branched C₄-C₂₀ alkyl or a cyclic C₃-C₂₀ alkyl, a linear C₁-C₂₀ alkyl or a cyclic C₃-C₂₀ alkyl. Optionally, alkyl groups have up to 10 carbon atoms. An alkyl can be a linear C₁-C₁₀ alkyl, a branched C₄-C₁₀ alkyl, a cyclic C₃-C₁₀ alkyl, a linear C₁-C₁₀ alkyl or a branched C₄-C₁₀ alkyl, a branched C₄-C₁₀ alkyl or a cyclic C₃-C₁₀ alkyl, a linear C₁-C₁₀ alkyl or a cyclic C₃-C₁₀ alkyl. Optionally, alkyl groups have up to 6 carbon atoms. An alkyl can be a linear C₁-C₆ alkyl, a branched C₄-C₆ alkyl, a cyclic C₃-C₆ alkyl, a linear C₁-C₆ alkyl or a branched C₄-C₆ alkyl, a branched C₄-C₆ alkyl or a cyclic C₃-C₆ alkyl, or a linear C₁-C₆ alkyl or a cyclic C₃-C₆ alkyl. Optionally, alkyl groups have up to four carbons. An alkyl can be a linear C₁-C₄ alkyl, cyclic C₃-C₄ alkyl, a linear C₁-C₄ alkyl or a cyclic C₃-C₄ alkyl. Preferably, the alkyl group is unsubstituted alkyl group. Preferably, the alkyl group is a linear C₁-C₅, C₁-C₄, C₁-C₃, C₁-C₂ alkyl group, such as methyl group.

As used herein, the term “heteroalkyl” refers to alkyl groups where one or more carbon atoms are replaced with a heteroatom, such as, O, N, or S. Heteroalkyl group can be linear, branched, or cyclic. A heteroalkyl can be a linear C₁-C₃₀ heteroalkyl, a branched C₃-C₃₀ heteroalkyl, a cyclic C₂-C₃₀ heteroalkyl, a linear C₁-C₃₀ heteroalkyl or a branched C₃-C₃₀ heteroalkyl, a linear C₁-C₃₀ heteroalkyl or a cyclic C₂-C₃₀ heteroalkyl, a branched C₃-C₃₀ heteroalkyl or a cyclic C₂-C₃₀ heteroalkyl. Optionally, heteroalkyl groups have up to 20 carbon atoms. A heteroalkyl can be a linear C₁-C₂₀ heteroalkyl, a branched C₃-C₂₀ heteroalkyl, a cyclic C₂-C₂₀ heteroalkyl, a linear C₁-C₂₀ heteroalkyl or a branched C₃-C₂₀ heteroalkyl, a branched C₃-C₂₀ heteroalkyl or a cyclic C₂-C₂₀ heteroalkyl, or a linear C₁-C₂₀ heteroalkyl or a cyclic C₂-C₂₀ heteroalkyl. Optionally, heteroalkyl groups have up to 10 carbon atoms. A heteroalkyl can be a linear C₁-C₁₀ heteroalkyl, a branched C₃-C₁₀ heteroalkyl, a cyclic C₂-C₁₀ heteroalkyl, a linear C₁-C₁₀ heteroalkyl or a branched C₃-C₁₀ heteroalkyl, a branched C₃-C₁₀ heteroalkyl or a cyclic C₂-C₁₀ heteroalkyl, or a linear C₁-C₁₀ heteroalkyl or a cyclic C₂-C₁₀ heteroalkyl. Optionally, heteroalkyl groups have up to 6 carbon atoms. A heteroalkyl can be a linear C₁-C₆ heteroalkyl, a branched C₃-C₆ heteroalkyl, a cyclic C₂-C₆ heteroalkyl, a linear C₁-C₆ heteroalkyl or a branched C₃-C₆ heteroalkyl, a branched C₃-C₆ heteroalkyl or a cyclic C₂-C₆ heteroalkyl, or a linear C₁-C₆ heteroalkyl or a cyclic C₂-C₆ heteroalkyl. Optionally, heteroalkyl groups have up to four carbons. A heteroalkyl can be a linear C₁-C₄ heteroalkyl, a branched C₃-C₄ heteroalkyl, a cyclic C₂-C₄ heteroalkyl, a linear C₁-C₄ heteroalkyl or a branched C₃-C₄ heteroalkyl, a branched C₃-C₄ heteroalkyl or a cyclic C₂-C₄ heteroalkyl, or a linear C₁-C₄ heteroalkyl or a cyclic C₂-C₄ heteroalkyl.

As used herein, the term “alkenyl” refers to univalent groups derived from alkenes by removal of a hydrogen atom from any carbon atom. Alkenes are unsaturated hydrocarbons that contain at least one carbon-carbon double bond. Alkenyl group can be linear, branched, or cyclic. An alkenyl can be a linear C₂-C₃₀ alkenyl, a branched C₄-C₃₀ alkenyl, a cyclic C₃-C₃₀ alkenyl, a linear C₂-C₃₀ alkenyl or a branched C₄-C₃₀ alkenyl, a linear C₂-C₃₀ alkenyl or a cyclic C₃-C₃₀ alkenyl, a branched C₄-C₃₀ alkenyl or a cyclic C₃-C₃₀ alkenyl. Optionally, alkenyl groups have up to 20 carbon atoms. An alkenyl can be a linear C₂-C₂₀ alkenyl, a branched C₄-C₂₀ alkenyl, a cyclic C₃-C₂₀ alkenyl, a linear C₂-C₂₀ alkenyl or a branched C₄-C₂₀ alkenyl, a linear C₂-C₂₀ alkenyl or a cyclic C₃-C₂₀ alkenyl, a branched C₄-C₂₀ alkenyl or a cyclic C₃-C₂₀ alkenyl. Optionally, alkenyl groups have two to 10 carbon atoms. An alkenyl can be a linear C₂-C₁₀ alkenyl, a branched C₄-C₁₀ alkenyl, a cyclic C₃-C₁₀ alkenyl, a linear C₂-C₁₀ alkenyl or a branched C₄-C₁₀ alkenyl, a linear C₂-C₁₀ alkenyl or a cyclic C₃-C₁₀ alkenyl, a branched C₄-C₁₀ alkenyl or a cyclic C₃-C₁₀ alkenyl. Optionally, alkenyl groups have two to 6 carbon atoms. An alkenyl can be a linear C₂-C₆ alkenyl, a branched C₄-C₆ alkenyl, a cyclic C₃-C₆ alkenyl, a linear C₂-C₆ alkenyl or a branched C₄-C₆ alkenyl, a linear C₂-C₆ alkenyl or a cyclic C₃-C₆ alkenyl, a branched C₄-C₆ alkenyl or a cyclic C₃-C₆ alkenyl. Optionally, alkenyl groups have two to four carbons. An alkenyl can be a linear C₂-C₄ alkenyl, a cyclic C₃-C₄ alkenyl, a linear C₂-C₄ alkenyl or a cyclic C₃-C₄ alkenyl.

As used herein, the term “heteroalkenyl” refers to alkenyl groups in which one or more doubly bonded carbon atoms are replaced by a heteroatom. Heteroalkenyl group can be linear, branched, or cyclic. A heteroalkenyl can be a linear C₂-C₃₀ heteroalkenyl, a branched C₃-C₃₀ heteroalkenyl, a cyclic C₂-C₃₀ heteroalkenyl, a linear C₂-C₃₀ heteroalkenyl or a branched C₃-C₃₀ heteroalkenyl, a linear C₂-C₃₀ heteroalkenyl or a cyclic C₂-C₃₀ heteroalkenyl, a branched C₃-C₃₀ heteroalkenyl or a cyclic C₂-C₃₀ heteroalkenyl. Optionally, heteroalkenyl groups have up to 20 carbon atoms. A heteroalkenyl can be a linear C₂-C₂₀ heteroalkenyl, a branched C₃-C₂₀ heteroalkenyl, a cyclic C₂-C₂₀ heteroalkenyl, a linear C₂-C₂₀ heteroalkenyl or a branched C₃-C₂₀ heteroalkenyl, a linear C₂-C₂₀ heteroalkenyl or a cyclic C₂-C₂₀ heteroalkenyl, a branched C₃-C₂₀ heteroalkenyl or a cyclic C₂-C₂₀ heteroalkenyl. Optionally, heteroalkenyl groups have up to 10 carbon atoms. A heteroalkenyl can be a linear C₂-C₁₀ heteroalkenyl, a branched C₃-C₁₀ heteroalkenyl, a cyclic C₂-C₁₀ heteroalkenyl, a linear C₂-C₁₀ heteroalkenyl or a branched C₃-C₁₀ heteroalkenyl, a linear C₂-C₁₀ heteroalkenyl or a cyclic C₂-C₁₀ heteroalkenyl, a branched C₃-C₁₀ heteroalkenyl or a cyclic C₂-C₁₀ heteroalkenyl. Optionally, heteroalkenyl groups have two to 6 carbon atoms. A heteroalkenyl can be a linear C₂-C₆ heteroalkenyl, a branched C₃-C₆ heteroalkenyl, a cyclic C₂-C₆ heteroalkenyl, a linear C₂-C₆ heteroalkenyl or a branched C₃-C₆ heteroalkenyl, a linear C₂-C₆ heteroalkenyl or a cyclic C₂-C₆ heteroalkenyl, a branched C₃-C₆ heteroalkenyl or a cyclic C₂-C₆ heteroalkenyl. Optionally, heteroalkenyl groups have two to four carbons. A heteroalkenyl can be a linear C₂-C₄ heteroalkenyl, a branched C₃-C₄ heteroalkenyl, a cyclic C₂-C₄ heteroalkenyl, a linear C₂-C₄ heteroalkenyl or a branched C₃-C₄ heteroalkenyl, a linear C₂-C₄ heteroalkenyl or a cyclic C₂-C₄ heteroalkenyl, a branched C₃-C₄ heteroalkenyl or a cyclic C₂-C₄ heteroalkenyl.

As used herein, the term “alkynyl” refers to univalent groups derived from alkenes by removal of a hydrogen atom from any carbon atom. Alkynes are unsaturated hydrocarbons that contain at least one carbon-carbon triple bond. Alkynyl group can be linear, branched, or cyclic. An alkynyl can be a linear C₂-C₃₀ alkynyl, a branched C₄-C₃₀ alkynyl, a cyclic C₃-C₃₀ alkynyl, a linear C₂-C₃₀ alkynyl or a branched C₄-C₃₀ alkynyl, a linear C₂-C₃₀ alkynyl or a cyclic C₃-C₃₀ alkynyl, a branched C₄-C₃₀ alkynyl or a cyclic C₃-C₃₀ alkynyl. Optionally, alkynyl groups have up to 20 carbon atoms. An alkynyl can be a linear C₂-C₂₀ alkynyl, a branched C₄-C₂₀ alkynyl, a cyclic C₃-C₂₀ alkynyl, a linear C₂-C₂₀ alkynyl or a branched C₄-C₂₀ alkynyl, a branched C₄-C₂₀ alkynyl or a cyclic C₃-C₂₀ alkynyl. Optionally, alkynyl groups have up to 10 carbon atoms. An alkynyl can be a linear C₂-C₁₀ alkynyl, a branched C₄-C₁₀ alkynyl, a cyclic C₃-C₁₀ alkynyl, a linear C₂-C₂₀ alkynyl or a branched C₄-C₁₀ alkynyl, a branched C₄-C₂₀ alkynyl or a cyclic C₃-C₁₀ alkynyl, a linear C₂-C₂₀ alkynyl or a cyclic C₃-C₂₀ alkynyl. Optionally, alkynyl groups have up to 6 carbon atoms. An alkynyl can be a linear C₂-C₆ alkynyl, a branched C₄-C₆ alkynyl, a cyclic C₃-C₆ alkynyl, a linear C₂-C₆ alkynyl or a branched C₄-C₆ alkynyl, a branched C₄-C₆ alkynyl or a cyclic C₃-C₆ alkynyl, a linear C₂-C₆ alkynyl or a cyclic C₃-C₆ alkynyl. Optionally, alkynyl groups have up to four carbons. An alkynyl can be a linear C₂-C₄ alkynyl, a cyclic C₃-C₄ alkynyl, a linear C₂-C₄ alkynyl or a cyclic C₃-C₄ alkynyl.

As used herein, the term “heteroalkynyl” refers to alkynyl groups in which one or more triply bonded carbon atoms are replaced by a heteroatom. Heteroalkynyl group can be linear, branched, or cyclic. A heteroalkynyl can be a linear C₂-C₃₀ heteroalkynyl, a branched C₃-C₃₀ heteroalkynyl, a cyclic C₂-C₃₀ heteroalkynyl, a linear C₂-C₃₀ heteroalkynyl or a branched C₃-C₃₀ heteroalkynyl, a linear C₂-C₃₀ heteroalkynyl or a cyclic C₂-C₃₀ heteroalkynyl, a branched C₃-C₃₀ heteroalkynyl or a cyclic C₂-C₃₀ heteroalkynyl. Optionally, heteroalkynyl groups have up to 20 carbon atoms. A heteroalkynyl can be a linear C₂-C₂₀ heteroalkynyl, a branched C₃-C₂₀ heteroalkynyl, a cyclic C₂-C₂₀ heteroalkynyl, a linear C₂-C₂₀ heteroalkynyl or a branched C₃-C₂₀ heteroalkynyl, a branched C₃-C₂₀ heteroalkynyl or a cyclic C₂-C₂₀ heteroalkynyl, a linear C₂-C₂₀ heteroalkynyl or a cyclic C₂-C₂₀ heteroalkynyl. Optionally, heteroalkynyl groups have up to 10 carbon atoms. A heteroalkynyl can be a linear C₂-C₁₀ heteroalkynyl, a branched C₃-C₁₀ heteroalkynyl, a cyclic C₂-C₁₀ heteroalkynyl, a linear C₂-C₁₀ heteroalkynyl or a branched C₃-C₁₀ heteroalkynyl, a branched C₃-C₁₀ heteroalkynyl or a cyclic C₂-C₁₀ heteroalkynyl, a linear C₂-C₁₀ heteroalkynyl or a cyclic C₂-C₁₀ heteroalkynyl. Optionally, heteroalkynyl groups have two to 6 carbon atoms. A heteroalkynyl can be a linear C₂-C₆ heteroalkynyl, a branched C₃-C₆ heteroalkynyl, a cyclic C₂-C₆ heteroalkynyl, a linear C₂-C₆ heteroalkynyl or a branched C₃-C₆ heteroalkynyl, a branched C₃-C₆ heteroalkynyl or a cyclic C₂-C₆ heteroalkynyl, a linear C₂-C₆ heteroalkynyl or a cyclic C₂-C₆ heteroalkynyl. Optionally, heteroalkynyl groups have up to four carbons. A heteroalkynyl can be a linear C₂-C₄ heteroalkynyl, a branched C₃-C₄ heteroalkynyl, a cyclic C₂-C₄ heteroalkynyl, a linear C₂-C₄ heteroalkynyl or a branched C₃-C₄ heteroalkynyl, a branched C₃-C₄ heteroalkynyl or a cyclic C₂-C₄ heteroalkynyl, a linear C₂-C₄ heteroalkynyl or a cyclic C₂-C₄ heteroalkynyl.

As used herein, the term “aryl” refers to univalent groups derived from arenes by removal of a hydrogen atom from a ring atom. Arenes are monocyclic and polycyclic aromatic hydrocarbons. In polycyclic aryl groups, the rings can be attached together in a pendant manner or can be fused. Aaryl group can have six to 50 carbon atoms. An aryl can be a branched C₆-C₅₀ aryl, a monocyclic C₆-C₅₀ aryl, a polycyclic C₆-C₅₀ aryl, a branched polycyclic C₆-C₅₀ aryl, a fused poly cyclic C₆-C₅₀ aryl, or a branched fused polycyclic C₆-C₅₀ aryl. Optionally, aryl groups have six to 30 carbon atoms, i.e., C₆-C₃₀ aryl. A C₆-C₃₀ aryl can be a branched C₆-C₃₀ aryl, a monocyclic C₆-C₃₀ aryl, a polycyclic C₆-C₃₀ aryl, a branched polycyclic C₆-C₃₀ aryl, a fused polycyclic C₆-C₃₀ aryl, or a branched fused polycyclic C₆-C₃₀ aryl. Optionally, aryl groups have six to 20 carbon atoms, i.e., C₆-C₂₀ aryl. A C₆-C₂₀ aryl can be a branched C₆-C₂₀ aryl, a monocyclic C₆-C₂₀ aryl, a polycyclic C₆-C₂₀ aryl, a branched polycyclic C₆-C₂₀ aryl, a fused polycyclic C₆-C₂₀ aryl, or a branched fused polycyclic C₆-C₂₀ aryl. Optionally, aryl groups have six to twelve carbon atoms, i.e., C₆-C₁₂ aryl. A C₆-C₁₂ aryl can be a branched C₆-C₁₂ aryl, a monocyclic C₆-C₁₂ aryl, a polycyclic C₆-C₁₂ aryl, a branched polycyclic C₆-C₁₂ aryl, a fused polycyclic C₆-C₁₂ aryl, or a branched fused polycyclic C₆-C₁₂ aryl. Optionally, C₆-C₁₂ aryl groups have six to eleven carbon atoms, i.e., C₆-C₁₁ aryl. A C₆-C₁₁ aryl can be a branched C₆-C₁₁ aryl, a monocyclic C₆-C₁₁ aryl, a polycyclic C₆-C₁₁ aryl, a branched polycyclic C₆-C₁₁ aryl, a fused polycyclic C₆-C₁₁ aryl, or a branched fused polycyclic C₆-C₁₁ aryl. Optionally, C₆-C₁₂ aryl groups have six to nine carbon atoms, i.e., C₆-C₉ aryl. A C₆-C₉ aryl can be a branched C₆-C₉ aryl, a monocyclic C₆-C₉ aryl, a polycyclic C₆-C₉ aryl, a branched polycyclic C₆-C₉ aryl, a fused polycyclic C₆-C₉ aryl, or a branched fused polycyclic C₆-C₉ aryl. Optionally, C₆-C₁₂ aryl groups have six carbon atoms, i.e., C₆ aryl. A C₆ aryl can be a branched C₆ aryl or a monocyclic C₆ aryl.

As used herein, the term “heteroaryl” refers to univalent groups derived from heteroarenes by removal of a hydrogen atom from a ring atom. Heteroarenes are heterocyclic compounds derived from arenes by replacement of one or more methine (—C═) and/or vinylene (—CH═CH—) groups by trivalent or divalent heteroatoms, respectively, in such a way as to maintain the continuous R-electron system characteristic of aromatic systems and a number of out-of-plane R-electrons corresponding to the Hickel rule (4n+2). In polycyclic heteroaryl groups, the rings can be attached together in a pendant manner or can be fused. Heteroaryl group can have three to 50 carbon atoms, i.e., C₃-C₅₀ heteroaryl. A C₃-C₅₀ heteroaryl can be a branched C₃-C₅₀ heteroaryl, a monocyclic C₃-C₅₀ heteroaryl, a polycyclic C₃-C₅₀ heteroaryl, a branched polycyclic C₃-C₅₀ heteroaryl, a fused polycyclic C₃-C₅₀ heteroaryl, or a branched fused polycyclic C₃-C₅₀ heteroaryl. Optionally, heteroaryl groups have six to 30 carbon atoms, i.e., C₆-C₃₀ heteroaryl. A C₆-C₃₀ heteroaryl can be a branched C₆-C₃₀ heteroaryl, a monocyclic C₆-C₃₀ heteroaryl, a polycyclic C₆-C₃₀ heteroaryl, a branched polycyclic C₆-C₃₀ heteroaryl, a fused polycyclic C₆-C₃₀ heteroaryl, or a branched fused polycyclic C₆-C₃₀ heteroaryl. Optionally, heteroaryl groups have six to 20 carbon atoms, i.e., C₆-C₂₀ heteroaryl. A C₆-C₂₀ heteroaryl can be a branched C₆-C₂₀ heteroaryl, a monocyclic C₆-C₂₀ heteroaryl, a polycyclic C₆-C₂₀ heteroaryl, a branched polycyclic C₆-C₂₀ heteroaryl, a fused polycyclic C₆-C₂₀ heteroaryl, or a branched fused polycyclic C₆-C₂0 heteroaryl. Optionally, heteroaryl groups have six to twelve carbon atoms, i.e., C₆-C₁₂ heteroaryl. A C₆-C₁₂ heteroaryl can be a branched C₆-C₁₂ heteroaryl, a monocyclic C₆-C₁₂ heteroaryl, a polycyclic C₆-C₁₂ heteroaryl, a branched polycyclic C₆-C₁₂ heteroaryl, a fused polycyclic C₆-C₁₂ heteroaryl, or a branched fused polycyclic C₆-C₁₂ heteroaryl. Optionally, C₆-C₁₂ heteroaryl groups have six to eleven carbon atoms, i.e., C₆-C₁₁ heteroaryl. A C₆-C₁₁ heteroaryl can be a branched C₆-C₁₁ heteroaryl, a monocyclic C₆-C₁₁ heteroaryl, a polycyclic C₆-C₁₁ heteroaryl, a branched polycyclic C₆-C₁₁ heteroaryl, a fused polycyclic C₆-C₁₁ heteroaryl, or a branched fused polycyclic C₆-C₁₁ heteroaryl. Optionally, C₆-C₁₂ heteroaryl groups have six to nine carbon atoms, i.e., C₆-C₉ heteroaryl. A C₆-C₉ heteroaryl can be a branched C₆-C₉ heteroaryl, a monocyclic C₆-C₉ heteroaryl, a polycyclic C₆-C₉ heteroaryl, a branched polycyclic C₆-C₉ heteroaryl, a fused polycyclic C₆-C₉ heteroaryl, or a branched fused polycyclic C₆-C₉ heteroaryl. Optionally, C₆-C₁₂ heteroaryl groups have six carbon atoms, i.e., C₆ heteroaryl. A C₆ heteroaryl can be a branched C₆ heteroaryl, a monocyclic C₆ heteroaryl, a polycyclic C₆ heteroaryl, a branched polycyclic C₆ heteroaryl, a fused polycyclic C₆ heteroaryl, or a branched fused polycyclic C₆ heteroaryl.

As used herein, the term “substituted,” means that the chemical group or moiety contains one or more substituents replacing the hydrogen atoms in the chemical group or moiety. The substituents include, but are not limited to:
a halogen atom, an alkyl group, a cycloalkyl group, a heteroalkyl group, a cycloheteroalkyl group, an alkenyl group, a heteroalkenyl group, an alkynyl group, a heteroalkynyl group, an aryl group, a heteroaryl group, a polyaryl group, a polyheteroaryl group, —OH, —SH, —NH₂, —N₃, —OCN, —NCO, —ONO₂, —CN, —NC, —ONO, —CONH₂, —NO, —NO₂, —ONH₂, —SCN, —SNCS, —CF₃, —CH₂CF₃, —CH₂C₁, —CHCl₂, —CH₂NH₂, —NHCOH, —C HO, —COCl, —COF, —COBr, —COOH, —SO₃H, —CH₂SO₂CH₃, —PO₃H₂, —OPO₃H₂, —P(═O)(OR^{T1′})(OR^{T2′}), —OP(═O)(OR^{T1′})(OR^{T2′}), —BR^{T1′}(OR^{T2′}), —B(OR^{T1′})(OR^{T2′}), or -G′R^{T1′} in which -T′ is —O—, —S—, —NR^{T2′}—, —C(═O)—, —S(═O)—, —SO₂—, —C(═O)O—, —C(═O)NR^{T2′}—, —OC(═O)—, —NR^{T2′}C(═O)—, —OC(═O)O—, —OC(═O)NR^{T2′}—, —NR^{T2′}C(═O)O—, —NR^{T2′}C(═O)NR^{T3′}—, —C(═S)—, —C(═S)S—, —SC(═S)—, —SC(═S)S—, —C(═NR^{T2′})—, —C(═NR^{T2′})O—, —C(═NR^{T2′})NR^{T3′}—, —OC(═NR^{T2′})—, —NR^{T2′}C(═NR^{T3′})—, —NR^{T2′}SO₂—, —C(═NR^{T2′})NR^{T3′}—, —OC(═NR^{T2′})—, —NR^{T2′}C(═NR^{T3′})—, —NR^{T2′}SO₂—, —NR^{T2′}SO₂NR^{T3′}—, —NR^{T2′}C(═S)—, —SC(═S)NR^{T2′}—, —NR^{T2′}C(═S)S—, —NR^{T2′}C(═S)NR^{T3′}—, —SC(═NR^{T2′})—, —C(═S)NR^{T2′}—, —OC(═S)NR^{T2′}—, —NR^{T2′}C(═S)O—, —SC(═O)NR^{T2′}—, —NR^{T2′}C(═O)S—, —C(═O)S—, —SC(═O)—, —SC(═O)S—, —C(═S)O—, —OC(═S)—, —OC(═S)O—, —SO₂NR^{T2′}—, —BR^{T2′}—, or —PR^{T2′}—; where each occurrence of R^{T1′}, R^{T2′}, and R^{T3′} is, independently, a hydrogen atom, a halogen atom, an alkyl group, a heteroalkyl group, an alkenyl group, a heteroalkenyl group, an alkynyl group, a heteroalkynyl group, an aryl group, or a heteroaryl group.

In some instances, “substituted” also refers to one or more substitutions of one or more of the carbon atoms in a carbon chain (e.g., alkyl, alkenyl, alkynyl, and aryl groups) by a heteroatom, such as, but not limited to, nitrogen, oxygen, and sulfur.

It is understood that “substitution” or “substituted” includes the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc.

Use of the term “about” is intended to describe values either above or below the stated value in a range of approx. +/−10%; in other embodiments the values may range in value either above or below the stated value in a range of approx. +/−5%. The preceding ranges are intended to be made clear by context, and no further limitation is implied.

Numerical ranges disclosed in the present application of any type, disclose individually each possible number that such a range could reasonably encompass, as well as any sub-ranges and combinations of sub-ranges encompassed therein.

### II. Compositions

Bicyclic heterocyclic compounds and their derivatives (together also referred to therein as “compounds”) having antiviral properties and low toxicity have been developed. These have broad antiviral properties and should be suitable for use in the prophylaxis and/or treatment of multiple classes of viruses.

Generally, the compounds mimic RNA and/or DNA bases through base pairing with RNA and/or DNA nucleosides, becoming incorporated into the viral RNA and/or DNA strand, resulting in viral mutagenesis (Strombaugh et al., Nucleic Acids Research, 37(7):2294-2312 (2009); Jin et al., POLS one, 8(7):e68347 (2013)). Compounds that are structurally comparable to RNA and/or DNA nucleosides (also referred to herein as “nucleoside analogue prodrugs”) act as competitive enzymatic inhibitors when incorporated into newly synthesized viral RNA and/or DNA. It is known that nucleoside analogue prodrugs can be converted to active metabolite drugs, 5′ ribose triphosphate (RTP) (see, e.g., Good, et al., Antimicrobial Agents and Chemotherapy, 65(4):e02479-20, 2021). The nucleoside analogue prodrugs can accelerate the absorption of these prodrugs into cells and the formation of the active metabolite drug, 5′ ribose triphosphate (RTP), which base-pairs with RNA and/or DNA bases and gets incorporated into the viral RNA and/or DNA strand, resulting in viral mutagenesis (see, for example, U.S. Pat. No. 7,018,989 to McGuigan et al.). The overall structure of these compounds for base pairing may render them useful in treating medical conditions in addition to viruses.

Typically, the compounds can form at least two hydrogen bonds with RNA nucleosides and hence show high potential to mimic RNA and/or DNA bases. Additionally, the compounds eliminate the free rotation of the amide group of the currently approved antivirals (e.g., Ribavirin and Favipiravir), thereby lowering the toxicity (e.g. teratogenicity and embryo toxicity) of these compounds. In one embodiment, the compounds possess enhanced antiviral activity and reduced toxicity (e.g. teratogenicity and embryo toxicity) compared with the currently approved antivirals, such as Ribavirin and Favipiravir.

Pharmaceutical compositions and formulations containing the compounds are also disclosed.

A. Compounds

1. Bicyclic Heterocyclic Compounds and Derivatives Thereof

The compounds can have the structures of Formula I.

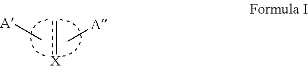

where A′ and A″ are independently an unsubstituted heterocyclic group or a substituted heterocyclic group; and
where X′ is a carbon or a nitrogen,
where the substituents are independently a phosphoramidate, a heterocyclic group, a halogen, a sulfonic acid, an azide group, a cyanate group, an isocyanate group, a nitrate group, a nitrile group, an isonitrile group, a nitrosooxy group, a nitroso group, a nitro group, an aldehyde group, an alkoxy group, an acyl halide group, a carboxylic acid group, a carboxylate group, an alkyl group, a heteroalkyl group, an alkenyl group, a heteroalkenyl group, an alkynyl group, a heteroalkynyl group, an aryl group, a heteroaryl group, polyaryl group, a heteropoly aryl group, a alkyl aryl group, an amino group, a hydroxyl group, a thiol group, a sulfonyl group, an ester group, an aliphatic ester group, an amide group, an aliphatic amide group, an azo group, an acyl group, a carbonyl group, a carbonate ester group, an ether group, an oxo, an aminooxy group, or a hydroxyamino group.

In some embodiments, A′ and A″ are independently an unsubstituted heterocyclic group. In some embodiments, A′ and A″ are independently a substituted heterocyclic group.

In some embodiments, A′ and A″ are independently an unsubstituted or substituted pyrrolyl group, an unsubstituted or substituted imidazolyl group, an unsubstituted or substituted pyrazolyl group, an unsubstituted or substituted triazolyl group, an unsubstituted or substituted thiazolyl group, an unsubstituted or substituted isothiazolyl group, an unsubstituted or substituted oxazolyl group, an unsubstituted or substituted oxazinyl group, an unsubstituted or substituted pyrrolidinyl group, an unsubstituted or substituted pyrrolinyl group, an unsubstituted or substituted imidazolidinyl group, an unsubstituted or substituted imidazolinyl group, an unsubstituted or substituted pyrazolidinyl group, an unsubstituted or substituted tetrahydrofuranyl group, an unsubstituted or substituted pyranyl group, an unsubstituted or substituted pyronyl group, an unsubstituted or substituted pyridyl group, an unsubstituted or substituted pyrazinyl group, an unsubstituted or substituted pyridazinyl group, an unsubstituted or substituted piperidyl group, an unsubstituted or substituted piperazinyl group, an unsubstituted or substituted morpholinyl group, an unsubstituted or substituted benzofuranyl group, an unsubstituted or substituted isobenzofuryl group, an unsubstituted or substituted indolyl group, an unsubstituted or substituted oxyindolyl group, an unsubstituted or substituted isoindolyl group, an unsubstituted or substituted indazolyl group, an unsubstituted or substituted indolinyl group, an unsubstituted or substituted 7-azaindolyl group, an unsubstituted or substituted isoindazolyl group, an unsubstituted or substituted benzopyranyl group, an unsubstituted or substituted coumarinyl group, an unsubstituted or substituted isocoumarinyl group, an unsubstituted or substituted quinolyl group, an unsubstituted or substituted iso-quinolyl group, an unsubstituted or substituted naphthridinyl group, an unsubstituted or substituted cinnolinyl group, an unsubstituted or substituted quinazolinyl group, an unsubstituted or substituted pyridopyridyl group, an unsubstituted or substituted benzoxazinyl group, an unsubstituted or substituted quinoxadinyl group, an unsubstituted or substituted chromenyl group, an unsubstituted or substituted chromanyl group, an unsubstituted or substituted isochrcmanyl group, or an unsubstituted or substituted carbolinyl group.

In some embodiments, A′ and A″ are independently an unsubstituted or substituted pyrazinyl group, an unsubstituted or substituted oxazinyl group, an unsubstituted or substituted triazinyl group, an unsubstituted or substituted triazolyl group, an unsubstituted or substituted pyrazolyl group, an unsubstituted or substituted pyrrolyl group, an unsubstituted or substituted pyridazinyl group, an unsubstituted or substituted pyrimidinyl group, an unsubstituted or substituted pyridinyl group, or an unsubstituted or substituted imidazolyl group. In some embodiments, A′ and A″ are independently a substituted pyrazinyl group, a substituted oxazinyl group, a substituted triazinyl group, a substituted triazolyl group, a substituted pyrazolyl group, a substituted pyrrolyl group, a substituted pyridazinyl group, a substituted pyrimidinyl group, a substituted pyridinyl group, or a substituted imidazolyl group.

In some embodiments, the compounds have the structures of Formula IIa or Formula IIIa.

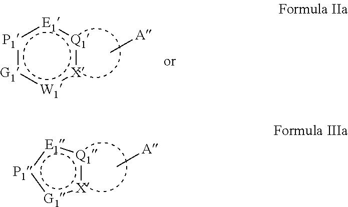

(a) where A″ and X′ are as defined above; (b) where E₁′, E₁″, P₁′, P₁″, G₁′, G₁″, and W₁′ are independently CR₂₀ or NR₂₁, and R₂₀ and R₂₁ are independently absent, a hydrogen, a halogen, a nitrile group, a nitro group, an oxo group, an ester group, an unsubstituted aliphatic ester group, a substituted aliphatic ester group, an ether group, an unsubstituted aliphatic ether group, a substituted aliphatic ether group, an unsubstituted amino group, a substituted amino group, an unsubstituted aliphatic amino group, a substituted aliphatic amino group, an amide group, an unsubstituted aliphatic-amide group, a substituted aliphatic-amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted heterocyclic group, a substituted heterocyclic group, an unsubstituted heterocyclic-aliphatic group, or a substituted heterocyclic-aliphatic group; (c) where Q₁′ and Q₁″ are independently CR₂₀ and R₂₀ can be as defined above, for example, R₂₀ is absent or hydrogen according to valency; and (d) where the substituents are independently a phosphoramidate, a heterocyclic group, a halogen, a sulfonic acid, an azide group, a cyanate group, an isocyanate group, a nitrate group, a nitrile group, an isonitrile group, a nitrosooxy group, a nitroso group, a nitro group, an aldehyde group, an alkoxy group, an acyl halide group, a carboxylic acid group, a carboxylate group, an alkyl group, a heteroalkyl group, an alkenyl group, a heteroalkenyl group, an alkynyl group, a heteroalkynyl group, an aryl group, a heteroaryl group, polyaryl group, a heteropoly aryl group, a alkyl aryl group, an amino group, an ester group, a hydroxyl group, a thiol group, a sulfonyl group, an ester group, an aliphatic ester group, an amide group, an aliphatic amide group, an azo group, an acyl group, a carbonyl group, a carbonate ester group, an ether group, an aminooxy group, or a hydroxyamino group.

In some embodiments, the compounds have the structures of Formula II′a.

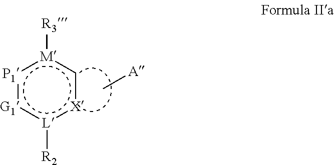

(a) where A″, X′, P₁′, and G₁′ are as defined above; (b) where L′ and M″ are independently a carbon or a nitrogen; (c) where R₂ is absent, a hydrogen, an unsubstituted aliphatic group, a substituted aliphatic group, an unsubstituted aryl group, a substituted aryl group, an unsubstituted aryl-aliphatic group, a substituted aryl-aliphatic group, an unsubstituted aliphatic ether group, or a substituted aliphatic ether group; (d) R₃′″ is absent, a hydrogen, an unsubstituted aliphatic group, a substituted aliphatic group, an unsubstituted amide group, a substituted amide group, an unsubstituted aliphatic ester group, a substituted aliphatic ester group, an unsubstituted aliphatic ether group, a substituted aliphatic ether group, an unsubstituted heterocyclic group, a substituted heterocyclic group, an unsubstituted heterocyclic-aliphatic group, or a substituted heterocyclic-aliphatic group; and (e) where the substituents are as defined above.

In some embodiments, the compounds have the structures of Formula IIb, II′b, or Formula IIIb.

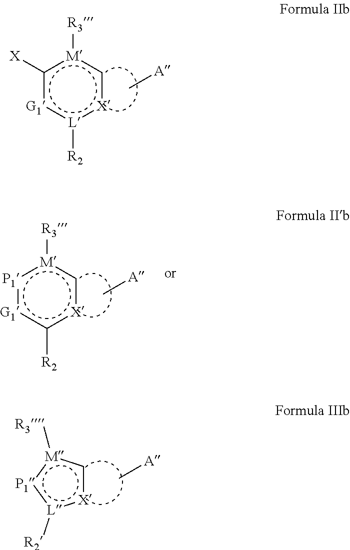

(a) where A″ and X′ are as defined above; (b) where L′, M′, L″, and M″ are independently a carbon or a nitrogen; (c) where P₁′, P₁″ and G₁′ are independently CR₂₀ or NR₂₁, and R₂₀ and R₂₁ are independently absent, a hydrogen, a halogen, a nitrile group, a nitro group, an oxo group, an ester group, an unsubstituted aliphatic ester group, a substituted aliphatic ester group, an ether group, an unsubstituted aliphatic ether group, a substituted aliphatic ether group, an unsubstituted amino group, a substituted amino group, an unsubstituted aliphatic amino group, a substituted aliphatic amino group, an amide group, an aliphatic-amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted heterocyclic group, a substituted heterocyclic group, an unsubstituted heterocyclic-aliphatic group, or a substituted heterocyclic-aliphatic group; (d) where X is a hydrogen, a halogen, a nitrile group, a nitro group, an ester group, an unsubstituted aliphatic ester group, a substituted aliphatic ester group, an unsubstituted amino group, a substituted amino group, an amide group, an unsubstituted aliphatic-amide group, a substituted aliphatic-amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted heterocyclic group, a substituted heterocyclic group, an unsubstituted heterocyclic-aliphatic group, or a substituted heterocyclic-aliphatic group; (e) where R₂ and R₂′ are independently absent, a hydrogen, an unsubstituted aliphatic group, a substituted aliphatic group, an unsubstituted aryl group, a substituted aryl group, an unsubstituted aryl-aliphatic group, a substituted aryl-aliphatic group, an unsubstituted aliphatic ether group, or a substituted aliphatic ether group; (f) where R₃′″ and R₃″″ are independently absent, a hydrogen, an unsubstituted aliphatic group, a substituted aliphatic group, an unsubstituted amide group, a substituted amide group, an unsubstituted aliphatic ester group, a substituted aliphatic ester group, an unsubstituted aliphatic ether group, a substituted aliphatic ether group, an unsubstituted heterocyclic group, a substituted heterocyclic group, an unsubstituted heterocyclic-aliphatic group, or a substituted heterocyclic-aliphatic group; and (g) where the substituents are as defined above.

In some embodiments, the compounds have the structures of Formula II, Formula III, Formula II′, Formula III′, Formula II″, or Formula II′″.

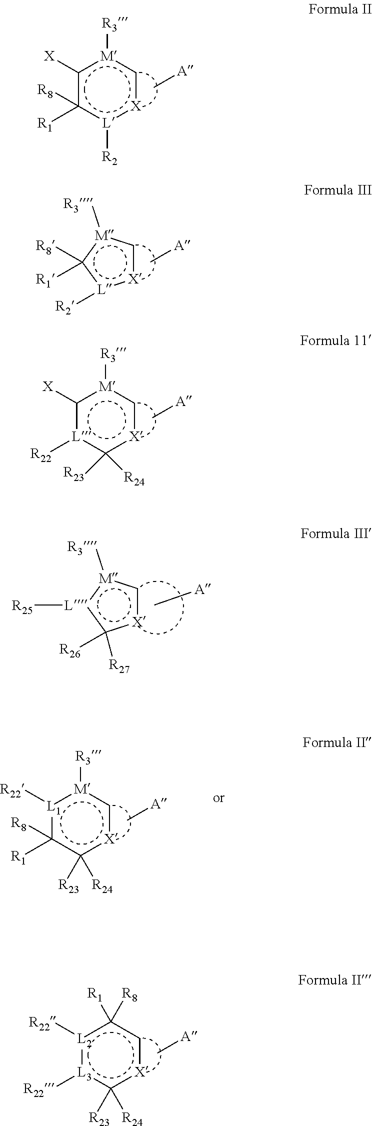

where X′ and A″ are as defined above;
where L′, M′, L″, M″, L′″, L″″, and L₁-L₃ are independently a carbon or a nitrogen;
where X is a hydrogen, a halogen, a nitrile group, a nitro group, an ester group, an unsubstituted aliphatic ester group, a substituted aliphatic ester group, an unsubstituted amino group, a substituted amino group, an amide group, an unsubstituted aliphatic-amide group, a substituted aliphatic-amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted heterocyclic group, a substituted heterocyclic group, an unsubstituted heterocyclic-aliphatic group, or a substituted heterocyclic-aliphatic group;
where R₁, R₁′, R₂₃, and R₂₆ are independently a hydrogen, a halogen, a nitrile group, a nitro group, an ester group, an unsubstituted aliphatic ester group, a substituted aliphatic ester group, an unsubstituted amino group, a substituted amino group, an unsubstituted aliphatic amino group, a substituted aliphatic amino group, an amide group, an unsubstituted aliphatic amide group, a substituted aliphatic amide group, an azide group, an unsubstituted aliphatic group, a substituted aliphatic group, an unsubstituted aryl group, a substituted aryl group, an unsubstituted aryl-aliphatic group, a substituted aryl-aliphatic group, an ether group, an unsubstituted aliphatic ether group, a substituted aliphatic ether group;
where R₂, R₂′, R₂₂, R₂₂′, R₂₂″, R₂₂′″, and R₂₅ are independently absent, a hydrogen, an unsubstituted aliphatic group, a substituted aliphatic group, an unsubstituted aryl group, a substituted aryl group, an unsubstituted aryl-aliphatic group, a substituted aryl-aliphatic group, an unsubstituted aliphatic ether group, or a substituted aliphatic ether group;
where R₃′″ and R₃″″ are independently absent, a hydrogen, an unsubstituted aliphatic group, a substituted aliphatic group, an unsubstituted amide group, a substituted amide group, an unsubstituted aliphatic ester group, a substituted aliphatic ester group, an unsubstituted aliphatic ether group, a substituted aliphatic ether group, an unsubstituted heterocyclic group, a substituted heterocyclic group, an unsubstituted heterocyclic-aliphatic group, or a substituted heterocyclic-aliphatic group;
where R₈, R₈′, R₂₄, and R₂₇ are independently absent, a hydrogen, a halogen, a nitrile group, an ester group, an unsubstituted aliphatic ester group, a substituted aliphatic ester group, an unsubstituted amino group, a substituted amino group, an unsubstituted aliphatic amino group, a substituted aliphatic amino group, an amide group, a substituted aliphatic-amide group, an unsubstituted aliphatic-amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted aliphatic ether group, a substituted aliphatic ether group, un unsubstituted heterocyclic group, a substituted heterocyclic group, an unsubstituted heterocyclic-aliphatic group, or a substituted heterocyclic-aliphatic group; and
where the substituents are as defined above.

In some embodiments, A″ of Formulae I, IIa, IIb, II, II′, II′a, II′b, II″, II′″, IIIa, IIIb, III, and/or III′ is

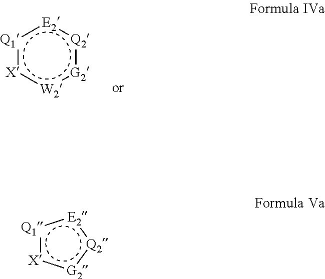

(a) where X′ is as defined above; (b) where E₂′, E₂″, G₂′, G₂″, Q₂′, Q₂″, and W₂′ are independently an oxygen, a sulfur, CR₂₀, or NR₂₁, and R₂₀ and R₂₁ are independently absent, a hydrogen, a halogen, a nitrile group, a nitro group, an oxo group, an ester group, an unsubstituted aliphatic ester group, a substituted aliphatic ester group, an ether group, an unsubstituted aliphatic ether group, a substituted aliphatic ether group, an unsubstituted amino group, a substituted amino group, an unsubstituted aliphatic amino group, a substituted aliphatic amino group, an amide group, an unsubstituted aliphatic-amide group, a substituted aliphatic-amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted heterocyclic group, a substituted heterocyclic group, an unsubstituted heterocyclic-aliphatic group, or a substituted heterocyclic-aliphatic group; (c) where Q₁′ and Q₁″ are independently CR₂₀ and R₂₀ can be as defined above, for example, R₂₀ is absent; and (d) where the substituents are as defined above.

In some embodiments, A″ of Formulae I, IIa, IIb, II, II′, II′a, II′b, II″, II′″, IIIa, IIIb, III, and/or III′ is

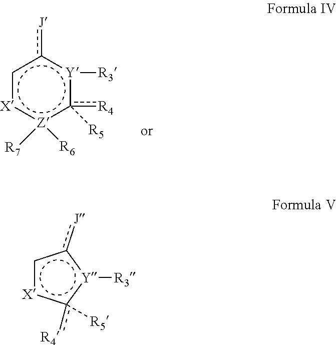

where X′ is as defined above; where Y′, Y″, and Z′ are independently a carbon, an oxygen, a nitrogen, or a sulfur;
where J′ and J″ are independently an oxygen, OR₃, a sulfur, a thiol group, an unsubstituted amino group, or a substituted amino group (e.g. a hydroxyl substituted amino group, such as

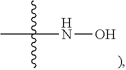

where R₃, R₃′, and R₃″ are independently absent, a hydrogen, an unsubstituted aliphatic group, a substituted aliphatic group, an unsubstituted aliphatic ester group, a substituted aliphatic ester group, an unsubstituted aliphatic ether group, a substituted aliphatic ether group, an unsubstituted heterocyclic group, a substituted heterocyclic group, an unsubstituted heterocyclic-aliphatic group, or a substituted heterocyclic-aliphatic group; and
where R₄, R₅, R₄′, R₅′, R₆, and R₇, according to valency, are independently absent, a hydrogen, an oxygen, a halogen, a nitrile group, an ester group, an unsubstituted aliphatic ester group, a substituted aliphatic ester group, an unsubstituted amino group, a substituted amino group, an unsubstituted aliphatic-amino group, a substituted aliphatic-amino group, an amide group, an aliphatic-amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted aliphatic ether group, a substituted aliphatic ether group, un unsubstituted heterocyclic group, a substituted heterocyclic group, an unsubstituted heterocyclic-aliphatic group, or a substituted heterocyclic-aliphatic group, where the substituents are as defined above.

In some embodiments of Formula IV or Formula V, Y′ and Y″ are nitrogen and Z′ is a carbon, an oxygen, a nitrogen, or a sulfur. In some embodiments of Formula IV or Formula V, Y′ and Y″ are nitrogen and Z′ is a carbon, an oxygen, or a nitrogen. In some embodiments of Formula IV or Formula V, Y′ and Y″ are nitrogen and Z′ is a carbon or a nitrogen.

In some embodiments, the compounds of Formulae I, IIa, IIb, II, II′, II′a, II′b, II″, II′″, IIIa, IIIb, III, III′, IVa, Va, IV, and V contain an unsubstituted aliphatic group, a substituted aliphatic group, an unsubstituted aliphatic ester group, a substituted aliphatic ester group, an unsubstituted aliphatic ether group, a substituted aliphatic ether group, an unsubstituted heterocyclic-aliphatic group, a substituted heterocyclic-aliphatic group, an unsubstituted aliphatic-amide group, a substituted aliphatic-amide group, an unsubstituted aryl-aliphatic group, and/or a substituted aryl-aliphatic group, such as R₁, R₂, R₈, R₁′, R₂′, R₈′, R₄, R₅, R₆, R₇, R₄′, R₅′, R₆′, R₇′, R₂₂, R₂₂′, R₂₂″, R₂₂″, R₂₃, R₂₄, R₂₅, R₂₆, and/or R₂₇, where the aliphatic group of the unsubstituted aliphatic group, the substituted aliphatic group, the unsubstituted aliphatic ester group, the substituted aliphatic ester group, the unsubstituted aliphatic ether group, the substituted aliphatic ether group, the unsubstituted heterocyclic-aliphatic group, the substituted heterocyclic-aliphatic group, the unsubstituted aliphatic-amide group, the substituted aliphatic-amide group, the unsubstituted aryl-aliphatic group, and/or the substituted aryl-aliphatic group, is a C₁-C₁₄ aliphatic group, optionally a C₁-C₆ aliphatic group.

In some embodiments, the compounds have the structures of Formula IV′.

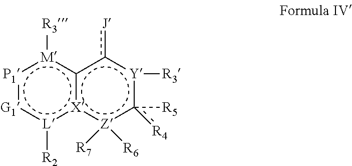

wherein X′, L′, G₁′, P₁′, M′, Y′, Z′, J′, R₃′, R₃′″, R₂, and R₄—R₇ are as defined above.

In some embodiments, the compounds have the structures of Formula VIa or VI′a.

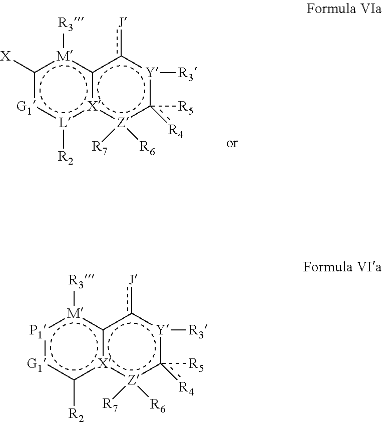

wherein X′, X, L′, G₁′, P₁′, M′, Y′, Z′, J′, R₃′, R₃′″, R₂, and R₄—R₇ are as defined above.

In some embodiments, the compounds have the structures of Formula VI, Formula VI′, Formula VI″, or Formula VI′″.

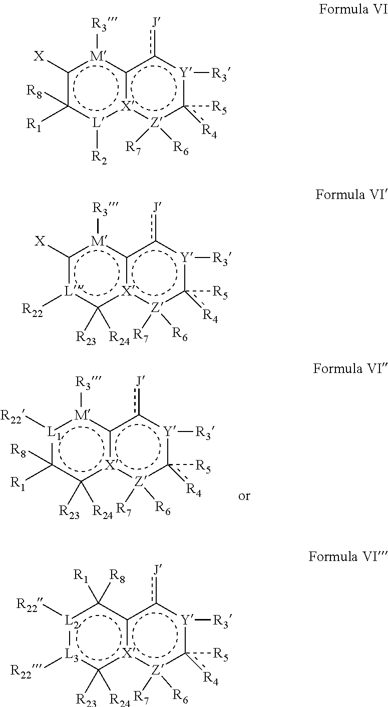

where X′, X, L′, L′″, L₁-L₃, M′, Y′, Z′, J′, R₃′, R₃′″, R₁-R₈, R₂₂—R₂₄, R₂₂′, R₂₂″, and R₂₂′″ are as defined above. In some embodiments, the compounds of Formula VI or VI′ are disclosed where X′, L′, L′″, M′, and Y′ are independently a carbon or a nitrogen, and Z′ is a carbon, an oxygen, or a nitrogen. In some embodiments, the compounds of Formula VI or VI′ are disclosed where X′ is carbon, L′, L′″, and M′ are independently a carbon or a nitrogen, Y′ is a nitrogen, and Z′ is an oxygen, a nitrogen, or a carbon. In some embodiments, the compounds of Formula VI″ or VI′″ are disclosed where X′, L₁, L₂, L₃, M′, and Y′ are independently a carbon or a nitrogen, and Z′ is a carbon, an oxygen, or a nitrogen. In some embodiments, the compounds of Formula VI″ or VI′″ are disclosed where X′ is carbon, L₁, L₂, L₃, and M′ are nitrogen, Y′ is a nitrogen, and Z′ is a nitrogen or a carbon.

In some embodiments, the compounds of Formula VI are disclosed where X′ is carbon and L′, M′, Y′, and Z′ are independently a carbon or a nitrogen. In some embodiments, the compounds of Formula VI are disclosed where X′ is carbon, L′, M′, and Y′ are nitrogen, and Z′ is independently an oxygen, a nitrogen, or a sulfur. In some embodiments, the compounds of Formula VI are disclosed where X′ is carbon, L′, M′, and Y′ are nitrogen, and Z′ is oxygen.

In some embodiments, the compounds are pyrazino-oxazine having the structures of Formula VII, Formula VII′, or Formula VII″.

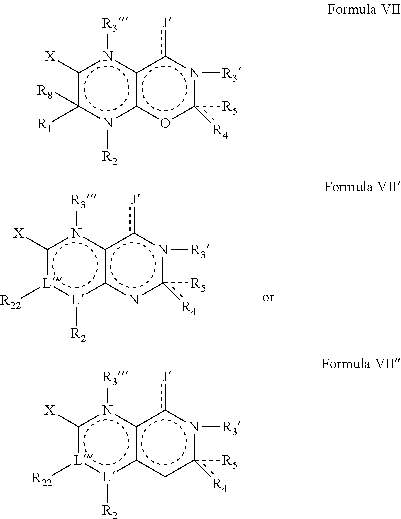

where X, R₃′, R₃′″, J′, R₁, R₂, R₄, R₅, R₈, and R₂₂ are as defined above; and where L′ and L′″ are independently a carbon or a nitrogen.

In some embodiments, the compounds are pyrazino-oxazine having the structures of Formula VIII, IX, or X.

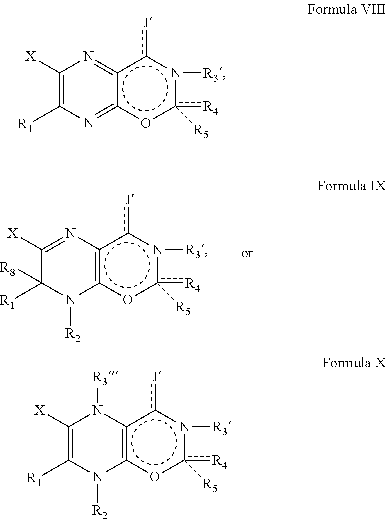

where X, R₃′, R₃′″, J′, R₁, R₂, R₄, R₅, and R₈ are as defined above.

In some embodiments, the compounds are triazolo-pyrazine having the structures of Formula XIa.

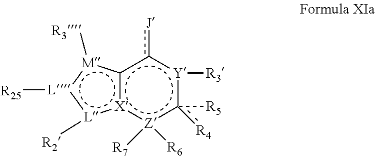

wherein X′, L″, L″″, M″, Z′, Y′, J′, R₂′, R₃′, R₃₉′″, R₈′, R₄—R₇, and R₂₅ are as defined above.

In some embodiments, the compounds are triazolo-pyrazine having the structures of Formula XI or Formula XI′.

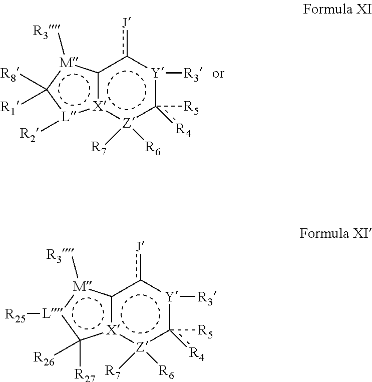

where X′, L″, L″″, M″, Z′, Y′, J′, R₁′—R₃′, R₃′″″, R₈′, R₄—R₇, and R₂₅—R₂₇ are as defined above.

In some embodiments, the compounds of Formula XI or Formula XI′ are disclosed where M″, L″, L″″, X′, Y′, and Z′ are independently a carbon or a nitrogen. In some embodiments, the compounds of Formula XI are disclosed where M″ and L″ are nitrogen, Z′ is carbon, and X′ and Y′ are independently a carbon or a nitrogen. In some embodiments, the compounds of Formula XI are disclosed where X′, M″, L″, and Y′ are nitrogen and Z′ is a carbon or a nitrogen.

In some embodiments, the compounds are triazolo-pyrazine having the structures of Formula XII or Formula XII′.

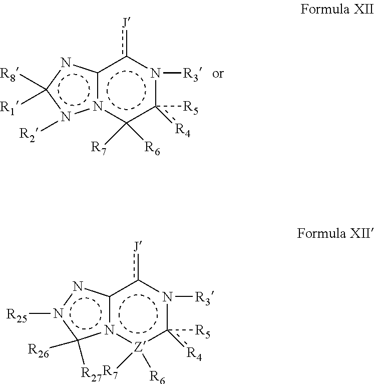

where J′, R₁′—R₃′, R₈′, R₄—R₇, and R₂₅—R₂₇ are as defined above; and where Z′ is a carbon or a nitrogen.

In some embodiments, the compounds are triazolo-pyrazine having the structures of Formulae XIII, XIII′, XIII″, XIV, or XV.

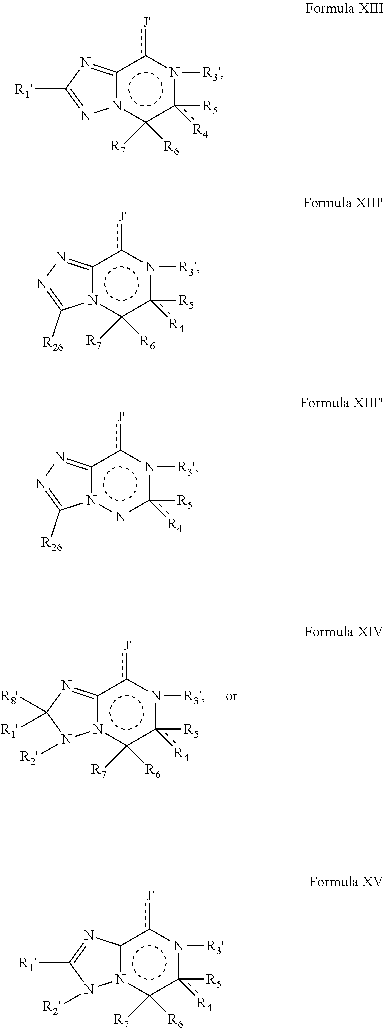

where J′, R₁′—R₃′, R₈′, R₄—R₇, and R₂₆ are as defined above.

In some embodiments, the compounds are triazolo-imidazole having the structures of Formula XVIa.

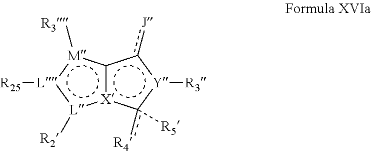

where X′, L″, L″″, M″, Y″, J″, R₂′, R₃″, R₃′″, R₄′, R₅′, and R₂₅ are as defined above.

In some embodiments, the compounds are triazolo-imidazole having the structures of Formula XVI or Formula XVI′.

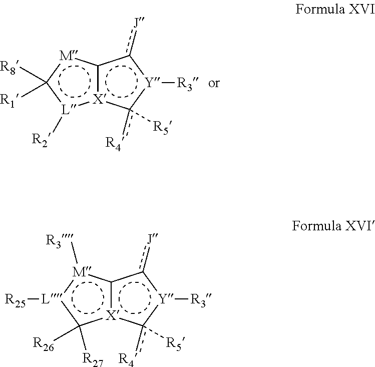

where X′, L″, L″″, M′, Y″, J″, R₂′, R₃′″, R₃″″, R₄′, R₈′, and R₂₅—R₂₇ are as defined above.

In some embodiments, the compounds of Formula XVI or Formula XVI′ are disclosed where L″, L″″, and M″ are independently a carbon or a nitrogen and X′ and Y″ are independently an oxygen, a nitrogen, or a sulfur. In some embodiments, the compounds of Formula XVI are disclosed where L″ and M″ are nitrogen and X′ and Y″ are independently an oxygen, a nitrogen, or a sulfur. In some embodiments, the compounds of Formula XVI are disclosed where X′, L″, and M″ are nitrogen and Y″ is an oxygen, a nitrogen, or a sulfur.

In some embodiments, the compounds are triazolo-imidazole having the structures of Formula XVII or Formula XVII′.

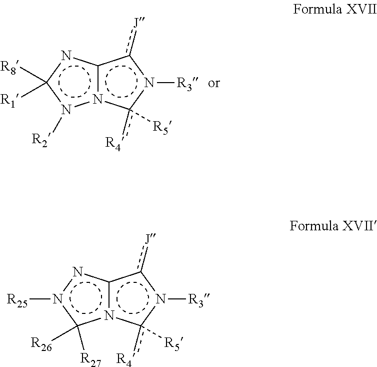

where J″, R₁′, R₂′, R₃″, R₄′, R₅′, R₈′ and R₂₅—R₂₇ are as defined above.

In some embodiments, the compounds are triazolo-imidazole having the structures of Formula XVIII, XIX, XX, or XX′.

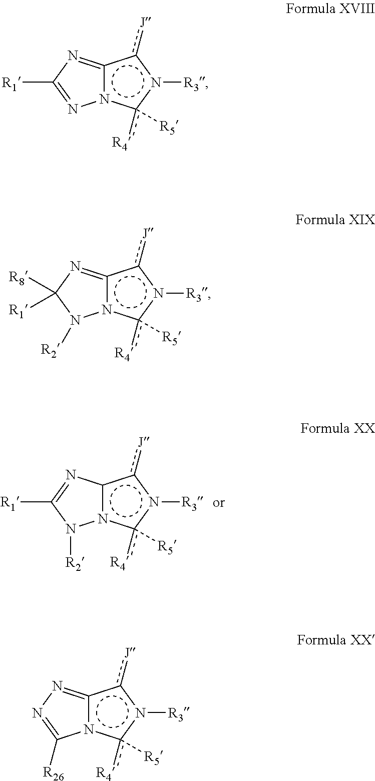

where J″, R₁′, R₂′, R₃″, R₄′, R₅′, R₈′, and R₂₆ are as defined above.

In some embodiments, the compounds of Formulae IV, IV′, V, VIa, VI, VIa, VI′, VI′a, VI″, VI′″, VII, VII′, VII″, VIII, IX, X, XIa, XI, XI′, XII, XII′, XIII, XIII′, XIII″, XIV, XV, XVIa, XVI, XVI′, XVII, XVII′, XVIII-XX, and XX′ are disclosed where J′ and J″ are independently an oxygen or OR₃, where R₃ is a hydrogen, an unsubstituted aliphatic group, a substituted aliphatic group, an unsubstituted aliphatic ester group, a substituted aliphatic ester group, an unsubstituted aliphatic ether group, a substituted aliphatic ether group, an unsubstituted heterocyclic group, a substituted heterocyclic group, an unsubstituted heterocyclic-aliphatic group, or a substituted heterocyclic-aliphatic group, and where the substituents are as defined above.

In some embodiments, the compounds have the structures of any one of Formulae 1-24 and 84-102 and tautomer thereof.

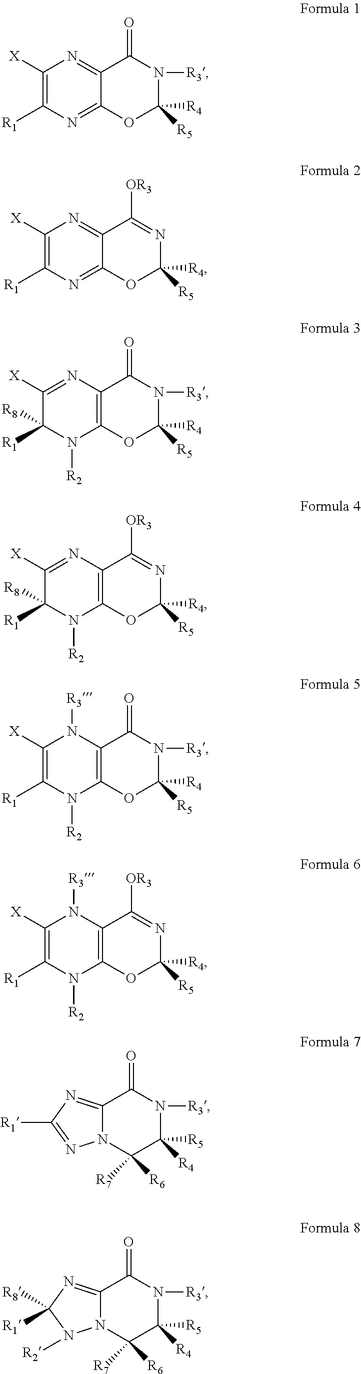

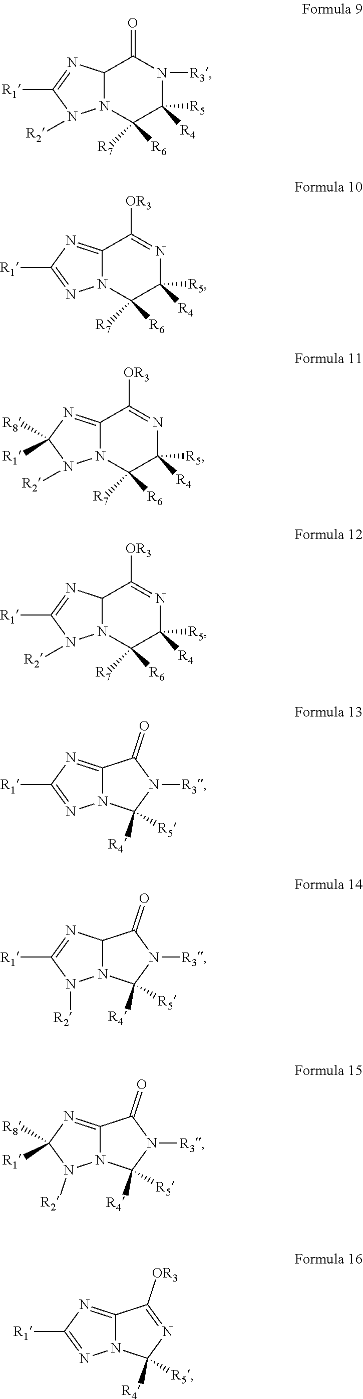

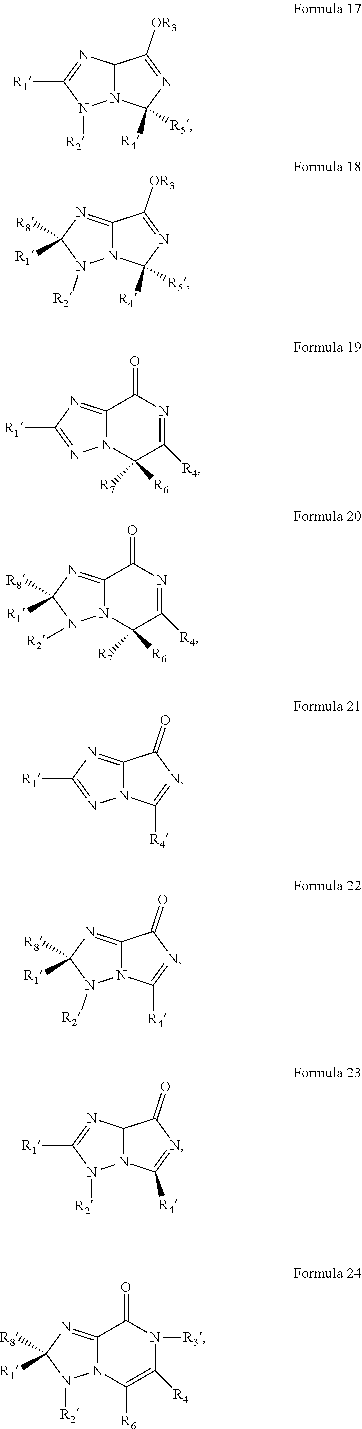

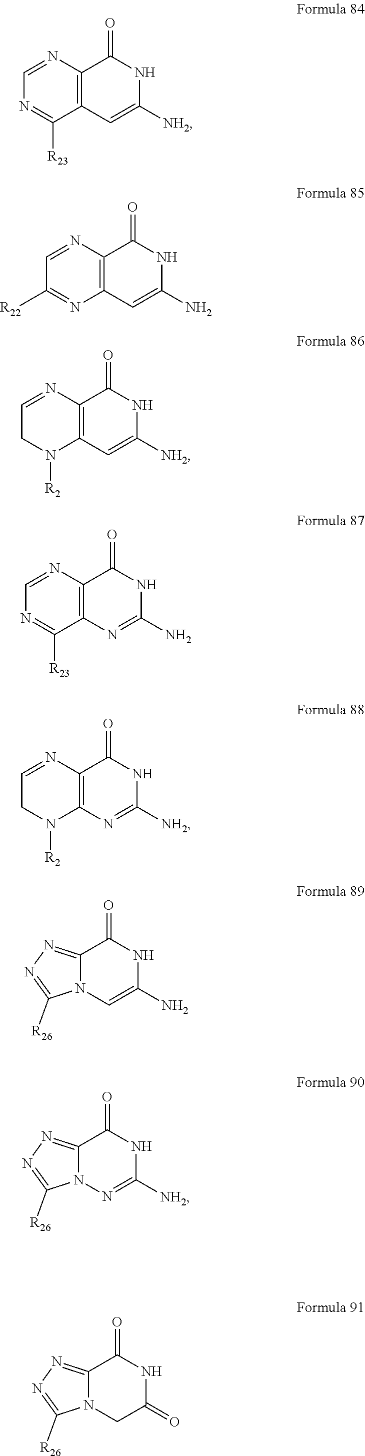

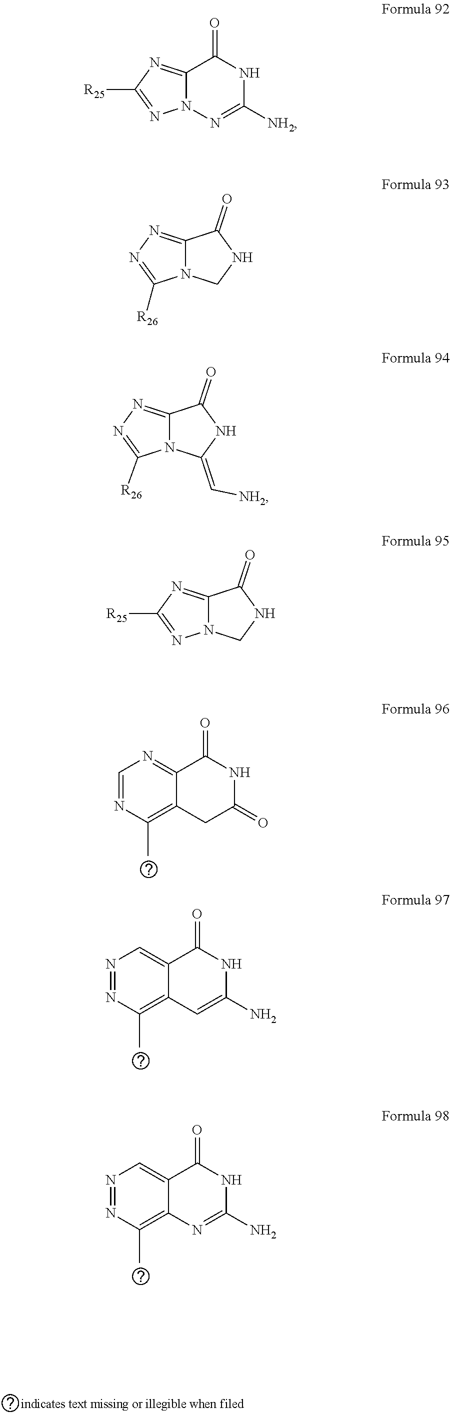

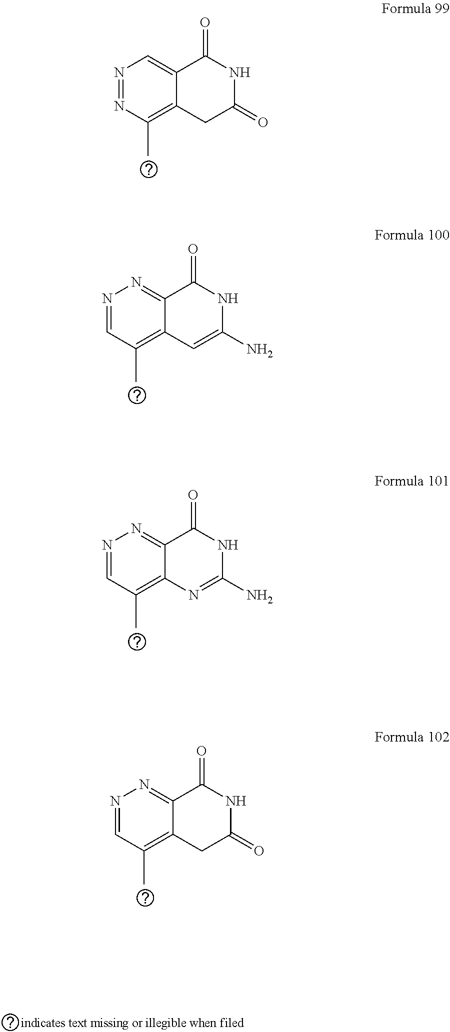

where X, R₁-R₈, R₂₂, R₂₃, R₂₅, R₂₆, R₁′—R₅′, R₈′, R₃″, and R₃′″ are as defined above.

In some embodiments, the aliphatic group of the compounds of any one of Formulae II, IIa, IIb, II′a, II′b, IIIa, IIIb, II, II′, II″, II′″, III, III′, IVa, Va, IV, IV′, V, VIa, VI′a, VI, VI′, VI″, VI′″, VII, VII′, VII″, VIII-X, XIa, XI, XI′, XII, XII′, XIII, XIII′, XIII″, XIV, XV, XVIa, XVI, XVI′, XVII, XVII′, XVIII-XX, XX′,1-24, and 84-102 is a C₁-C₁₄ aliphatic group, a C₁-C₁₂ aliphatic group, a C₁-C₁₀ aliphatic group, a C₁-C₈ aliphatic group, or a C₁-C₆ aliphatic group, or a combination thereof.

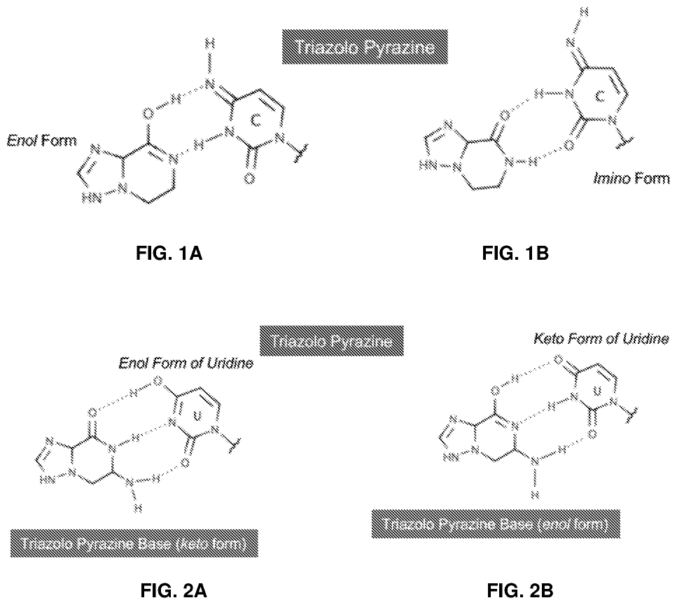

Typically, the compounds can form at least two hydrogen bonds with RNA nucleosides and hence show high potential to mimic RNA bases. For example, the compounds can form at least two hydrogen bonds or at least three hydrogen bonds with a RNA nucleoside, such as two hydrogen bonds or three hydrogen bonds. For example, when the compounds are triazolo-pyrazine having the structures of any one of Formulae XIa, XI, XI′, XII, XII′, XIII, XIII′, XIII″, XIV, XV, 7-12, 19-20, 24, and 89-92, the compounds can form at least two hydrogen bonds with cytidine (see, e.g. FIGS. 1A-1B). In some embodiments, when the compounds are pyrazino/pyrimidino/pyridazino oxazin having the structures of any one of Formulae VIa, VI′a, VI, VI′, VI″, VI′, VII, VII′, VII″, VIII-X, 1-6, 84-88, and 96-102 or are triazolo-imidazole having the structures of any one of Formulae XVIa, XVI, XVI′, XVII, XVII′, XVIII-XX, and XX′, 13-18, 21-23, and 93-95, the compounds can form at least two hydrogen bonds with cytidine. In some embodiments, R₁, R₂, R₈, R₁′, R₂′, R₈′, R₄, R₅, R₆, R₇, R₄′, R₅′, R₆′, R₇′, R₂₂, R₂₂′, R₂₂″, R₂₂″, R₂₃, R₂₄, R₂₅, R₂₆, and/or R₂₇ of any one of the Formulae described above is a nitrile group, an ester group, an unsubstituted aliphatic ester group, a substituted aliphatic ester group, an unsubstituted amino group, a substituted amino group, an amide group, an aliphatic-amide group, or an azide group. In such embodiments, the compounds can form at least three hydrogen bonds with a RNA nucleoside. For example, when the compounds are triazolo-pyrazine having the structures of any one of Formulae XIa, XI, XI′, XII, XII′, XIII, XIII′, XIII″, XIV, XV, 7-12, 19-20, 24, and 89-92, where R₄ or R₅ is an amino or oxo group, the compounds can form at least three hydrogen bonds with uridine (see, e.g. FIGS. 2A-2B). In some embodiments, when the compounds are pyrazino/pyrimidino/pyridazino oxazin having the structures of any one of Formulae VIa, VI′a, VI, VI′, VI″, VI′″, VII, VII′, VII″, VIII-X, 1-6, and 84-88, where R₄ or R₅ is an amino, an aliphatic-amino, or oxo group or when the compounds are triazolo-imidazole having the structures of any one of Formulae XVIa, XVI, XVI′, XVII, XVII′, XVIII-XX, and XX′, 13-18, 21-23, and 93-95, where R₄′ or R₅′ is an amino, an aliphatic-amino, or oxo group, the compounds can form at least three hydrogen bonds with cytidine.

In some embodiments, the compounds of any one of Formulae II, IIa, IIb, II′a, II′b, IIIa, IIIb, II, II′, II″, II′″, III, III′, IVa, Va, IV, IV′, V, VIa, VI′ a, VI, VI′, VI″, VI′″, VII, VII′, VII″, VIII-X, XIa, XI, XI′, XII, XII′, XIII, XIII′, XIII″, XIV, XV, XVIa, XVI, XVI′, XVII, XVII′, XVIII-XX, XX′,1-24, and 84-102 are disclosed, where the structures of the compounds are comparable to RNA nucleosides. For example, the compounds of any one of Formulae II, IIa, IIb, II′a, II′b, IIIa, IIIb, II, II′, II″, II′″, III, III′, IVa, Va, IV, IV′, V, VIa, VI′a, VI, VI′, VI″, VI′″, VII, VII′, VII″, VIII-X, XIa, XI, XI′, XII, XII′, XIII, XIII′, XIII″, XIV, XV, XVIa, XVI, XVI′, XVII, XVII′, XVIII-XX, XX′,1-24, and 84-102 are disclosed, where R₁, R₂, R₈, R₁′, R₂′, R₈′, R₄, R₅, R₆, R₇, R₄′, R₅′, R₆′, R₇′, R₂₂, R₂₂′, R₂₂″, R₂₂′″, R₂₃, R₂₄, R₂₅, R₂₆, and/or R₂₇ is an unsubstituted aliphatic ether or a substituted aliphatic ether, such that the structures of the compounds are comparable to RNA nucleosides. Compounds that are structurally comparable to RNA nucleosides (“nucleoside analogue prodrugs”) can act as competitive enzymatic inhibitors when incorporated into newly synthesized viral RNA.

The nucleoside analogue prodrugs may be a nucleoside or a nucleotide. The nucleoside analogue prodrugs can accelerate the absorption of these prodrugs into cells and the formation of the active metabolite drug, 5′ ribose triphosphate (RTP), which base-pairs with RNA bases and gets incorporated into the viral RNA strand, resulting in viral mutagenesis.

In some embodiments, the compounds are nucleoside analogue prodrugs, where R₁, R₂, R₈, R₁′, R₂′, R₈′, R₄, R₅, R₆, R₇, R₄′, R₅′, R₆′, R₇′, R₂₂, R₂₃, R₂₄, R₂₅, R₂₆, and/or R₂₇ of any one of Formulae II, IIa, IIb, II′a, II′b, IIIa, IIIb, II, II′, II′, II′″, III, III′, IVa, Va, IV, IV′, V, VIa, VI′a, VI, VI′, VI″, VI′″, VII, VII′, VII″, VIII-X, XIa, XI, XI′, XII, XII′, XIII, XIII′, XIII″, XIV, XV, XVIa, XVI, XVI′, XVII, XVII′, XVIII-XX, XX′,1-24, and 84-102 is an unsubstituted aliphatic ether or a substituted aliphatic ether, optionally an unsubstituted C₁-C₁₄ aliphatic ether or a substituted C₁-C₁₄ aliphatic ether, optionally an unsubstituted C₁-C₆ aliphatic ether or a substituted C₁-C₆ aliphatic ether, and where the substituents are as defined above.

In some embodiments, the compounds of any one of Formulae II, IIa, IIb, IIIa, IIIb, II′a, II′b, II, II′, II″, II′″, III, III′, IVa, Va, IV, IV′, V, VIa, VI′a, VI, VI′, VI″, VI′″, VII, VII′, VII″, VIII-X, XIa, XI, XI′, XII, XII′, XIII, XIII′, XIII″, XIV, XV, XVIa, XVI, XVI′, XVII, XVII′, XVIII-XX, XX′,1-24, and 84-102 are disclosed, where R₁, R₂, R₈, R₁′, R₂′, R₈′, R₄, R₅, R₆, R₇, R₄′, R₅′, R₆′, R₇′, R₂₂, R₂₃, R₂₄, R₂₅, R₂₆, and/or R₂₇ is a substituted C₁-C₆ aliphatic ether having the structure of Formula XXI.

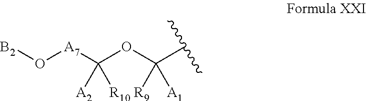

where A₁ and A₂ are independently a hydrogen, a halogen, a hydroxyl group, a thiol group, a cyanide group, a nitrile group, an unsubstituted aliphatic group, a substituted aliphatic group, an unsubstituted heterocyclic group, a substituted heterocyclic group, an unsubstituted aryl group, or a substituted aryl group;
where A₇ is a methylene group or an ethylene group;
where R₉ and R₁₀ are independently a hydrogen, a hydroxyl group, an unsubstituted alkyl group, or a substituted alkyl group, optionally wherein R₉ and R₁₀ are independently a substituted alkyl group that together form a ring; and
where B₂ is hydrogen or a phosphoramidate,
where the substituents are as defined above.

In some embodiments, the compounds of any one of Formulae II, IIa, IIb, II′a, II′b, IIIa, IIIb, II, II′, II″, II′″, III, III′, IVa, Va, IV, IV′, V, VIa, VI′a, VI, VI′, VI″, VI′″, VII, VII′, VII″, VIII-X, XIa, XI, XI′, XII, XII′, XIII, XIII′, XIII″, XIV, XV, XVIa, XVI, XVI′, XVII, XVII′, XVIII-XX, XX′,1-24, and 84-102 are disclosed, where R₁, R₂, R₈, R₁′, R₂′, R₈′, R₄, R₅, R₆, R₇, R₄′, R₅′, R₆′, R₇′, R₂₂, R₂₃, R₂₄, R₂₅, R₂₆, and/or R₂₇ has the structure of Formula 25 or 25′.

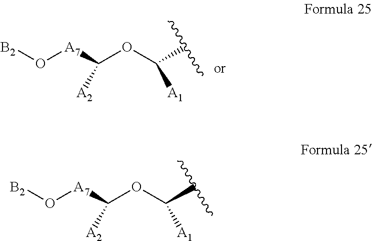

where A₁, A₂, A₇, and B₂ are as defined above.

In some embodiments, the compounds of any one of Formulae II, IIa, IIb, II′a, II′b, IIIa, IIIb, II, II′, II″, II′″, III, III′, IVa, Va, IV, IV′, V, VIa, VI′a, VI, VI′, VI″, VI′″, VII, VII′, VII″, VIII-X, XIa, XI, XI′, XII, XII′, XIII, XIII′, XIII″, XIV, XV, XVIa, XVI, XVI′, XVII, XVII′, XVIII-XX, XX′,1-24, and 84-102 are disclosed, where R₁, R₂, R₈, R₁′, R₂′, R₈′, R₄, R₅, R₆, R₇, R₄′, R₅′, R₆′, R₇′, R₂₂, R₂₃, R₂₄, R₂₅, R₂₆, and/or R₂₇ has the structure of Formula XXII.

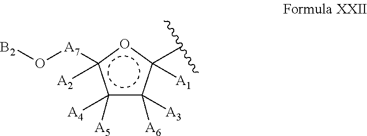

where A₁, A₂, A₇, and B₂ are as defined above; and
wherein A₃, A₄, A₅, and A₆ are independently absent, a hydrogen, a halogen, a hydroxyl group, a thiol group, an unsubstituted aliphatic group, a substituted aliphatic group, an unsubstituted heterocyclic group, a substituted heterocyclic group, an unsubstituted aryl group, or a substituted aryl group,
where the substituents are as defined above.

In some embodiments, the compounds of any one of Formulae II, IIa, IIb, II′a, II′b, IIIa, IIIb, II, II′, II″, II′″, III, III′, IVa, Va, IV, IV′, V, VIa, VI′a, VI, VI′, VI″, VI′″, VII, VII′, VII″, VIII-X, XIa, XI, XI′, XII, XII′, XIII, XIII′, XIII″, XIV, XV, XVIa, XVI, XVI′, XVII, XVII′, XVIII-XX, XX′,1-24, and 84-102 are disclosed, where R₁, R₂, R₈, R₁′, R₂′, R₈′, R₄, R₅, R₆, R₇, R₄′, R₅′, R₆′, R₇′, R₂₂, R₂₃, R₂₄, R₂₅, R₂₆, and/or R₂₇ has the structure of Formula 26 or 26′.

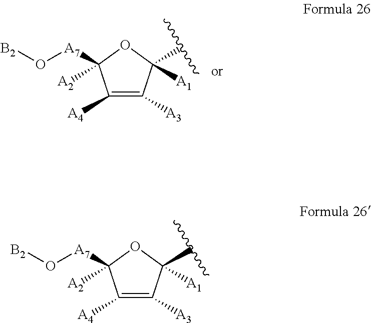

where A₇ and B₂ are as defined above.
where A₁ is an unsubstituted alkyl group, a substituted alkyl group, a cyanide group, a nitrile group, a hydroxyl group, or a halogen; and
where A₂, A₃, and A₄ are independently a hydrogen, a halogen, a hydroxyl group, a thiol group, an unsubstituted aliphatic group, a substituted aliphatic group, an unsubstituted heterocyclic group, a substituted heterocyclic group, an unsubstituted aryl group, or a substituted aryl group,
where the substituents are as defined above.

In some embodiments, the compounds of any one of Formulae II, IIa, IIb, II′a, II′b, IIIa, IIIb, II, II′, II″, II′″, III, III′, IVa, Va, IV, IV′, V, VIa, VI′a, VI, VI′, VI″, VI′″, VII, VII′, VII″, VIII-X, XIa, XI, XI′, XII, XII′, XIII, XIII′, XIII″, XIV, XV, XVIa, XVI, XVI′, XVII, XVII′, XVIII-XX, XX′,1-24, and 84-102 are disclosed, where R₁, R₂, R₈, R₁′, R₂′, R₈′, R₄, R₅, R₆, R₇, R₄′, R₅′, R₆′, R₇′, R₂₂, R₂₃, R₂₄, R₂₅, R₂₆, and/or R₂₇ has the structure of Formula 27 or 27′.

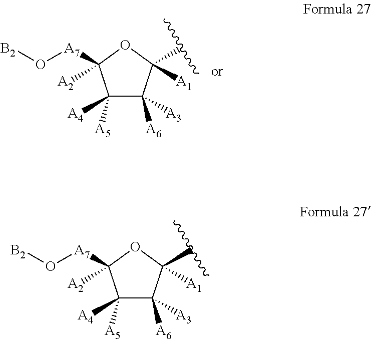

where A₇ and B₂ are as defined above.
where A₁ is an unsubstituted alkyl group, a substituted alkyl group, a cyanide group, a nitrile group, a hydroxyl group, or a halogen;
where A₂-A₆ are independently a hydrogen, a halogen, a hydroxyl group, a thiol group, an unsubstituted aliphatic group, a substituted aliphatic group, an unsubstituted heterocyclic group, a substituted heterocyclic group, an unsubstituted aryl group, or a substituted aryl group;
optionally where A₂, A₄, and A₆ are hydrogen, or A₁ is cyanide, or A₃ and/or A₆ are hydroxyl, or a combination thereof;
where the substituents are as defined above.

In some forms, Formula 27′ is

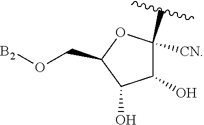

In some embodiments, the compounds of any one of Formulae II, IIa, IIb, II′a, II′b, IIIa, IIIb, II, II′, II″, II′″, III, III′, IVa, Va, IV, IV′, V, VIa, VI′a, VI, VI′, VI″, VI′″, VII, VII′, VII″, VIII-X, XIa, XI, XI′, XII, XII′, XIII, XIII′, XIII″, XIV, XV, XVIa, XVI, XVI′, XVII, XVII′, XVIII-XX, XX′,1-24, and 84-102 are disclosed, where R₁, R₂, R₁′, R₂, R₂₃, R₂₆, R₂₂, or R₂₅ has the structure of Formula XXI, Formula XXII, Formula 25, Formula 25′, Formula 26, Formula 26′, Formula 27, or Formula 27′.

In some embodiments, the compounds of any one of Formulae II, IIa, IIb, II′a, II′b, IIIa, IIIb, II, II′, II″, II′″, III, III′, IVa, Va, IV, IV′, V, VIa, VI′ a, VI, VI′, VI′″, VI′″, VII, VII′, VII″, VIII-X, XIa, XI, XI′, XII, XII′, XIII, XIII′, XIII″, XIV, XV, XVIa, XVI, XVI′, XVII, XVII′, XVIII-XX, XX′,1-27, and 84-102 are disclosed, where the structures of the compounds are comparable to RNA nucleotides. Compounds that are structurally comparable to RNA nucleosides can accentuate the antiviral activity of such compounds. For example, the compounds of any one of Formulae II, IIa, IIb, II′a, II′b, IIIa, IIIb, II, II′, II″, II′″, III, III′, IVa, Va, IV, IV′, V, VIa, VI′a, VI, VI′, VI″, VI′″, VII, VII′, VII″, VIII-X, XIa, XI, XI′, XII, XII′, XIII, XIII′, XIII″, XIV, XV, XVIa, XVI, XVI′, XVII, XVII′, XVIII-XX, XX′,1-27, and 84-102 are disclosed, where B₂ is hydrogen. For example, the compounds of any one of Formulae II, IIa, IIb, II′a, II′b, IIIa, IIIb, II, II′, II″, II′″, III, III′, IVa, Va, IV, IV′, V, VIa, VI′ a, VI, VI′, VI′″, VI′″, VII, VII′, VII″, VIII-X, XIa, XI, XI′, XII, XII′, XIII, XIII′, XIII″, XIV, XV, XVIa, XVI, XVI′, XVII, XVII′, XVIII-XX, XX′,1-27, and 84-102 are disclosed, where B₂ is a phosphoramidate having the structure of Formula 28.

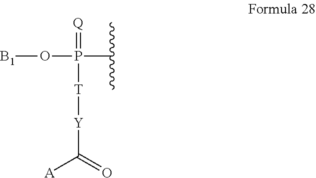

wherein B₁ is a hydrogen, an unsubstituted aliphatic group, a substituted aliphatic group, an unsubstituted aryl group, or a substituted aryl group;
wherein Q is an oxygen or a sulfur;
wherein T is an oxygen, a sulfur, NR₁₁, CR₁₁R₁₂, CR₁₁W₁, or CW₁W₂, wherein R₁₁ and R₁₂ are independently a hydrogen, an unsubstituted alkyl group, a substituted alkyl group, an unsubstituted aryl group, a substituted aryl group, and wherein W₁ and W₂ are independently a heteroatom;
wherein Y is an unsubstituted aliphatic group, a substituted aliphatic group, an unsubstituted heterocyclic group, a substituted heterocyclic group, an unsubstituted aryl group, or a substituted aryl group; and
wherein A is an unsubstituted aliphatic group, a substituted aliphatic group, an unsubstituted aryl group, a substituted aryl group, an alkoxy, —CM₁alkyl, —CM₁aryl, or -CM₁heterocyclic, wherein M₁ is an oxygen or a methylene group.

In some forms, A is —O-alkyl, where the alkyl can be a substituted or unsubstituted alkyl, such as an unsubstituted liner, branched, or cyclic C₁-C₆ alkyl, an unsubstituted liner, branched, or cyclic C₁-C₄ alkyl, or an unsubstituted liner, branched, or cyclic C₁-C₃ alkyl.

In some forms, B₁ is an unsubstituted aryl group or a substituted aryl group, such as an unsubstituted phenyl. In some forms, A-C(O)—Y-T- is

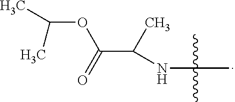 [SMILES: *NC(C)C(=O)OC(C)C]

In some embodiments, the compounds of any one of Formulae VI, VI′, VI″ and VI′″are disclosed, where R₁ or R₂ of Formula VI, R₂₂ or R₂₃ of Formula VI′, R₂₃ or R₁ of Formula VI″, and R₂₃, R₂₂″, or R₂₂′″ of Formula VI′″ has the structure of Formula XXI, Formula XXII, Formula 25, Formula 25′, Formula 26, Formula 26′, Formula 27, or Formula 27′; X and the other R groups are absent, hydrogen, substituted or unsubstituted aliphatic group, substituted or unsubstituted amino group, substituted or unsubstituted aliphatic-amino group, or oxo group (for example, R₄ or R₅ is an oxo or a substituted or unsubstituted amino or aliphatic-amino group and the rest of the R groups and X are independently substituted or unsubstituted aliphatic group, hydrogen or absent according to valency); and optionally B₂ has the structure of Formula 28. In some embodiments, the compounds of any one of Formulae XI and XI′ are disclosed, where R₁′ or R₂′ of Formula XI and R₂₅ or R₂₆ of Formula XI′ has the structure of Formula XXI, Formula XXII, Formula 25, Formula 25′, Formula 26, Formula 26′, Formula 27, or Formula 27′; the other R groups are absent, hydrogen, substituted or unsubstituted aliphatic group, substituted or unsubstituted amino group, substituted or unsubstituted aliphatic-amino group, or oxo group (for example, R₄ or R₅ is an oxo or a substituted or unsubstituted amino or aliphatic-amino group and the rest of the R groups are independently substituted or unsubstituted aliphatic group, hydrogen or absent according to valency); and optionally where B₂ has the structure of Formula 28. In some embodiments, the compounds of any one of Formulae XVI and XVI′ are disclosed, where R₁′ or R₂′ of Formula XVI and R₂₅ or R₂₆ of Formula XVI′ has the structure of Formula XXI, Formula XXII, Formula 25, Formula 25′, Formula 26, Formula 26′, Formula 27, or Formula 27′; the other R groups are absent, hydrogen, substituted or unsubstituted aliphatic group, substituted or unsubstituted amino group, substituted or unsubstituted aliphatic-amino group, or oxo group (for example, R₄ or R₅ is an oxo or a substituted or unsubstituted amino or aliphatic-amino group and the rest of the R groups are independently substituted or unsubstituted aliphatic group, hydrogen or absent according to valency); and optionally where B₂ has the structure of Formula 28. When these embodiments of Formulae VI, VI′, VI″, VI′″, XI, XI′, XVI and XVI′ are disclosed, J′ and J″ of any one of these Formulae can be independently an oxygen or OR₃, where R₃ is a hydrogen, a substituted or unsubstituted aliphatic group, or a substituted or unsubstituted amino group. The compounds may contain one or more chiral centers or may otherwise be capable of existing as multiple stereoisomers. These may be pure (single) stereoisomers or mixtures of stereoisomers, such as enantiomers, diastereomers, and enantiomerically or diastereomerically enriched mixtures. The compounds may be capable of existing as geometric isomers. Accordingly, it is to be understood that the present invention includes pure geometric isomers or mixtures of geometric isomers.

2. Exemplary Nucleoside Analogue Prodrugs and Nucleotides

Exemplary nucleoside analogue prodrugs can be prepared using three alternative C₁-C₆ aliphatic ethers of Formulae 26, 27, and 25 as R₁, R₂, R₁′, or R₂′ in compounds having the structures of Formulae 1-18. Exemplary nucleotides can be prepared by using phosphoramidates of Formula 28 as B₂ in the exemplary nucleoside analogue prodrugs.

It is understood that the exemplary compounds shown herein can exist in alternative stereoisomer forms. For example, the atoms/substituents attached to any one of the stereocenters of any one of the Formulae described in Table 1 below and the Examples below can have a different spatial configuration than those shown. For example, the aliphatic ether is attached to the bicyclic rings in an R configuration (see, e.g., Formulae 25′, 26′, and 27′). For example, the substituents on the cyclic ether have an R,R,S,R configuration. Preferably, when the bicyclic ring moiety of the disclosed compounds is a 6,6-bicyclic ring, the substituents on the cyclic ether attached to the 6,6-bicyclic ring have an R,R,S,R configuration.

It is also understood that the exemplary compounds shown herein can exist in alternative tautomer forms. For example, the bicyclic ring structure of each of the exemplary compounds described herein can present in the form of a tautomer or a mixture of two or three different forms of tautomer, such as a keto-keto form, a keto-enol form, an amino-enol form, an amino-keto form, or an enol-enol form, or a mixture thereof.

A summary of exemplary nucleoside analogue prodrugs and nucleotides that can be prepared is presented in Table 1. Although not illustrated in Table 1 below, it is understood that additional exemplary nucleoside analogue prodrugs and nucleotides can be prepared using the three alternative C₁-C₆ aliphatic ethers of Formulae 26, 27, and 25 as R₁′, R₂′, R₂, R₂₂, R₂₃, R₂₅, R₂₆ in compounds having any of the structures of Formulae 19-24 and 84-102.

TABLE 1
Exemplary nucleosides and nucleotides

| | | Nucleoside | |
|---|---|---|---|
| | | Analogue prodrug | |
| | | (R₁ or R′₁ is the | Nucleotide |
| | | aliphatic ether | (B₂ is a |
| | R₁, R₂, R₁′, or R₂′ is a C₁-C₆ | unless noted | phosphor- |
| Formula | Aliphatic Ether | otherwise) | amidate) |
| 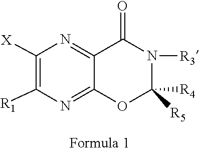 | 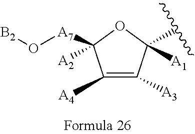 | 29a | 29b |
| | 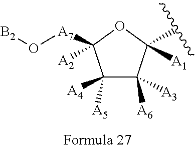 | 30a | 30b |
| | 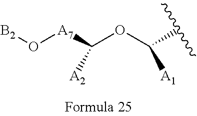 | 31a | 31b |
| 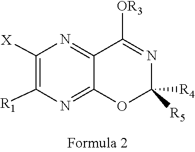 | 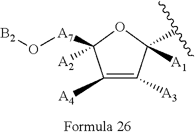 | 32a | 32b |
| | 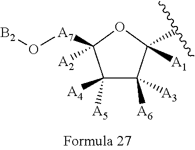 | 33a | 33b |
| | 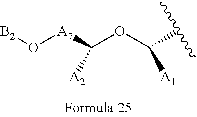 | 34a | 34b |
| 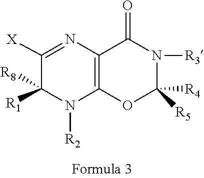 | 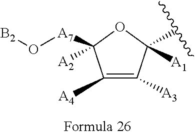 | 35a | 35b |
| | 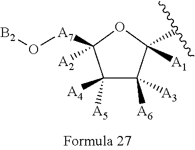 | 36a (R₂) | 36b (R₂) |
| | 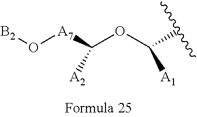 | 37a | 37b |
| 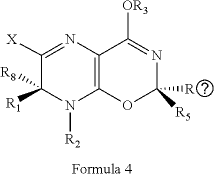 | 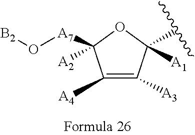 | 38a | 38b |
| | 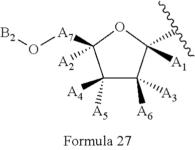 | 39a | 39b |
| | 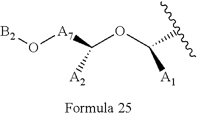 | 40a | 40b |
| 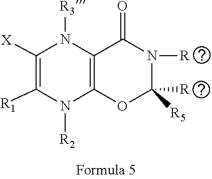 | 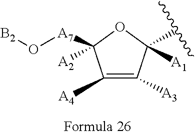 | 41a | 41b |
| | 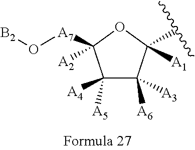 | 42a | 42b |
| | 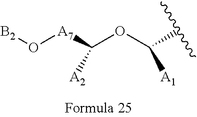 | 43a | 43b |
| 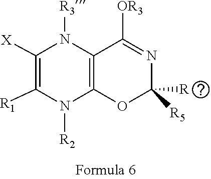 | 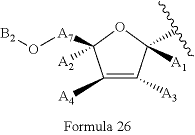 | 44a | 44b |
| | 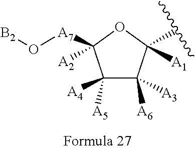 | 45a | 45b |
| | 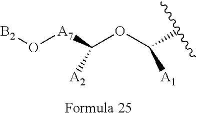 | 46a | 46b |
| 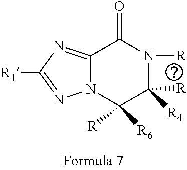 | 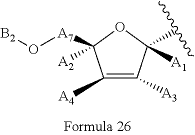 | 47a | 47b |
| | 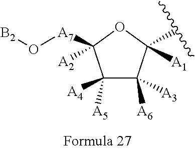 | 48a | 48b |
| | 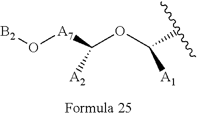 | 49a | 49b |
| 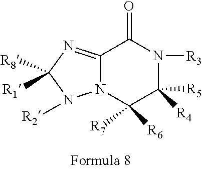 | 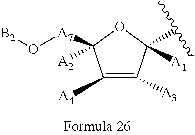 | 50a | 50b |
| | 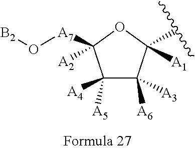 | 51a (R₂) | 51b (R₂) |
| | 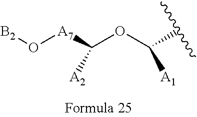 | 52a | 52b |
| 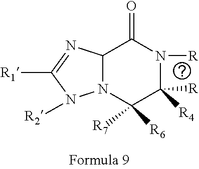 | 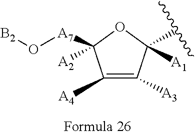 | 53a | 53b |
| | 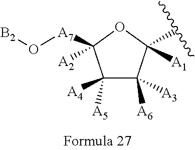 | 54a (R₂) | 54b (R₂) |
| | 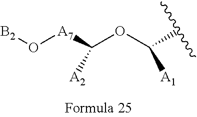 | 55a | 55b |
| 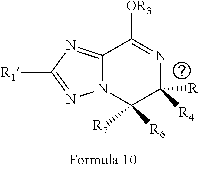 | 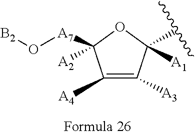 | 56a | 56b |
| | 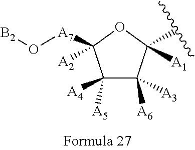 | 57a | 57b |
| | 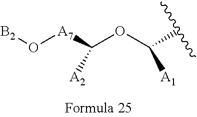 | 58a | 58b |
| 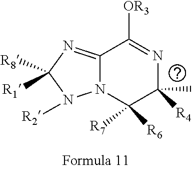 | 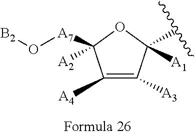 | 59a | 59b |
| | 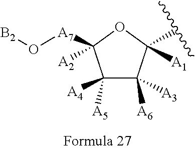 | 60a | 60b |
| | 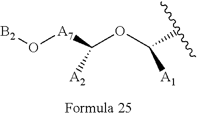 | 61a | 61b |
| 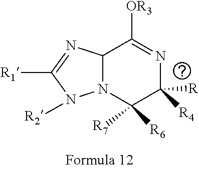 | 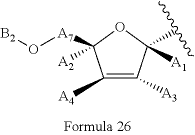 | 62a | 62b |
| | 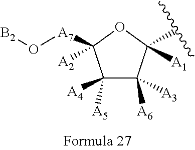 | 63a (R₂) | 63b (R₂) |
| | 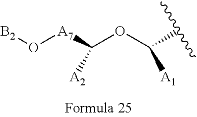 | 64a (R₂) | 64b (R₂) |
| 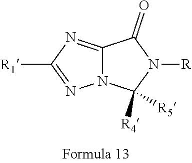 | 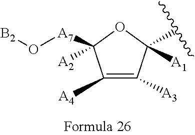 | 65a | 65b |
| | 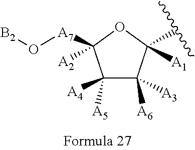 | 66a | 66b |
| | 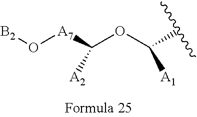 | 67a | 67b |
| 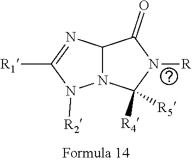 | 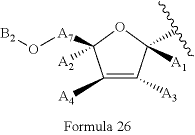 | 68a | 68b |
| | 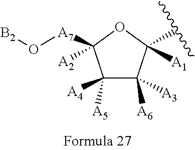 | 69a (R₂) | 69b (R₂) |
| | 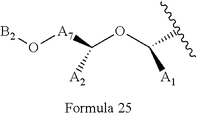 | 70a | 70b |
| 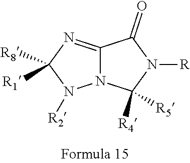 | 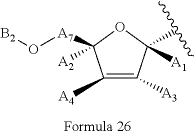 | 71a (R₂) | 71b (R₂) |
| | 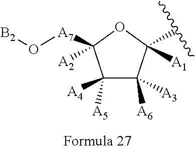 | 72a | 72b |
| | 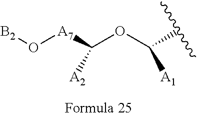 | 73a | 73b |
| 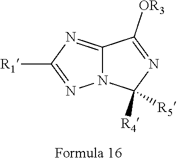 | 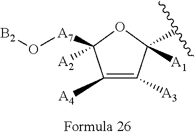 | 74a | 74b |
| | 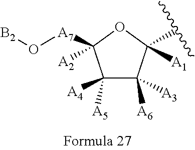 | 75a | 75b |
| | 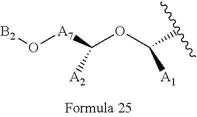 | 76a | 76b |
| 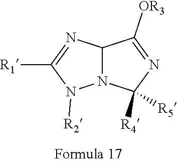 | 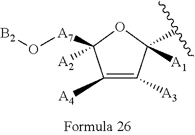 | 77a | 77b |
| | 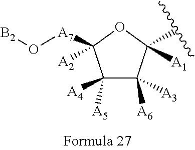 | 78a (R₂) | 78b (R₂) |
| | 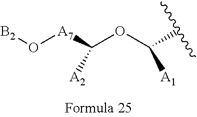 | 79a | 79b |
| 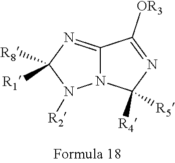 | 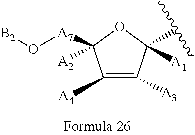 | 80a (R₂) | 80b (R₂) |
| | 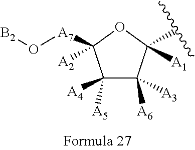 | 81a | 81b |
| | 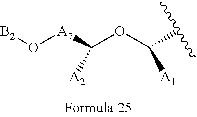 | 82a | 82b |

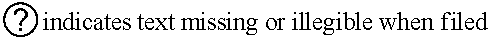

Specific exemplary compounds 29a-33b, 36a, 36b, 47a, 47b, 51a, 51b, 54a, 54b, 57a, 57b, 63a-65b, 69a, 69b, 71a, 71b, 75a, 75b, 78a, 78b, 81a, and 81b in Table 1 and additional exemplary compounds are described in the Examples section below.

In some embodiments, the compound has the structure of Formula 83 (also referred to therein as “T-101”) or Formula 83′ (also referred to therein as “T-101′”).

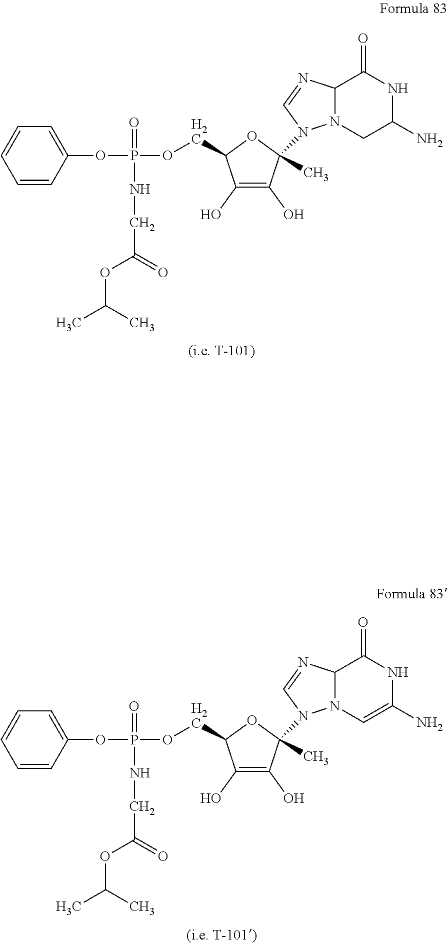 [SMILES: CC(C)OC(=O)CNP(=O)(OC[C@H]1O[C@](C)(N2C=NC3C(=O)NC(N)CN32)C(O)=C1O)Oc1ccccc1 ; CC(C)OC(=O)CNP(=O)(OC[C@H]1O[C@](C)(N2C=NC3C(=O)NC(N)=CN32)C(O)=C1O)Oc1ccccc1]

3. Pharmaceutically Acceptable Salts

The compounds may be neutral or may be one or more pharmaceutically acceptable salts, crystalline forms, non-crystalline forms, hydrates, or solvates, or a combination thereof. References to the compounds may refer to the neutral molecule, and/or those additional forms thereof collectively and individually from the context. Pharmaceutically acceptable salts of the compounds include the acid addition and base salts thereof.

Suitable acid addition salts are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

4. Effective Doses

Effective doses of the present compounds depend on many factors, including the indication being treated, the route of administration, co-administration of other therapeutic compositions, and the overall condition of the patient.

In general, treatment regimens utilizing compounds comprise administration of from about 0.1 mg to about 300 mg of the compounds per kilogram body weight of the recipient per day in multiple doses or in a single dose. In some embodiments, a suitable dose may be in the range of 0.1 to 300 mg per kilogram body weight of the recipient per day, optionally in the range of 6 to 150 mg per kilogram body weight per day, and optionally in the range 15 to 100 mg per kilogram body weight per day. The desired dose may be presented as two, three, four, five or six or more sub-doses administered at appropriate intervals throughout the day. These sub-doses may be administered in unit dosage forms, for example, containing 10 to 1500 mg, optionally 20 to 1000 mg, and optionally 50 to 700 mg of the compounds per unit dosage form.

B. Pharmaceutical Compositions

Pharmaceutical compositions and pharmaceutical formulations in unit dosage form suitable for the delivery of the compounds thereof and their preparation are disclosed. Generally, the pharmaceutical composition or formulation contains the compounds and/or the pharmaceutically acceptable salts of the compounds described herein, and a pharmaceutically acceptable excipient. The term “pharmaceutically acceptable excipient” is used herein to describe any ingredient in the formulation other than the compounds described herein. The pharmaceutical compositions or formulations can include an effective amount of one or more compounds of any of the formulae described herein and/or their pharmaceutically acceptable salts, including any one or any combination of compounds of the formulae described herein and/or their pharmaceutically acceptable salts, for preventing, treating, or ameliorating one or more symptoms associated with a viral infection in a subject. In some embodiments, the pharmaceutical composition or formulation can further contain one or more active agents in addition to the compounds, such as antiviral agents and/or anti-inflammatory agents. Antiviral agents and anti-inflammatory agents that can be included in the pharmaceutical compositions or formulations are known, for example, see Erik De Clercq, Medmicro, Chapter 52 (2000).

It is to be understood that combinations and/or mixtures of the compounds and/or their pharmaceutically acceptable salts may be included in the composition or formulation. In some embodiments, the pharmaceutical composition or formulation includes an effective amount of the compounds and/or their pharmaceutically acceptable salts for preventing, treating, or ameliorating one or more symptoms associated with a viral infection in a subject in a patient in need of relief.

Any one of more of the compounds provided herein can be expressly included or expressly excluded from the pharmaceutical compositions, dosage units, and/or methods of use or treatment disclosed herein.

1. Oral Formulations

The compounds and/or their pharmaceutically acceptable salts may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, powders, lozenges (including liquid-filled lozenges), chews, multi- and nano-particulates, gels, solid solutions, liposomes, films, ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose or a suitable oil, and one or more emulsifying agents and/or suspending agents. Liquid formulations may also be prepared by the reconstitution of a solid, for example, from a sachet.

The compounds and/or their pharmaceutically acceptable salts may also be used in fast-dissolving, fast-disintegrating dosage forms such as those described in Expert Opinion in Therapeutic Patents, 11 (6), 981-986, by Liang and Chen (2001).

For tablet or capsule dosage forms, depending on dose, the compounds and/or their pharmaceutically acceptable salts may make up from 1 weight % to 80 weight % of the dosage form, more typically from 5 weight % to 60 weight % of the dosage form. In addition to the compounds described herein, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (as, for example, the monohydrate, spray-dried monohydrate or anhydrous form), mannitol, xylitol, dextrose, sucrose, sorbitol, microcrystalline cellulose, starch and dibasic calcium phosphate dihydrate.

Tablets or capsules may also optionally contain surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets or capsules also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include glidants (e.g. Talc or colloidal anhydrous silica at about 0.1 weight % to about 3 weight %), anti-oxidants, colourants, flavouring agents, preservatives and taste-masking agents.

Exemplary tablets contain up to about 80% of one or more of the compounds described herein, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet or capsule blends may be compressed directly or by roller to form tablets. Tablet or capsule blends or portions of blends may alternatively be wet-, dry-, or melt-granulated, melt congealed, or extruded before tableting. The final formulation may contain one or more layers and may be coated or uncoated; it may even be encapsulated.

Solid formulations for oral administration may be formulated to be immediate and/or modified release. Modified release formulations include delayed, sustained, pulsed, controlled, targeted and programmed release formulations.

2. Parenteral Formulations

The compounds and/or their pharmaceutically acceptable salts may also be administered directly into the blood stream, into muscle, or into an internal organ. Suitable routes for such parenteral administration include intravenous, intraarterial, intraperitoneal, intrathecal, epidural, intracerebroventricular, intraurethral, intrasternal, intracranial, intramuscular, and subcutaneous delivery. Suitable means for parenteral administration include needle (including microneedle) injectors, needle-free injectors, and infusion techniques.

Parenteral formulations are typically aqueous solutions which may contain excipients such as salts, carbohydrates and buffering agents (preferably at a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of the compounds used in the preparation of a parenteral formulation may be increased by the use of appropriate formulation techniques, such as the incorporation of solubility-enhancing agents.

Formulations for parenteral administration may be formulated to be immediate and/or modified release. Modified release formulations include delayed, sustained, pulsed, controlled, targeted and programmed release formulations. Thus, the compounds may be formulated as a solid, semi-solid, or thixotropic liquid for administration as an implanted depot providing modified release of the active compound. Examples of such formulations include drug-coated stents and poly(dl-lactic-coglycolic)acid (PGLA) microspheres.

3. Pulmonary and Mucosal Formulations

The compounds and/or their pharmaceutically acceptable salts can be formulated for pulmonary or mucosal administration. The administration can include delivery of the composition to the lungs, nasal, oral (sublingual, buccal), vaginal, or rectal mucosa.

For example, the compounds can also be administered intranasally or by oral inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as water, ethanol-water mixture, 1,1,1,2-tetrafluoroethane or 1,1,1,2,3,3,3-heptafluoropropane. For intranasal or oral inhalation use, the powder may comprise a bioadhesive agent, for example, chitosan or cyclodextrin. The term aerosol as used herein refers to any preparation of a fine mist of particles, which can be in solution or a suspension, whether or not it is produced using a propellant. Aerosols can be produced using standard techniques, such as ultrasonication or high-pressure treatment.

The pressurized container, pump, spray, atomizer, or nebuliser contains a solution or suspension of one or more of the compounds including, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, a drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compounds described herein, a suitable powder base such as lactose or starch and a performance modifier such as 1-leucine, mannitol, or magnesium stearate. The lactose may be anhydrous or in the form of the monohydrate, preferably the latter. Other suitable excipients include dextran, glucose, maltose, sorbitol, xylitol, fructose, sucrose, and trehalose.

A suitable solution formulation for use in an atomizer using electrohydrodynamics to produce a fine mist may contain from 1 μg to 20 mg of one or more of the compounds per actuation and the actuation volume may vary from 1 μl to 100 μl. A typical formulation may contain one or more of the compounds described herein, propylene glycol, sterile water, ethanol and sodium chloride. Alternative solvents that may be used instead of propylene glycol include glycerol and polyethylene glycol.

Suitable flavors, such as menthol and levomenthol, or sweeteners, such as saccharin or saccharin sodium, may be added to those formulations intended for inhaled/intranasal administration.

Formulations for inhaled/intranasal administration may be formulated to be immediate and/or modified release using, for example, PGLA. Modified release formulations include delayed, sustained, pulsed, controlled, targeted, and programmed release formulations.

In the case of dry powder inhalers and aerosols, the dosage unit is determined by means of a valve which delivers a metered amount. Units in accordance with the compounds are typically arranged to administer a metered dose or “puff”. The overall daily dose will be administered in a single dose or, more usually, as divided doses throughout the day.

In some embodiments, the compounds and/or their pharmaceutically acceptable salts can be formulated for pulmonary delivery, such as intranasal administration or oral inhalation. Carriers for pulmonary formulations can be divided into those for dry powder formulations and for administration as solutions. Aerosols for the delivery of therapeutic agents to the respiratory tract are known in the art. For administration via the upper respiratory tract, the formulation can be formulated into an aqueous solution, e.g., water or isotonic saline, buffered or un-buffered, or as an aqueous suspension, for intranasal administration as drops or as a spray. Such aqueous solutions or suspensions may be isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. One skilled in the art can readily determine a suitable saline content and pH for an innocuous aqueous solution for nasal and/or upper respiratory administration.

In some embodiments, the aqueous solution is water, physiologically acceptable aqueous solutions containing salts and/or buffers, such as phosphate buffered saline (PBS), or any other aqueous solution acceptable for administration to an animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In some embodiments, solvents that are low toxicity organic (i.e. non-aqueous) class 3 residual solvents, such as ethanol, acetone, ethyl acetate, tetrahydrofuran, ethyl ether, and propanol may be used for the formulations. The solvent is selected based on its ability to readily aerosolize the formulation. The solvent should not detrimentally react with the compounds. An appropriate solvent should be used that dissolves the compounds or forms a suspension of the compounds. The solvent should be sufficiently volatile to enable formation of an aerosol of the solution or suspension. Additional solvents or aerosolizing agents, such as freons, can be added as desired to increase the volatility of the solution or suspension.

In some embodiments, the pharmaceutical compositions may contain minor amounts of polymers, surfactants, or other excipients well known to those of the art. In this context, “minor amounts” means no excipients are present that might affect or mediate uptake of the compounds by cells and that the excipients that are present in amount that do not adversely affect uptake of compounds by cells.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Non-aqueous suspensions of lipids can also be prepared in absolute ethanol using a reusable PARI LC Jet+ nebulizer (PARI Respiratory Equipment, Monterey, CA).

4. Topical Formulations

The compounds and/or their pharmaceutically acceptable salts may be administered directly to the external surface of the skin or the mucous membranes (including the surface membranes of the nose, lungs and mouth), such that the compounds and/or their pharmaceutically acceptable salts cross the external surface of the skin or mucous membrane and enters the underlying tissues.

Formulations for topical administration generally contain a dermatologically acceptable carrier that is suitable for application to the skin, has good aesthetic properties, is compatible with the active agents and any other components, and will not cause any untoward safety or toxicity concerns.

The carrier can be in a wide variety of forms. For example, emulsion carriers, including, but not limited to, oil-in-water, water-in-oil, water-in-oil-in-water, and oil-in-water-in-silicone emulsions, are useful herein. These emulsions can cover a broad range of viscosities, e.g., from about 100 cps to about 200,000 cps. These emulsions can also be delivered in the form of sprays using either mechanical pump containers or pressurized aerosol containers using conventional propellants. These carriers can also be delivered in the form of a mousse or a transdermal patch. Other suitable topical carriers include anhydrous liquid solvents such as oils, alcohols, and silicones (e.g., mineral oil, ethanol isopropanol, dimethicone, and cyclomethicone); aqueous-based single phase liquid solvents (e.g., hydro-alcoholic solvent systems, such as a mixture of ethanol and/or isopropanol and water); and thickened versions of these anhydrous and aqueous-based single phase solvents (e.g. where the viscosity of the solvent has been increased to form a solid or semi-solid by the addition of appropriate gums, resins, waxes, polymers, salts, and the like). Examples of topical carrier systems useful in the present formulations are described in the following references: “Sun Products Formulary” Cosmetics & Toiletries, vol. 105, pp. 122-139 (December 1990); “Sun Products Formulary,” Cosmetics & Toiletries, vol. 102, pp. 117-136 (March 1987); U.S. Pat. No. 5,605,894 to Blank et al., and U.S. Pat. No. 5,681,852 to Bissett.

Formulations for topical administration may be formulated to be immediate and/or modified release. Modified release formulations include delayed, sustained, pulsed, controlled, targeted and programmed release formulations. Thus, the compounds may be formulated as a solid, semi-solid, or thixotropic liquid for administration as an implanted depot providing modified release of the active compound. Examples of such formulations include drug-coated stents and poly(dl-lactic-coglycolic)acid (PGLA) microspheres.

### III. Methods of Use

A. Preventing, Treating, or Ameliorating Symptom(s) Associated with a Viral Infection

Methods of using the compounds for preventing, treating, or ameliorating one or more symptoms associated with a viral infection in a subject are disclosed.

Generally, the method includes (i) administering to the subject an effective amount of the compound(s) to prevent, treat, or ameliorate one or more symptoms associated with the viral infection in the subject. The subject can be a mammal. The compound(s) can be administered by a medical professional or the subject being treated (e.g. self-administration).

In some embodiments, the compounds and/or their pharmaceutically acceptable salts can be administered in the form of a pharmaceutical composition or formulation in association with one or more pharmaceutically acceptable excipients, such as the pharmaceutical composition or formulation described above. The choice of the pharmaceutically acceptable excipients will to a large extent depend on factors such as the particular mode of administration, the effect of the excipient on solubility and stability, and the nature of the dosage form.

Generally, the administered compounds can mimic DNA and/or RNA bases through base pairing with RNA and/or DNA nucleosides, thereby get incorporated into the viral RNA and/or DNA strand, resulting in viral mutagenesis (Strombaugh et al., Nucleic Acids Research, 37(7):2294-2312 (2009); Jin et al., POLS one, 8(7):e68347 (2013)). In particular, compounds that are structurally comparable to DNA and/or RNA nucleosides (also referred to herein as “nucleoside analogue prodrugs”) can act as competitive enzymatic inhibitors when incorporated into newly synthesized viral RNA and/or viral DNA. The nucleoside analogue prodrugs disclosed herein may be a nucleoside or a nucleotide. The nucleoside analogue prodrugs can accelerate the absorption of these prodrugs into cells and the formation of the active metabolite drug, 5′ ribose triphosphate (RTP), which base-pairs with DNA and/or RNA bases and gets incorporated into the viral DNA and/or viral RNA strand, resulting in viral mutagenesis (U.S. Pat. No. 7,018,989 to McGuigan et al.). For example, the exemplary compound T-101 can accelerate the absorption of T-101 into cells and the conversion of T-101 to T-101-5′-Ribosyl Triphosphate, which is the active metabolite.

1. Administration Routes

The compound(s) and/or their pharmaceutically acceptable salts or pharmaceutical composition or formulation containing the compound(s) and/or their pharmaceutically acceptable salts can be administered to the subject by oral administration, parenteral administration, inhalation, mucosal, topical administration, or a combination thereof.

For example, the compound(s) and/or their pharmaceutically acceptable salts or the pharmaceutical composition or formulation containing the compound(s) and/or their pharmaceutical acceptable slats can be orally administered to a subject by a medical professional or the subject being treated (e.g. self-administration). The compound(s) or the pharmaceutical composition or formulation containing the compound(s) and/or their pharmaceutical acceptable slats can be administered as tablets, capsules containing particulates, granules, powders, lozenges (including liquid-filled lozenges), chews, multi- and nano-particulates, gels, or liquids (e.g. solution or suspensions in aqueous or non-aqueous solvent).

Optionally, the compound(s) and/or their pharmaceutically acceptable salts or the pharmaceutical composition or formulation containing the compound(s) and/or their pharmaceutical acceptable slats can be administered to the subject by intravenous injection or intraperitoneal injection. The intravenous injection or intraperitoneal injection can be performed by a medical professional or the subject being treated (e.g. self-injection).

Alternatively, the compound(s) and/or their pharmaceutically acceptable salts or the pharmaceutical composition or formulation containing the compound(s) and/or their pharmaceutical acceptable slats can be administered to the subject by inhalation, such as mouth inhalation and/or nasal inhalation.

Optionally, the compound(s) and/or their pharmaceutically acceptable salts or the pharmaceutical composition or formulation containing the compound(s) and/or their pharmaceutical acceptable slats can be administered to the subject by topically applying the compound(s) or the pharmaceutical composition or formulation on one or more of the exposed surfaces of the subject.

2. Optional Steps

### a. Administering Additional Active Agent(s)

One or more active agents in addition to the compounds may be administered to the subject throughout the method or at different intervals during the method. For example, the one or more additional active agents is administered to the subject prior to, during, and/or subsequent to step (i). In some embodiments, the one or more additional active agents is included in a pharmaceutical composition or formulation containing the compound(s) and is administered to the subject simultaneously with the compound(s) in the pharmaceutical composition or formulation in association with one or more pharmaceutically acceptable excipients.

In some embodiments, the one or more additional active agents are one or more antiviral agents and/or one or more anti-inflammatory agents. Antiviral agents and anti-inflammatory agents are known in the art. The amount of the one or more antiviral agents and/or one or more anti-inflammatory agents required will vary from subject to subject according to their need.

3. Viruses Causing Viral Infections

The compounds will have broad antiviral properties and will be suitable for use in the prophylaxis and/or treatment of all classes of viruses.

For example, the compounds can be administered to a subject for preventing, treating, or ameliorating one or more symptoms associated with a viral infection in the subject caused by different classes of viruses. The viral infection in the subject to be treated may be caused by double stranded DNA viruses, single stranded DNA viruses, double stranded RNA viruses, single stranded positive sense RNA viruses, single stranded negative sense RNA viruses, positive sense single stranded RNA viruses that replicate through a DNA intermediate, double stranded DNA viruses that replicate through a single stranded RNA intermediate, single stranded RNA viruses with reverse transcriptase, or double stranded DNA viruses with reverse transcriptase, or a combination thereof. Exemplary viruses that can cause the viral infection in a subject include, but are not limited to, adenoviruses, herpesviruses, poxviruses, parvoviruses, SARS, Coronaviruses, Picornaviruses, Togaviruses, Influenza A viruses, Influenza B viruses, Influenza C viruses, Influenza D viruses, Retroviruses, Hepatitis B viruses, Hepatitis C viruses, or a combination thereof.

The amount of the compound(s) required will vary from subject to subject according to their need. Typically, the amount of the compound(s) is effective to prevent, treat, or ameliorate at least one of the one or more symptoms associated with the viral infection caused by any one of or a combination of the viruses described above.

Table 2 below summarizes the different viruses where the compounds can be used as prophylactic or treatment of viral infections.

TABLE 2
Viruses where the compounds can be used as prophylactic
or treatment of viral infections

| Class | Type of Virus | Examples of Viruses |
|---|---|---|
| I | dsDNA viruses | Adenoviruses, Herpesviruses, |
| | | Poxviruses |
| II | ssDNA viruses | parvoviruses |
| III | dsRNA viruses | adenovirus |
| IV | (+) ssRNA viruses [+strand or | SARS (e.g., SARS-CoV-2), |
| | sense] | Coronaviruses, Picornaviruses, |
| | | Togaviruses |
| V | (−) ssRNA viruses [−strand or | Influenza A (e.g., H1N1, |
| | antisense] | H3N2), B, C and D viruses |
| VI | ssRNA-RT viruses (+) sense | Retroviruses (e.g., HIV) |
| | RNA with DNA intermediate in | |
| | lifecycle | |
| VII | dsDNA-RT viruses | Hepatitis B and C viruses |

### a. Adenoviruses

In some embodiments, the subject being treated may have one or more symptoms associated with adenoviruses infection.

Adenoviruses are medium-sized (90-100 nm), non-enveloped icosahedral viruses with double-stranded DNA (dsDNA). More than 50 types of immunologically distinct adenoviruses can cause infections in humans. Most commonly adenoviruses cause respiratory illness, ranging from common cold to pneumonia, croup, and bronchitis. Depending on the type, adenoviruses can also cause other illnesses such as gastroenteritis, conjunctivitis, cystitis, and, less commonly, neurological disease. Immunosuppressed subjects are more susceptible and likely to develop more severe illness.

### b. Herpes Simplex Virus

In some embodiments, the subject being treated may have one or more symptoms associated with Herpes Simplex Virus (HSV) infection.

HSV is an infection that causes herpes. HSV is a common viral infection and affects all ages. Herpes can appear in various parts of the body, most commonly on the genitals or mouth. Two types of the herpes simplex virus exist, namely HSV-1, that primarily causes oral herpes, and is generally responsible for cold sores, fever blisters around the mouth and face and HSV-2 responsible for genital herpes outbreaks is primarily sexually transmitted.

### c. Poxviruses

In some embodiments, the subject being treated may have one or more symptoms associated with poxviruses infection.

Poxviruses are large double-stranded DNA (dsDNA) genomes. Variola virus (smallpox virus) emerged and began causing illness and deaths in human populations several thousand years ago, with smallpox outbreaks occurring occasionally.

### d. Parvovirus B19

In some embodiments, the subject being treated may have one or more symptoms associated with Parvovirus B19 infection.

Parvovirus B19 infects only humans. The infected human can have a range of symptoms depending on your age and overall health. Parvovirus B19 infection spreads through respiratory secretions, such as saliva, sputum, or nasal mucus, when an infected person coughs or sneezes, and can also spread through blood or blood products. Parvovirus B19 crosses the placenta and an infected pregnant woman can pass the virus to her baby.

### e. Severe Acute Respiratory Syndrome

In some embodiments, the subject being treated may have one or more symptoms associated with Severe acute respiratory syndrome (SARS).

SARS is a viral respiratory illness caused by a Coronavirus called SARS-associated coronavirus (SARS-CoV). Coronaviruses are (+) ssRNA associated with a nucleoprotein within a capsid comprised of matrix protein. The envelope bears club-shaped glycoprotein projections. Thought to originate from bats, these viruses are spread to humans through other animals. SARS-COV2 or COVID-19 is a recent pandemic infection that is now global and resulting in increased morbidity and mortality. The COVID-19 virus is known to affect the respiratory tract severely. The SARS -CoV-2—the virus behind the COVID-19 pandemic—enters the lungs, typically triggers an immune response, attracting immune cells to the region to attack the virus, resulting in localized inflammation. In some patients, excessive or uncontrolled levels of cytokines are released which then activate more immune cells, resulting in hyperinflammation-Cytokine storm, that can seriously harm or even kill the patient. Cytokine storms are a common complication not only of COVID-19 and flu but also other respiratory diseases caused by coronaviruses such as SARS and MERS. They are also associated with non-infectious diseases such as multiple sclerosis and pancreatitis. Favipiravir and Remdesivir two experimental viral polymerase inhibitors anecdotally are known to be effective in treating early-late stage coronavirus infections.

### f. Influenza Virus

In some embodiments, the subject being treated may have one or more symptoms associated with influenza virus infection.

Influenza virus includes seven genera and they are Influenza A, B, and C. Influenza A includes subtypes of H₁N1 (Spanish flu, swine flu), H₁N2, H₂N2 (Asian flu), H₃N1, H₃N2 (Hong Kong flu), H₃N8, H₅N1 (Avian or bird flu), H₅N2, H₅N3, H₅N8, H₅N9, H₇N1, H₇N2, H₇N3, H₇N4, H₇N7 (zoonotic potential), H₇N9, H₉N2, H₁₀N7. While humans are the common hosts of all three influenza viruses, animal avian hosts also are known to cause influenza infections (e.g., Avian flu caused by H₅N1). Influenza A viruses are classified based on the viral surface proteins hemagglutinin (HA or H) and neuraminidase (NA or N). More than sixteen H subtypes (or serotypes) and nine N subtypes of influenza A virus have been identified thus far. Every year new mutations of the influenza viruses are identified, and vaccines are prepared. However, the viral mutations quickly develop resistance and hence treatment becomes a more preferred line of therapy. Influenza A has a high level of morbidity and mortality. The compounds can be used to prevent and/or treat influenza infections in mammals.

### g. Retroviruses

In some embodiments, the subject being treated may have one or more symptoms associated with retroviruses infection.

Retroviruses are spherical viruses that contain RNA as their genetic material. Examples of retroviruses include human T cell leukemia virus, which causes cancer in humans, and the several types of human immunodeficiency virus (HIV), which is widely acknowledged to be the cause of acquired immunodeficiency syndrome (AIDS). Retroviruses are enveloped viruses with an RNA genome. The name is derived from the fact that the virus particle contains an RNA dependent DNA Polymerase (Reverse transcriptase). This enzyme converts the RNA genome into DNA, which then integrates into the host chromosomal DNA. The reverse transcriptase is highly error prone and rapid genetic variation is a feature of this group of viruses. Six human retroviruses have so far been identified. All of these infect T cells, HTLV 1—T-cell leukaemias/lymphomas, Tropical spastic paraparesis, HTLV 2 (unknown pathology), HIV 1 and 2 (AIDS). Two new human retroviruses were identified recently in Central Africa. They are related to HTLV1 and 2 and have been called HTLV 3 and 4.

### h. Hepatitis C

In some embodiments, the subject being treated may have one or more symptoms associated with Hepatitis C virus (HCV) infection.

HCV leads to serious and chronic liver conditions and/or diseases, such as cirrhosis and hepatocellular carcinoma, in a significant number of infected personnel. About 20% of chronically infected individuals eventually develop liver-destroying cirrhosis or cancer. The viral disease is transmitted parenterally by contaminated blood and blood products, contaminated needles, or sexually and vertically from infected mothers or carrier mothers to their offspring.

The HCV virion is an enveloped (+) RNA virus with a single oligoribonucleotide genomic sequence of about 9600 bases and which encodes a polyprotein of about 3,010 amino acids. The protein products of the HCV gene consist of the structural proteins C, E1, and E2, and the non-structural (NS) proteins NS2, NS3, NS4A and NS4B, and NS5A and NS5B. The NS proteins are believed to provide the catalytic process for viral replication. The NS3 protease releases NS5B, the RNA-dependent RNA polymerase from the polyprotein chain. HCV NS5B polymerase is required for the synthesis of a double-stranded RNA from a single-stranded viral RNA that serves as a template in the replication cycle of HCV. Therefore, NS5B polymerase is considered to be an essential component in the HCV replication complex (K. Ishi, et al. Hepatology, 29:1227-1235 (1999); V. Lohmann, et al, Virology, 249: 108-118 (1998)). Inhibition of HCV NS5B polymerase prevents formation of the double-stranded HCV RNA and therefore constitutes an attractive approach to the development of HCV-specific antiviral therapies.

The disclosed compounds and methods can be further understood through the following enumerated paragraphs.

1. A compound having the structure of

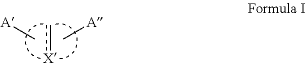

(a) wherein A′ and A″ are independently an unsubstituted heterocyclic group or a substituted heterocyclic group; and
(b) wherein X′ is a carbon or a nitrogen.
   2. The compound of paragraph 1, wherein the compound has the structure of

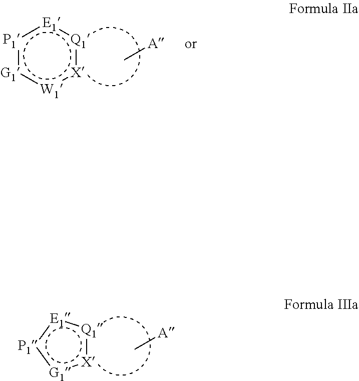

(a) wherein A″ are independently an unsubstituted heterocyclic group or a substituted heterocyclic group;
(b) wherein X′ is a carbon or a nitrogen;
(c) wherein E₁′, E₁″, P₁′, P₁″, G₁′, G₁″, and W₁′ are independently CR₂₀ or NR₂₁, and R₂₀ and R₂₁ are independently absent, a hydrogen, a halogen, a nitrile group, a nitro group, an oxo group, an ester group, an unsubstituted aliphatic ester group, a substituted aliphatic ester group, an ether group, an unsubstituted aliphatic ether group, a substituted aliphatic ether group, an unsubstituted amino group, a substituted amino group, an unsubstituted aliphatic amino group, a substituted aliphatic amino group, an amide group, an unsubstituted aliphatic-amide group, a substituted aliphatic-amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted heterocyclic group, a substituted heterocyclic group, an unsubstituted heterocyclic-aliphatic group, or a substituted heterocyclic-aliphatic group;
(d) wherein Q₁′ and Q₁″ are carbon; and
(e) wherein the substituents are independently a phosphoramidate, a heterocyclic group, a halogen, a sulfonic acid, an azide group, a cyanate group, an isocyanate group, a nitrate group, a nitrile group, an isonitrile group, a nitrosooxy group, a nitroso group, a nitro group, an aldehyde group, an alkoxy group, an acyl halide group, a carboxylic acid group, a carboxylate group, an alkyl group, a heteroalkyl group, an alkenyl group, a heteroalkenyl group, an alkynyl group, a heteroalkynyl group, an aryl group, a heteroaryl group, polyaryl group, a heteropoly aryl group, a alkyl aryl group, an amino group, an ester group, a hydroxyl group, a thiol group, a sulfonyl group, an ester group, an aliphatic ester group, an amide group, an aliphatic amide group, an azo group, an acyl group, a carbonyl group, a carbonate ester group, an ether group, an aminooxy group, or a hydroxyamino group.
   3. The compound of paragraph 1 or 2, wherein the compound has the structure of

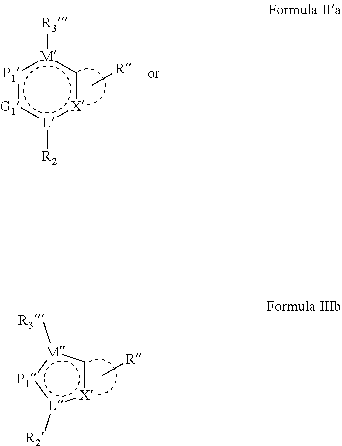

(a) wherein A″ are independently an unsubstituted heterocyclic group or a substituted heterocyclic group;
(b) wherein X′ is a carbon or a nitrogen;
(c) wherein L′, M′, L″, and M″ are independently a carbon or a nitrogen;
(d) wherein P₁′, P₁″, and G₁′ are independently CR₂₀ or NR₂₁, and R₂₀ and R₂₁ are independently absent, a hydrogen, a halogen, a nitrile group, a nitro group, an oxo group, an ester group, an unsubstituted aliphatic ester group, a substituted aliphatic ester group, an ether group, an unsubstituted aliphatic ether group, a substituted aliphatic ether group, an unsubstituted amino group, a substituted amino group, an unsubstituted aliphatic amino group, a substituted aliphatic amino group, an amide group, an aliphatic-amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted heterocyclic group, a substituted heterocyclic group, an unsubstituted heterocyclic-aliphatic group, or a substituted heterocyclic-aliphatic group;
(e) wherein R₂ and R₂′ are independently absent, a hydrogen, an unsubstituted aliphatic group, a substituted aliphatic group, an unsubstituted aryl group, a substituted aryl group, an unsubstituted aryl-aliphatic group, a substituted aryl-aliphatic group, an unsubstituted aliphatic ether group, or a substituted aliphatic ether group;
(f) wherein R₃′″ and R₃″″ are independently absent, a hydrogen, an unsubstituted aliphatic group, a substituted aliphatic group, an unsubstituted amide group, a substituted amide group, an unsubstituted aliphatic ester group, a substituted aliphatic ester group, an unsubstituted aliphatic ether group, a substituted aliphatic ether group, an unsubstituted heterocyclic group, a substituted heterocyclic group, an unsubstituted heterocyclic-aliphatic group, or a substituted heterocyclic-aliphatic group; and
(g) wherein the substituents are as defined in the base paragraph(s).
   4. The compound of any one of paragraphs 1-3, wherein the compound has the structure of

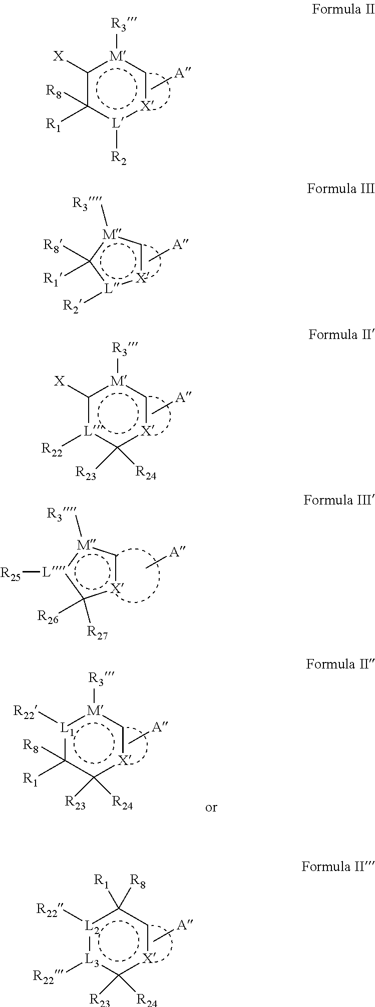

(a) wherein A″ are independently an unsubstituted heterocyclic group or a substituted heterocyclic group;
(b) wherein X′ is a carbon or a nitrogen;
(c) wherein L′, M′, L″, M″, L′″, L″″, and L₁-L₃ are independently a carbon or a nitrogen;
(d) wherein X is a hydrogen, a halogen, a nitrile group, a nitro group, an ester group, an unsubstituted aliphatic ester group, a substituted aliphatic ester group, an unsubstituted amino group, a substituted amino group, an amide group, an unsubstituted aliphatic-amide group, a substituted aliphatic-amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted heterocyclic group, a substituted heterocyclic group, an unsubstituted heterocyclic-aliphatic group, or a substituted heterocyclic-aliphatic group;
(e) wherein R₁, R₁′, R₂₃, and R₂₆ are independently a hydrogen, a halogen, a nitrile group, a nitro group, an ester group, an unsubstituted aliphatic ester group, a substituted aliphatic ester group, an unsubstituted amino group, a substituted amino group, an unsubstituted aliphatic amino group, a substituted aliphatic amino group, an amide group, an unsubstituted aliphatic amide group, a substituted aliphatic amide group, an azide group, an unsubstituted aliphatic group, a substituted aliphatic group, an unsubstituted aryl group, a substituted aryl group, an unsubstituted aryl-aliphatic group, a substituted aryl-aliphatic group, an ether group, an unsubstituted aliphatic ether group, a substituted aliphatic ether group;
(f) wherein R₂, R₂′, R₂₂, R₂₂′, R₂₂″, R₂₂″, and R₂₅ are independently absent, a hydrogen, an unsubstituted aliphatic group, a substituted aliphatic group, an unsubstituted aryl group, a substituted aryl group, an unsubstituted aryl-aliphatic group, a substituted aryl-aliphatic group, an unsubstituted aliphatic ether group, or a substituted aliphatic ether group;
(g) wherein R₃′″ and R₃″″ are independently absent, a hydrogen, an unsubstituted aliphatic group, a substituted aliphatic group, an unsubstituted amide group, a substituted amide group, an unsubstituted aliphatic ester group, a substituted aliphatic ester group, an unsubstituted aliphatic ether group, a substituted aliphatic ether group, an unsubstituted heterocyclic group, a substituted heterocyclic group, an unsubstituted heterocyclic-aliphatic group, or a substituted heterocyclic-aliphatic group;
(h) wherein R₈, R₈′, R₂₄, and R₂₇ are independently absent, a hydrogen, a halogen, a nitrile group, an ester group, an unsubstituted aliphatic ester group, a substituted aliphatic ester group, an unsubstituted amino group, a substituted amino group, an unsubstituted aliphatic amino group, a substituted aliphatic amino group, an amide group, a substituted aliphatic-amide group, an unsubstituted aliphatic-amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted aliphatic ether group, a substituted aliphatic ether group, un unsubstituted heterocyclic group, a substituted heterocyclic group, an unsubstituted heterocyclic-aliphatic group, or a substituted heterocyclic-aliphatic group; and
(i) wherein the substituents are as defined in the base paragraph(s).
   5. The compound of any one of paragraphs 1-4, wherein A″ is

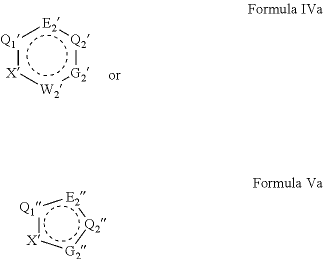

(a) wherein X′ is a carbon or a nitrogen;
(b) wherein E₂′, E₂″, G₂′, G₂″, Q₂′, Q₂″, and W₂′ are independently an oxygen, a sulfur, CR₂₀, or NR₂₁, and R₂₀ and R₂₁ are independently absent, a hydrogen, a halogen, a nitrile group, a nitro group, an oxo group, an ester group, an unsubstituted aliphatic ester group, a substituted aliphatic ester group, an ether group, an unsubstituted aliphatic ether group, a substituted aliphatic ether group, an unsubstituted amino group, a substituted amino group, an unsubstituted aliphatic amino group, a substituted aliphatic amino group, an amide group, an unsubstituted aliphatic-amide group, a substituted aliphatic-amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted heterocyclic group, a substituted heterocyclic group, an unsubstituted heterocyclic-aliphatic group, or a substituted heterocyclic-aliphatic group;
(c) wherein Q₁′ and Q₁″ are carbon; and
(d) wherein the substituents are as defined in the base paragraphs.
   6. The compound of any one of paragraphs 1-5, wherein A″ is

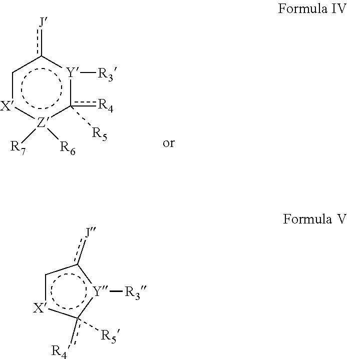

(a) wherein X′ is a carbon or a nitrogen;
(b) wherein Y′ and Y″ are nitrogen;
(c) wherein Z′ is a carbon, an oxygen, a nitrogen, or a sulfur;
(d) wherein J′ and J″ are independently an oxygen, OR₃, a sulfur, a thiol group, an unsubstituted amino group, or a substituted amino group,
(e) wherein R₃, R₃′, and R₃″ are independently absent, a hydrogen, an unsubstituted aliphatic group, a substituted aliphatic group, an unsubstituted aliphatic ester group, a substituted aliphatic ester group, an unsubstituted aliphatic ether group, a substituted aliphatic ether group, an unsubstituted heterocyclic group, a substituted heterocyclic group, an unsubstituted heterocyclic-aliphatic group, or a substituted heterocyclic-aliphatic group;
(f) wherein R₄, R₅, R₄′, R₅′, R₆, and R₇ are independently absent, a hydrogen, a halogen, a nitrile group, an oxo group, an ester group, an unsubstituted aliphatic ester group, a substituted aliphatic ester group, an unsubstituted amino group, a substituted amino group, an unsubstituted aliphatic-amino group, a substituted aliphatic-amino group, an amide group, an unsubstituted aliphatic-amide group, a substituted aliphatic-amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted aliphatic ether group, a substituted aliphatic ether group, un unsubstituted heterocyclic group, a substituted heterocyclic group, an unsubstituted heterocyclic-aliphatic group, or a substituted heterocyclic-aliphatic group; and
(g) wherein the substituents are as defined in the base paragraph(s).
   7. The compound of any one of paragraphs 1-6, wherein the compound has the structure of

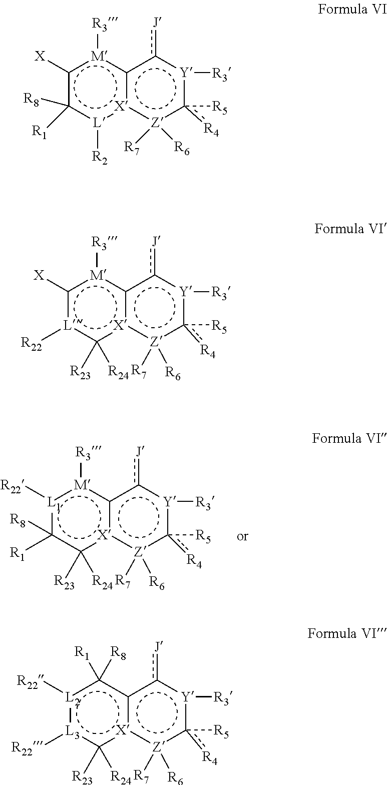

(a) wherein X′ is a carbon or a nitrogen;
(b) wherein L′, M′, L′″, and L₁-L₃ are carbon or nitrogen;
(c) wherein X is a hydrogen, a halogen, a nitrile group, a nitro group, an ester group, an unsubstituted aliphatic ester group, a substituted aliphatic ester group, an unsubstituted amino group, a substituted amino group, an amide group, an unsubstituted aliphatic-amide group, a substituted aliphatic-amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted heterocyclic group, a substituted heterocyclic group, an unsubstituted heterocyclic-aliphatic group, or a substituted heterocyclic-aliphatic group;
(d) wherein Y′ is a nitrogen;
(e) wherein Z′ is a an oxygen, a nitrogen, or a carbon;
(f) wherein J′ is an oxygen or OR₃;
(g) wherein R₁ and R₂₃ are independently a hydrogen, a halogen, a nitrile group, a nitro group, an ester group, an unsubstituted aliphatic ester group, a substituted aliphatic ester group, an unsubstituted amino group, a substituted amino group, an unsubstituted aliphatic amino group, a substituted aliphatic amino group, an amide group, an unsubstituted aliphatic amide group, a substituted aliphatic amide group, an azide group, an unsubstituted aliphatic group, a substituted aliphatic group, an unsubstituted aryl group, a substituted aryl group, an unsubstituted aryl-aliphatic group, a substituted aryl-aliphatic group, an ether group, an unsubstituted aliphatic ether group, a substituted aliphatic ether group;
(h) wherein R₂, R₂₂, R₂₂′, R₂₂″, and R₂₂′″ are independently absent, a hydrogen, an unsubstituted aliphatic group, a substituted aliphatic group, an unsubstituted aryl group, a substituted aryl group, an unsubstituted aryl-aliphatic group, a substituted aryl-aliphatic group, an unsubstituted aliphatic ether group, or a substituted aliphatic ether group;
(i) wherein R₃, R₃′, and R₃′″ are independently absent, a hydrogen, an unsubstituted aliphatic group, a substituted aliphatic group, an unsubstituted aliphatic ester group, a substituted aliphatic ester group, an unsubstituted aliphatic ether group, a substituted aliphatic ether group, an unsubstituted heterocyclic group, a substituted heterocyclic group, an unsubstituted heterocyclic-aliphatic group, or a substituted heterocyclic-aliphatic group;
(j) wherein R₈ and R₂₄ are independently absent, a hydrogen, a halogen, a nitrile group, an ester group, an unsubstituted aliphatic ester group, a substituted aliphatic ester group, an unsubstituted amino group, a substituted amino group, an unsubstituted aliphatic amino group, a substituted aliphatic amino group, an amide group, a substituted aliphatic-amide group, an unsubstituted aliphatic-amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted aliphatic ether group, a substituted aliphatic ether group, un unsubstituted heterocyclic group, a substituted heterocyclic group, an unsubstituted heterocyclic-aliphatic group, or a substituted heterocyclic-aliphatic group; and
(k) wherein R₄, R₅, R₆, and R₇ are independently absent, a hydrogen, a halogen, a nitrile group, an oxo group, an ester group, an unsubstituted aliphatic ester group, a substituted aliphatic ester group, an unsubstituted amino group, a substituted amino group, an unsubstituted aliphatic-amino group, a substituted aliphatic-amino group, an amide group, an unsubstituted aliphatic-amide group, a substituted aliphatic-amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted aliphatic ether group, a substituted aliphatic ether group, un unsubstituted heterocyclic group, a substituted heterocyclic group, an unsubstituted heterocyclic-aliphatic group, or a substituted heterocyclic-aliphatic group;
(J) wherein the substituents are as defined in the base paragraph(s).
   8. The compound of paragraph 7, wherein R₁ or R₂ of Formula VI, R₂₂ or R₂₃ of Formula VI′, R₂₃ or R₁ of Formula VI″, and R₂₃, R₂₂″, or R₂₂′″ of Formula VI′″ is a substituted an unsubstituted aliphatic ester group, a substituted aliphatic ester group, an unsubstituted aliphatic ether group, or a substituted aliphatic ether group, preferably an unsubstituted or substituted aliphatic ether, such as an unsubstituted C₁-C₁₄ aliphatic ether or a substituted C₁-C₁₄ aliphatic ether, or an unsubstituted C₁-C₆ aliphatic ether or a substituted C₁-C₆ aliphatic ether.
   9. The compound of paragraph 7 or 8, wherein R₄ and R₅ are independently absent, hydrogen, an oxo group, an unsubstituted amino group, a substituted amino group, an unsubstituted aliphatic-amino group, or a substituted aliphatic-amino group.
   10. The compound of any one of paragraphs 7-9, wherein when J′ is OR₃, R₃ is a hydrogen, a substituted or unsubstituted aliphatic group, or a substituted or unsubstituted amino group.
   11. The compound of any one of paragraphs 7-10, wherein L′, M′, L′″, and L₁-L₃ are nitrogen; X′ is carbon; Z′ is a nitrogen or a carbon; and R₆ and R₇ are independently absent or hydrogen.
   12. The compound of paragraph 7, wherein the compound has the structure of

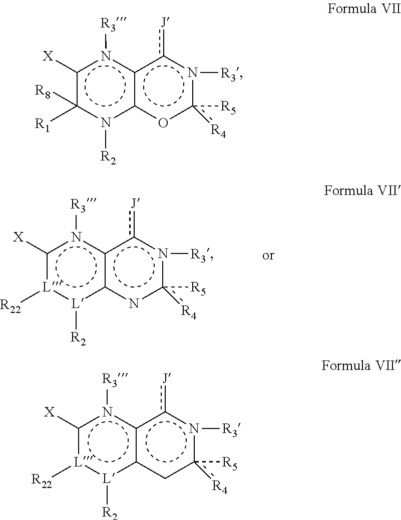

13. The compound of paragraph 11, wherein the compound has the structure of

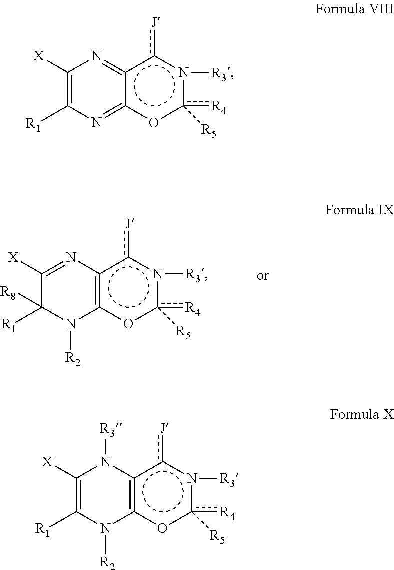

14. The compound of any one of paragraphs 1-6, wherein the compound has the structure of

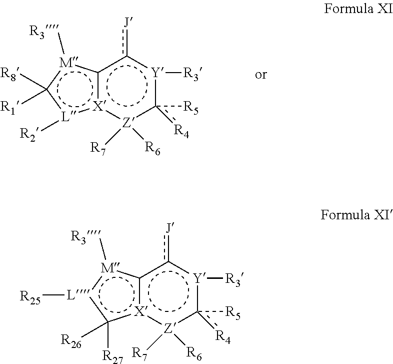

(a) wherein X′ is a carbon or a nitrogen;
(b) wherein L″, M″, and L′″″ are independently a carbon or a nitrogen;
(c) wherein Y′ is a nitrogen;
(d) wherein Z′ is a an oxygen, a nitrogen, or a carbon;
(e) wherein J′ is an oxygen or OR₃;
(f) wherein R₁′ and R₂₆ are independently a hydrogen, a halogen, a nitrile group, a nitro group, an ester group, an unsubstituted aliphatic ester group, a substituted aliphatic ester group, an unsubstituted amino group, a substituted amino group, an unsubstituted aliphatic amino group, a substituted aliphatic amino group, an amide group, an unsubstituted aliphatic amide group, a substituted aliphatic amide group, an azide group, an unsubstituted aliphatic group, a substituted aliphatic group, an unsubstituted aryl group, a substituted aryl group, an unsubstituted aryl-aliphatic group, a substituted aryl-aliphatic group, an ether group, an unsubstituted aliphatic ether group, a substituted aliphatic ether group;
(g) wherein R₂′ and R₂₅ are independently absent, a hydrogen, an unsubstituted aliphatic group, a substituted aliphatic group, an unsubstituted aryl group, a substituted aryl group, an unsubstituted aryl-aliphatic group, a substituted aryl-aliphatic group, an unsubstituted aliphatic ether group, or a substituted aliphatic ether group;
(h) wherein R₃, R₃′, and R₃″″ is absent, a hydrogen, an unsubstituted aliphatic group, a substituted aliphatic group, an unsubstituted amide group, a substituted amide group, an unsubstituted aliphatic ester group, a substituted aliphatic ester group, an unsubstituted aliphatic ether group, a substituted aliphatic ether group, an unsubstituted heterocyclic group, a substituted heterocyclic group, an unsubstituted heterocyclic-aliphatic group, or a substituted heterocyclic-aliphatic group;
(i) wherein R₈′ and R₂₇ are independently absent, a hydrogen, a halogen, a nitrile group, an ester group, an unsubstituted aliphatic ester group, a substituted aliphatic ester group, an unsubstituted amino group, a substituted amino group, an unsubstituted aliphatic amino group, a substituted aliphatic amino group, an amide group, a substituted aliphatic-amide group, an unsubstituted aliphatic-amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted aliphatic ether group, a substituted aliphatic ether group, un unsubstituted heterocyclic group, a substituted heterocyclic group, an unsubstituted heterocyclic-aliphatic group, or a substituted heterocyclic-aliphatic group;
(j) wherein R₄, R₅, R₆, and R₇ are independently absent, a hydrogen, a halogen, a nitrile group, an oxo group, an ester group, an unsubstituted aliphatic ester group, a substituted aliphatic ester group, an unsubstituted amino group, a substituted amino group, an unsubstituted aliphatic-amino group, a substituted aliphatic-amino group, an amide group, an unsubstituted aliphatic-amide group, a substituted aliphatic-amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted aliphatic ether group, a substituted aliphatic ether group, un unsubstituted heterocyclic group, a substituted heterocyclic group, an unsubstituted heterocyclic-aliphatic group, or a substituted heterocyclic-aliphatic group;
(k) wherein the substituents are as defined in the base paragraph(s).
   15. The compound of paragraph 14, wherein R₁′ or R₂′ of Formula XI and R₂₅ or R₂₆ of Formula XI′ is a substituted an unsubstituted aliphatic ester group, a substituted aliphatic ester group, an unsubstituted aliphatic ether group, or a substituted aliphatic ether group, preferably an unsubstituted or substituted aliphatic ether, such as an unsubstituted C₁-C₁₄ aliphatic ether or a substituted C₁-C₁₄ aliphatic ether, or an unsubstituted C₁-C₆ aliphatic ether or a substituted C₁-C₆ aliphatic ether.
   16. The compound of paragraph 14 or 15, wherein R₄ and R₅ are independently absent, hydrogen, an oxo group, an unsubstituted amino group, a substituted amino group, an unsubstituted aliphatic-amino group, or a substituted aliphatic-amino group.
   17. The compound of any one of paragraphs 13-16, wherein when J′ is OR₃, R₃ is a hydrogen, a substituted or unsubstituted aliphatic group, or a substituted or unsubstituted amino group.
   18. The compound of paragraph 14, wherein the compound has the structure of

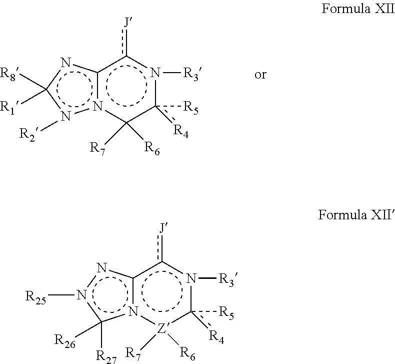

19. The compound of paragraph 18, wherein the compound has the structure of

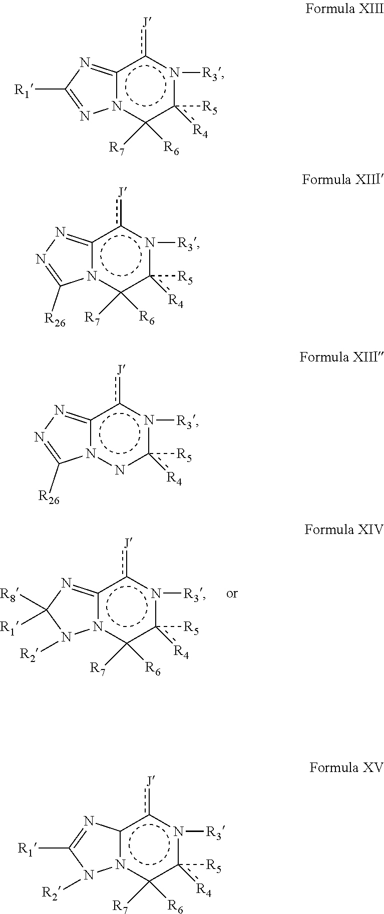

20. The compound of any one of paragraphs 1-6, wherein the compound has the structure of:

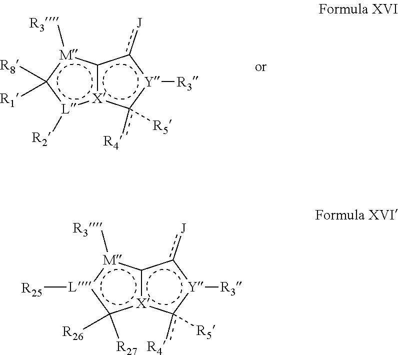

(a) wherein X′ is a carbon or a nitrogen;
(b) wherein L″, M″, and L″″ are independently a carbon or a nitrogen;
(c) wherein Y″ is a nitrogen;
(d) wherein J″ is an oxygen or OR₃;
(e) wherein R₁′ and R₂₆ are independently a hydrogen, a halogen, a nitrile group, a nitro group, an ester group, an unsubstituted aliphatic ester group, a substituted aliphatic ester group, an unsubstituted amino group, a substituted amino group, an unsubstituted aliphatic amino group, a substituted aliphatic amino group, an amide group, an unsubstituted aliphatic amide group, a substituted aliphatic amide group, an azide group, an unsubstituted aliphatic group, a substituted aliphatic group, an unsubstituted aryl group, a substituted aryl group, an unsubstituted aryl-aliphatic group, a substituted aryl-aliphatic group, an ether group, an unsubstituted aliphatic ether group, a substituted aliphatic ether group;
(f) wherein R₂′ and R₂₅ are independently absent, a hydrogen, an unsubstituted aliphatic group, a substituted aliphatic group, an unsubstituted aryl group, a substituted aryl group, an unsubstituted aryl-aliphatic group, a substituted aryl-aliphatic group, an unsubstituted aliphatic ether group, or a substituted aliphatic ether group;
(g) wherein R₃, R₃″, and R₃″″ is absent, a hydrogen, an unsubstituted aliphatic group, a substituted aliphatic group, an unsubstituted amide group, a substituted amide group, an unsubstituted aliphatic ester group, a substituted aliphatic ester group, an unsubstituted aliphatic ether group, a substituted aliphatic ether group, an unsubstituted heterocyclic group, a substituted heterocyclic group, an unsubstituted heterocyclic-aliphatic group, or a substituted heterocyclic-aliphatic group;
(h) wherein R₈′ and R₂₇ are independently absent, a hydrogen, a halogen, a nitrile group, an ester group, an unsubstituted aliphatic ester group, a substituted aliphatic ester group, an unsubstituted amino group, a substituted amino group, an unsubstituted aliphatic amino group, a substituted aliphatic amino group, an amide group, a substituted aliphatic-amide group, an unsubstituted aliphatic-amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted aliphatic ether group, a substituted aliphatic ether group, un unsubstituted heterocyclic group, a substituted heterocyclic group, an unsubstituted heterocyclic-aliphatic group, or a substituted heterocyclic-aliphatic group;
(i) wherein R₄ and R₅ are independently absent, a hydrogen, a halogen, a nitrile group, an oxo group, an ester group, an unsubstituted aliphatic ester group, a substituted aliphatic ester group, an unsubstituted amino group, a substituted amino group, an unsubstituted aliphatic-amino group, a substituted aliphatic-amino group, an amide group, an unsubstituted aliphatic-amide group, a substituted aliphatic-amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted aliphatic ether group, a substituted aliphatic ether group, un unsubstituted heterocyclic group, a substituted heterocyclic group, an unsubstituted heterocyclic-aliphatic group, or a substituted heterocyclic-aliphatic group;
(j) wherein the substituents are as defined in the base paragraph(s).
   21. The compound of paragraph 20, wherein R₁′ or R₂′ of Formula XVI and R₂₅ or R₂₆ of Formula XVI′ is a substituted an unsubstituted aliphatic ester group, a substituted aliphatic ester group, an unsubstituted aliphatic ether group, or a substituted aliphatic ether group, preferably an unsubstituted or substituted aliphatic ether, such as an unsubstituted C₁-C₁₄ aliphatic ether or a substituted C₁-C₁₄ aliphatic ether, or an unsubstituted C₁-C₆ aliphatic ether or a substituted C₁-C₆ aliphatic ether.
   22. The compound of paragraph 20 or 21, wherein R₄ and R₅ are independently absent, hydrogen, an oxo group, an unsubstituted amino group, a substituted amino group, an unsubstituted aliphatic-amino group, or a substituted aliphatic-amino group.
   23. The compound of any one of paragraphs 20-22, wherein when J″ is OR₃, R₃ is a hydrogen, a substituted or unsubstituted aliphatic group, or a substituted or unsubstituted amino group.
   24. The compound of paragraph 20, wherein the compound has the structure of

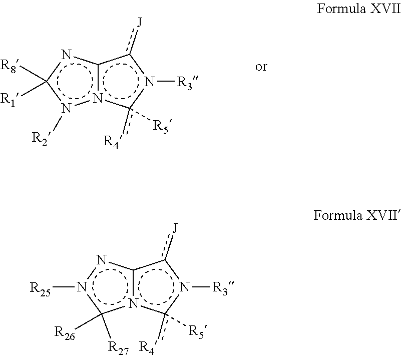

25. The compound of paragraph 24, wherein the compound has the structure of

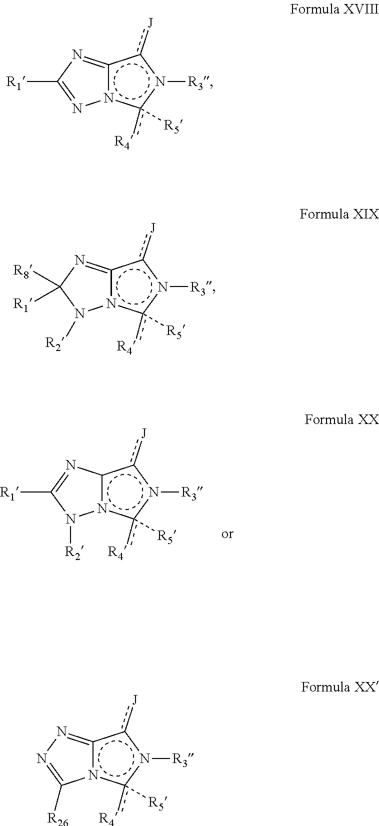

26. The compound of any one of paragraphs 7-25, wherein R₁, R₂, R₈, R₂₂, R₂₃, R₂₄, R₂₂′, R₂₂″, R₂₂′″, R₁′, R₂′, R₈′, R₂₅, R₂₆, and/or R₂₇ (such as R₁, R₂, and/or R₈ of Formula VI; R₂₂, R₂₃, and/or R₂₄ of Formula VI′; R₁, R₈, R₂₂′, R₂₃, and/or R₂₄ of Formula VI″; R₂₂″, R₂₂″, R₂₃, and/or R₂₄ of Formula VI′″; R₁′, R₂′, and/or R₈′ of Formula XI; R₂₅, R₂₆, and/or R₂₇ of Formula XI′; R₁′, R₂′, and/or R₈′ of Formula XVI; and/or R₂₅, R₂₆, and/or R₂₇ of Formula XVI′) are(is) an unsubstituted aliphatic ether or a substituted aliphatic ether, optionally an unsubstituted C₁-C₁₄ aliphatic ether or a substituted C₁-C₁₄ aliphatic ether, optionally an unsubstituted C₁-C₆ aliphatic ether or a substituted C₁-C₆ aliphatic ether.

27. The compound of paragraph 26, wherein R₁, R₂, R₈, R₂₂, R₂₃, R₂₄, R₂₂′, R₂₂″, R₂₂′″, R₁′, R₂′, R₈′, R₂₅, R₂₆, and/or R₂₇ are(is) a substituted C₁-C₆ aliphatic ether having the structure of

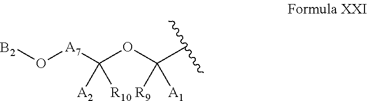

(a) wherein A₁ and A₂ are independently a hydrogen, a halogen, a hydroxyl group, a thiol group, a cyanide group, a nitrile group, an unsubstituted aliphatic group, a substituted aliphatic group, an unsubstituted heterocyclic group, a substituted heterocyclic group, an unsubstituted aryl group, or a substituted aryl group;
(b) wherein A₇ is a methylene group or an ethylene group;
(c) wherein R₉ and R₁₀ are independently a hydrogen, a hydroxyl group, an unsubstituted alkyl group, or a substituted alkyl group, optionally wherein R₉ and R₁₀ are independently a substituted alkyl group that together form a ring; and
(d) wherein B₂ is hydrogen or a phosphoramidate, wherein the substituents are as defined in the base paragraph(s).
   28. The compound of paragraph 27, wherein the substituted C₁-C₆ aliphatic ether has the structure of

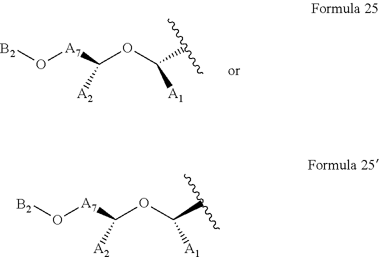

29. The compound of paragraph 27, wherein the substituted C₁-C₆ aliphatic ether has the structure of

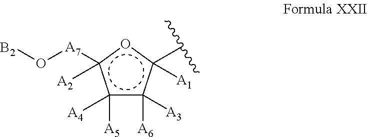

wherein A₃, A₄, A₅, and A₆ are independently absent, a hydrogen, a halogen, a hydroxyl group, a thiol group, an unsubstituted aliphatic group, a substituted aliphatic group, an unsubstituted heterocyclic group, a substituted heterocyclic group, an unsubstituted aryl group, or a substituted aryl group
   30. The compound of paragraph 29, wherein the substituted C₁-C₆ aliphatic ether has the structure of

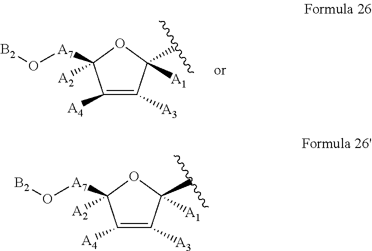

(a) wherein A₁ is an unsubstituted alkyl group, a substituted alkyl group, a cyanide group, a nitrile group, a hydroxyl group, or a halogen; and
(b) wherein A₂ is a hydrogen, a halogen, a hydroxyl group, a thiol group, an unsubstituted aliphatic group, a substituted aliphatic group, an unsubstituted heterocyclic group, a substituted heterocyclic group, an unsubstituted aryl group, or a substituted aryl group.
   31. The compound of paragraph 29, wherein the substituted C₁-C₆ aliphatic ether has the structure of

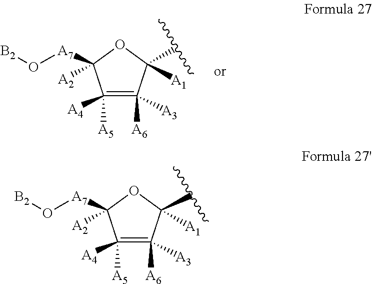

(a) wherein A₁ is an unsubstituted alkyl group, a substituted alkyl group, a cyanide group, a nitrile group, a hydroxyl group, or a halogen; and
(b) wherein A₂ is a hydrogen, a halogen, a hydroxyl group, a thiol group, an unsubstituted aliphatic group, a substituted aliphatic group, an unsubstituted heterocyclic group, a substituted heterocyclic group, an unsubstituted aryl group, or a substituted aryl group;
(c) optionally wherein A₂, A₄, and A₆ are hydrogen, or A₁ is cyanide, or A₃ and/or A₆ are hydroxyl, or a combination thereof; and
(d) optionally wherein Formula 27′ is

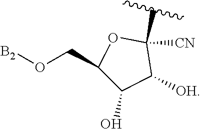

32. The compound of any one of paragraphs 27-31, wherein B₂ is a phosphoramidate having the structure of

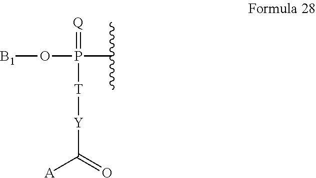

(a) wherein B₁ is a hydrogen, an unsubstituted aliphatic group, a substituted aliphatic group, an unsubstituted aryl group, or a substituted aryl group;
(b) wherein Q is an oxygen or a sulfur;
(c) wherein T is an oxygen, a sulfur, NR₁₁, CR₁₁R₁₂, CR₁₁W₁, or CW₁W₂, wherein R₁₁ and R₁₂ are independently a hydrogen, an unsubstituted alkyl group, a substituted alkyl group, an unsubstituted aryl group, a substituted aryl group, and wherein W₁ and W₂ are independently a heteroatom;
(d) wherein Y is an unsubstituted aliphatic group, a substituted aliphatic group, an unsubstituted heterocyclic group, a substituted heterocyclic group, an unsubstituted aryl group, or a substituted aryl group; and
(e) wherein A is an unsubstituted aliphatic group, a substituted aliphatic group, an unsubstituted aryl group, a substituted aryl group, an alkoxyl, —CM₁alkyl, —CM₁aryl, or —CM₁heterocyclic, wherein M₁ is an oxygen or a methylene group, optionally wherein A is —O-alkyl, wherein the alkyl can be a substituted or unsubstituted alkyl, such as an unsubstituted liner, branched, or cyclic C₁-C₆ alkyl, an unsubstituted liner, branched, or cyclic C₁-C₄ alkyl, or an unsubstituted liner, branched, or cyclic C₁-C₃ alkyl.
   33. The compound of any one of paragraphs 1-6, wherein the compound has the structure of

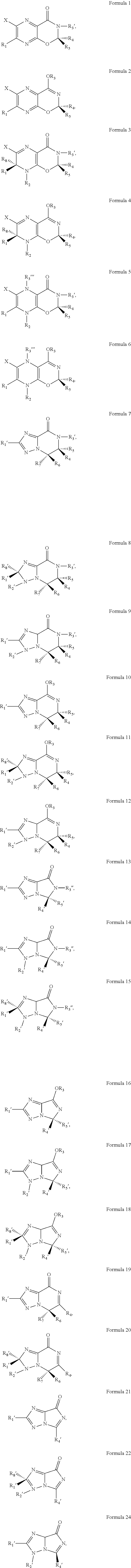

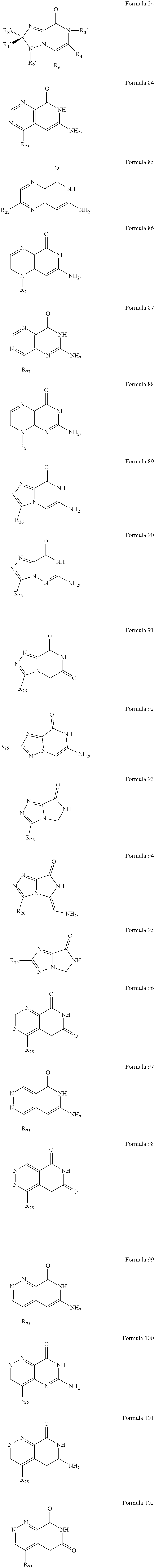

a tautomer thereof,
(a) wherein X is a hydrogen, a halogen, a nitrile group, a nitro group, an ester group, an unsubstituted aliphatic ester group, a substituted aliphatic ester group, an unsubstituted amino group, a substituted amino group, an amide group, an unsubstituted aliphatic-amide group, a substituted aliphatic-amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted heterocyclic group, a substituted heterocyclic group, an unsubstituted heterocyclic-aliphatic group, or a substituted heterocyclic-aliphatic group;
(b) wherein R₁, R₁′, R₂, R₂′, R₂₂, R₂₃, R₂₅, and R₂₆ are independently a hydrogen, a halogen, a nitrile group, a nitro group, an ester group, an unsubstituted aliphatic ester group, a substituted aliphatic ester group, an unsubstituted amino group, a substituted amino group, an unsubstituted aliphatic amino group, a substituted aliphatic amino group, an amide group, an unsubstituted aliphatic amide group, a substituted aliphatic amide group, an azide group, an unsubstituted aliphatic group, a substituted aliphatic group, an unsubstituted aryl group, a substituted aryl group, an unsubstituted aryl-aliphatic group, a substituted aryl-aliphatic group, an ether group, an unsubstituted aliphatic ether group, a substituted aliphatic ether group;
(c) wherein R₃, R₃′, R₃″, and R₃′″ are independently absent, a hydrogen, an unsubstituted aliphatic group, a substituted aliphatic group, an unsubstituted aliphatic ester group, a substituted aliphatic ester group, an unsubstituted aliphatic ether group, a substituted aliphatic ether group, an unsubstituted heterocyclic group, a substituted heterocyclic group, an unsubstituted heterocyclic-aliphatic group, or a substituted heterocyclic-aliphatic group;
(d) wherein R₈ and R₈′ are independently absent, a hydrogen, a halogen, a nitrile group, an ester group, an unsubstituted aliphatic ester group, a substituted aliphatic ester group, an unsubstituted amino group, a substituted amino group, an unsubstituted aliphatic amino group, a substituted aliphatic amino group, an amide group, a substituted aliphatic-amide group, an unsubstituted aliphatic-amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted aliphatic ether group, a substituted aliphatic ether group, un unsubstituted heterocyclic group, a substituted heterocyclic group, an unsubstituted heterocyclic-aliphatic group, or a substituted heterocyclic-aliphatic group; and
(e) wherein R₄, R₄′, R₅, R₅′, R₆, and R₇ are independently absent, a hydrogen, a halogen, a nitrile group, an oxo group, an ester group, an unsubstituted aliphatic ester group, a substituted aliphatic ester group, an unsubstituted amino group, a substituted amino group, an unsubstituted aliphatic-amino group, a substituted aliphatic-amino group, an amide group, an unsubstituted aliphatic-amide group, a substituted aliphatic-amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted aliphatic ether group, a substituted aliphatic ether group, un unsubstituted heterocyclic group, a substituted heterocyclic group, an unsubstituted heterocyclic-aliphatic group, or a substituted heterocyclic-aliphatic group;
(f) wherein the substituents are as defined in the base paragraph(s). 34. The compound of paragraph 33, wherein R₁ of Formulae 1-6; R₁′ of Formulae 7-24; R₂ of Formulae 86 and 88; R₂₂ of Formula 85; R₂₃ of Formulae 84, 87, and 96-102; R₂₅ of Formulae 92 and 95; and/or R₂₆ of Formulae 89-91, 93, and 94 are(is) an unsubstituted aliphatic ether or a substituted aliphatic ether, optionally an unsubstituted C₁-C₁₄ aliphatic ether or a substituted C₁-C₁₄ aliphatic ether, optionally an unsubstituted C₁-C₆ aliphatic ether or a substituted C₁-C₆ aliphatic ether.
   35. The compound of paragraph 34, wherein R₁ of Formulae 1-6; R₁′ of Formulae 7-24; R₂ of Formulae 86 and 88; R₂₂ of Formula 85; R₂₃ of Formulae 84, 87, and 96-102; R₂₅ of Formulae 92 and 95; and/or R₂₆ of Formulae 89-91, 93, and 94 are(is) a substituted C₁-C₆ aliphatic ether having the structure of

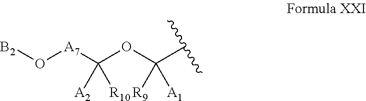

(a) wherein A₁ and A₂ are independently a hydrogen, a halogen, a hydroxyl group, a thiol group, a cyanide group, a nitrile group, an unsubstituted aliphatic group, a substituted aliphatic group, an unsubstituted heterocyclic group, a substituted heterocyclic group, an unsubstituted aryl group, or a substituted aryl group;
(b) wherein A₇ is a methylene group or an ethylene group;
(c) wherein R₉ and R₁₀ are independently a hydrogen, a hydroxyl group, an unsubstituted alkyl group, or a substituted alkyl group, optionally wherein R₉ and R₁₀ are independently a substituted alkyl group that together form a ring; and
(d) wherein B₂ is hydrogen or a phosphoramidate,
wherein the substituents are as defined in the base paragraph(s).
   36. The compound of paragraph 35, wherein the substituted C₁-C₆ aliphatic ether has the structure of

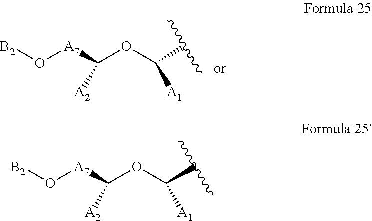

37. The compound of paragraph 35, wherein the substituted C₁-C₆ aliphatic ether has the structure of

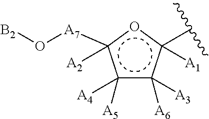

wherein A₃, A₄, A₅, and A₆ are independently absent, a hydrogen, a halogen, a hydroxyl group, a thiol group, an unsubstituted aliphatic group, a substituted aliphatic group, an unsubstituted heterocyclic group, a substituted heterocyclic group, an unsubstituted aryl group, or a substituted aryl group
   38. The compound of paragraph 37, wherein the substituted C₁-C₆ aliphatic ether has the structure of

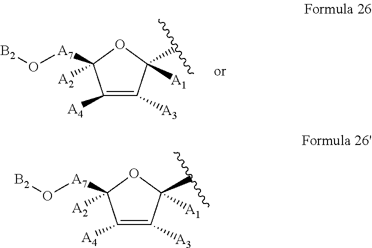

(a) wherein A₁ is an unsubstituted alkyl group, a substituted alkyl group, a cyanide group, a nitrile group, a hydroxyl group, or a halogen; and
(b) wherein A₂ is a hydrogen, a halogen, a hydroxyl group, a thiol group, an unsubstituted aliphatic group, a substituted aliphatic group, an unsubstituted heterocyclic group, a substituted heterocyclic group, an unsubstituted aryl group, or a substituted aryl group.
   39. The compound of paragraph 37, wherein the substituted C₁-C₆ aliphatic ether has the structure of

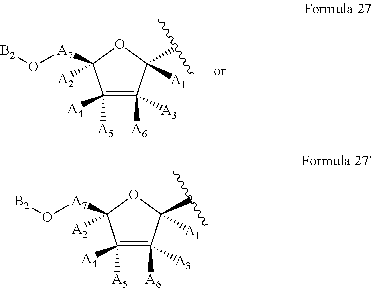

(a) wherein A₁ is an unsubstituted alkyl group, a substituted alkyl group, a cyanide group, a nitrile group, a hydroxyl group, or a halogen; and
(b) wherein A₂ is a hydrogen, a halogen, a hydroxyl group, a thiol group, an unsubstituted aliphatic group, a substituted aliphatic group, an unsubstituted heterocyclic group, a substituted heterocyclic group, an unsubstituted aryl group, or a substituted aryl group;
(c) optionally wherein A₂, A₄, and A₆ are hydrogen, or A₁ is cyanide, or A₃ and/or A₆ are hydroxyl, or a combination thereof; and

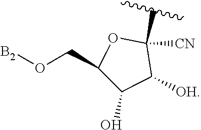

(d) optionally wherein Formula 27′ is OH
   40. The compound of any one of paragraphs 35-39, wherein B₂ is a phosphoramidate having the structure of

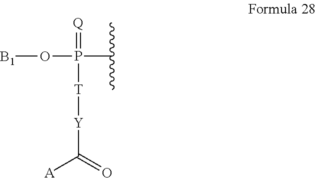

(a) wherein B₁ is a hydrogen, an unsubstituted aliphatic group, a substituted aliphatic group, an unsubstituted aryl group, or a substituted aryl group;
(b) wherein Q is an oxygen or a sulfur;
(c) wherein T is an oxygen, a sulfur, NR₁₁, CR₁₁R₁₂, CR₁₁W₁, or CW₁W₂, wherein R₁₁ and R₁₂ are independently a hydrogen, an unsubstituted alkyl group, a substituted alkyl group, an unsubstituted aryl group, a substituted aryl group, and wherein W₁ and W₂ are independently a heteroatom;
(d) wherein Y is an unsubstituted aliphatic group, a substituted aliphatic group, an unsubstituted heterocyclic group, a substituted heterocyclic group, an unsubstituted aryl group, or a substituted aryl group; and
(e) wherein A is an unsubstituted aliphatic group, a substituted aliphatic group, an unsubstituted aryl group, a substituted aryl group, an alkoxyl, —CM₁alkyl, —CM₁aryl, or —CM₁heterocyclic, wherein M₁ is an oxygen or a methylene group, optionally wherein A is —O-alkyl, where the alkyl can be a substituted or unsubstituted alkyl, such as an unsubstituted liner, branched, or cyclic C₁-C₆ alkyl, an unsubstituted liner, branched, or cyclic C₁-C₄ alkyl, or an unsubstituted liner, branched, or cyclic C₁-C₃ alkyl.
   41. The compound of any one of paragraphs 34-40, wherein X is a hydrogen, an unsubstituted aliphatic group, a substituted aliphatic group, an unsubstituted aryl group, a substituted aryl group, an unsubstituted heterocyclic group, or a substituted heterocyclic group;
wherein R₃, R₃′, R₃″, R₃′″, R₈, and R₈′ are independently absent, a hydrogen, an unsubstituted aliphatic group, a substituted aliphatic group, an unsubstituted heterocyclic group, or a substituted heterocyclic group; and/or
wherein R₄, R₄′, R₅, R₅′, R₆, and R₇ are independently absent, a hydrogen, an oxo group, an unsubstituted amino group, a substituted amino group, an unsubstituted aliphatic-amino group, a substituted aliphatic-amino group, an amide group, an unsubstituted aliphatic-amide group, a substituted aliphatic-amide group, an unsubstituted aliphatic group, a substituted aliphatic group, an unsubstituted aryl group, a substituted aryl group, an unsubstituted aryl-aliphatic group, a substituted aryl-aliphatic group, un unsubstituted heterocyclic group, a substituted heterocyclic group, an unsubstituted heterocyclic-aliphatic group, or a substituted heterocyclic-aliphatic group.
   42. A pharmaceutical composition or dosage unit comprising
the compound of any one of paragraphs 1-41; and
a pharmaceutically acceptable excipient,
wherein the compound is in an effective amount to prevent, treat, or ameliorate one or more symptoms associated with a viral infection in a subject.
   43. The pharmaceutical composition or dosage unit of paragraph 42 further comprising a second active agent, optionally more than one second active agent.
   44. The pharmaceutical composition or dosage unit of paragraph 43, wherein the second active agent is an antiviral agent or an anti-inflammatory agent.
   45. A method for preventing, treating, or ameliorating one or more symptoms associated with a viral infection in a subject comprising
(i) administering to the subject an effective amount of the compound of paragraphs 1-41 to prevent, treat, or ameliorate one or more symptoms associated with the viral infection in the subject.
   46. The method of paragraph 45, wherein the subject is a mammal.
   47. The method of paragraph 45 or paragraph 46, wherein the compound is administered by oral administration, parenteral administration, inhalation, mucosal administration, topical or a combination thereof.
   48. The method of any one of paragraphs 45-47 further comprising administering to the subject a second active agent, optionally more than one active agent, prior to, during, and/or subsequent to step (i).
   49. The method of paragraph 48, wherein the second active agent is an antiviral agent or an anti-inflammatory agent.
   50. The method of any one of paragraphs 45-49, wherein the viral infection is caused by double stranded DNA viruses, single stranded DNA viruses, double stranded RNA viruses, single stranded positive sense RNA viruses, single stranded negative sense RNA viruses, positive sense single stranded RNA viruses that replicate through a DNA intermediate, double stranded DNA viruses that replicate through a single stranded RNA intermediate, single stranded RNA viruses with reverse transcriptase, or double stranded DNA viruses with reverse transcriptase, or a combination thereof.
   51. The method of any one of paragraphs 45-50, wherein one or more functional groups of the compound form one or more hydrogen bonds with a first nucleoside in a RNA or a DNA of the subject, optionally at least two hydrogen bonds with the first nucleoside in the RNA or the DNA of the subject, and wherein the compound is recognized as a second nucleoside or nucleotide in the RNA or the DNA of the subject.

The present invention will be further understood by reference to the following non-limiting examples.

### EXAMPLES

### Example 1. Formula 29

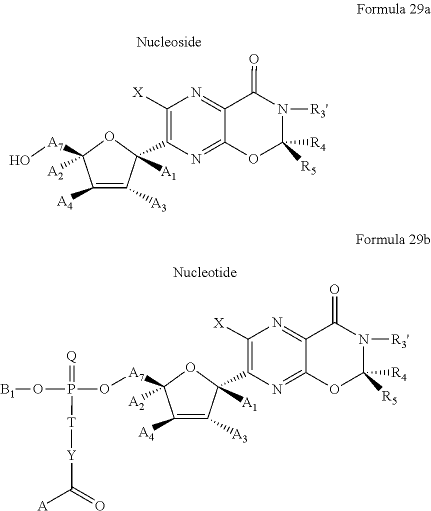

X is a hydrogen, a halogen, a nitrile group, a nitro group, an ester group, an unsubstituted aliphatic ester group, a substituted aliphatic ester group, an unsubstituted amino group, a substituted amino group, an amide group, an aliphatic-amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted heterocyclic group, a substituted heterocyclic group, an unsubstituted heterocyclic-aliphatic group, or a substituted heterocyclic-aliphatic group;
R₃′ is a hydrogen, an unsubstituted aliphatic group, a substituted aliphatic group, an unsubstituted aliphatic ester group, a substituted aliphatic ester group, an unsubstituted aliphatic ether group, a substituted aliphatic ether group, an unsubstituted heterocyclic group, a substituted heterocyclic group, an unsubstituted heterocyclic-aliphatic group, or a substituted heterocyclic-aliphatic group;
R₄ and R₅ are independently a hydrogen, an oxygen, a halogen, a nitrile group, an ester group, an unsubstituted aliphatic ester group, a substituted aliphatic ester group, an unsubstituted amino group, a substituted amino group, an amide group, an aliphatic-amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted aliphatic ether group, a substituted aliphatic ether group, un unsubstituted heterocyclic group, a substituted heterocyclic group, an unsubstituted heterocyclic-aliphatic group, or a substituted heterocyclic-aliphatic group;
A1 is an unsubstituted alkyl group, a substituted alkyl group, a cyanide group, a nitrile group, a hydroxyl group, or a halogen;
A₂, A₃, and A₄ are independently a hydrogen, a halogen, a hydroxyl group, a thiol group, an unsubstituted aliphatic group, a substituted aliphatic group, an unsubstituted heterocyclic group, a substituted heterocyclic group, an unsubstituted aryl group, or a substituted aryl group;
A₇ is a methylene group or an ethylene group;
B₁ is a hydrogen, an unsubstituted aliphatic group, a substituted aliphatic group, an unsubstituted aryl group, or a substituted aryl group;
Q is an oxygen or a sulfur;
T is an oxygen, a sulfur, NR₁₁, CR₁₁R₁₂, CR₁₁W₁, or CW₁W₂, wherein R₁₁ and R₁₂ are independently a hydrogen, an unsubstituted alkyl group, a substituted alkyl group, an unsubstituted aryl group, a substituted aryl group, and wherein W₁ and W₂ are independently a heteroatom;
Y is an unsubstituted aliphatic group, a substituted aliphatic group, an unsubstituted heterocyclic group, a substituted heterocyclic group, an unsubstituted aryl group, or a substituted aryl group; and
A is an unsubstituted aliphatic group, a substituted aliphatic group, an unsubstituted aryl group, a substituted aryl group, an alkoxyl, —CM₁alkyl, —CM₁aryl, or —CM₁heterocyclic, wherein M₁ is an oxygen or a methylene group, optionally wherein A is —O-alkyl, where the alkyl can be a substituted or unsubstituted alkyl, such as an unsubstituted liner, branched, or cyclic C₁-C₆ alkyl, an unsubstituted liner, branched, or cyclic C₁-C₄ alkyl, or an unsubstituted liner, branched, or cyclic C₁-C₃ alkyl;
the substituents are independently a phosphoramidate, a heterocyclic group, a halogen, a sulfonic acid, an azide group, a cyanate group, an isocyanate group, a nitrate group, a nitrile group, an isonitrile group, a nitrosooxy group, a nitroso group, a nitro group, an aldehyde group, an alkoxy group, an acyl halide group, a carboxylic acid group, a carboxylate group, an alkyl group, a heteroalkyl group, an alkenyl group, a heteroalkenyl group, an alkynyl group, a heteroalkynyl group, an aryl group, a heteroaryl group, polyaryl group, a heteropoly aryl group, a alkyl aryl group, an amino group, an ester group, a hydroxyl group, a thiol group, a sulfonyl group, an ester group, an aliphatic ester group, an amide group, an aliphatic amide group, an azo group, an acyl group, a carbonyl group, a carbonate ester group, an ether group, an aminooxy group, or a hydroxyamino group.

### Example 2. Formula 30

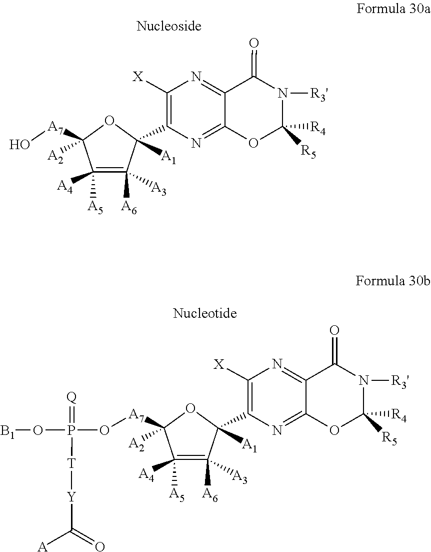

X, R₃′, R₄, R₅, A₁-A₄, A₇, Q, T, Y, B₁, and A are as described in Example 1; and
A₅ and A₆ are independently a hydrogen, a halogen, a hydroxyl group, a thiol group, an unsubstituted aliphatic group, a substituted aliphatic group, an unsubstituted heterocyclic group, a substituted heterocyclic group, an unsubstituted aryl group, or a substituted aryl group,
where the substituents are as described in Example 1.

### Example 3. Formula 31

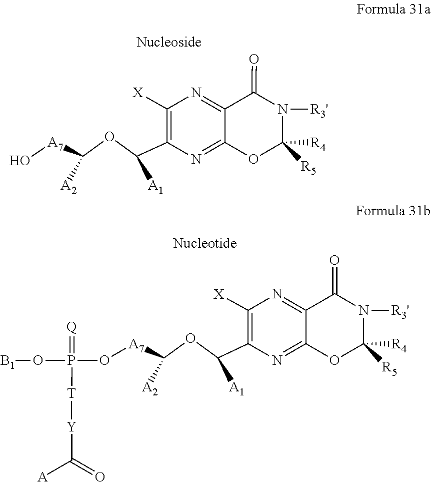 [0376] X, R₃′, R₄, R₅, A₁, A₂, A₇, Q, T, Y, B₁, and A are as described in Example 1.

### Example 4. Formula 32

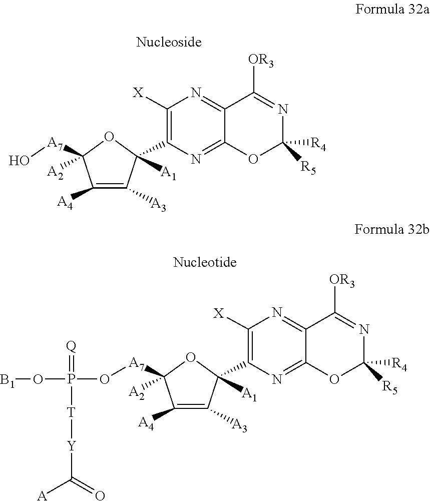

X, R₄, R₅, A₁-A₄, A₇, Q, T, Y, B₁, and A are as described in Example 1; and
R₃ is a hydrogen, an unsubstituted aliphatic group, a substituted aliphatic group, an unsubstituted aliphatic ester group, a substituted aliphatic ester group, an unsubstituted aliphatic ether group, a substituted aliphatic ether group, an unsubstituted heterocyclic group, a substituted heterocyclic group, an unsubstituted heterocyclic-aliphatic group, or a substituted heterocyclic-aliphatic group,
where the substituents are as described in Example 1.

### Example 5. Formula 33

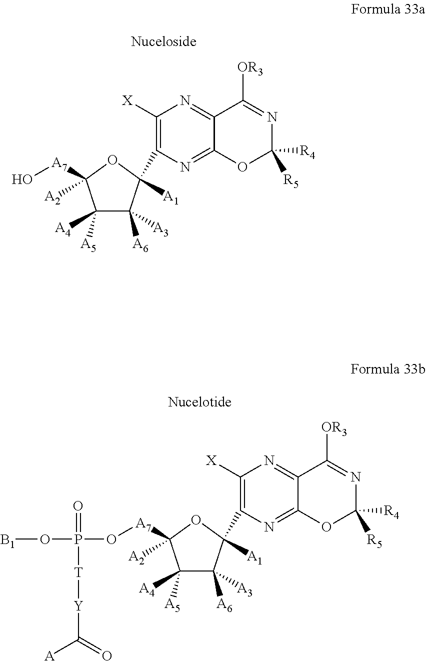 [0382] X, R₄, R₅, A1-A₄, A₇, Q, T, Y, B₁, and A are as described in Example 1;
A₅ and A₆ are as described in Example 2; and
R₃ is as described in Example 4.

### Example 6. Formula 36

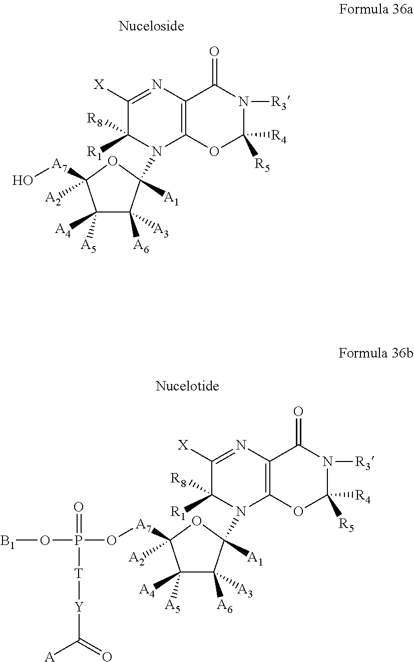 [0386] X, R₃, R₄, R₅, A₁, A₂, A₇, Q, T, Y, B₁, and A are as described in Example 1;
A₅ and A₆ are as described in Example 2;
R₁ is a hydrogen, a halogen, a nitrile group, a nitro group, an ester group, an unsubstituted aliphatic ester group, a substituted aliphatic ester group, .an unsubstituted amino group, a substituted amino group, an amide group, an unsubstituted amide group, a substituted amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted aliphatic ether group, a substituted aliphatic ether group; and
R₈ is a hydrogen, a halogen, a nitrile group, an ester group, an unsubstituted aliphatic ester group, a substituted aliphatic ester group, an unsubstituted amino group, a substituted amino group, an amide group, an aliphatic-amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted aliphatic ether group, a substituted aliphatic ether group, un unsubstituted heterocyclic group, a substituted heterocyclic group, an unsubstituted heterocyclic-aliphatic group, or a substituted heterocyclic-aliphatic group,
where the substituents are as described in Example 1.

### Example 7. Formula 47

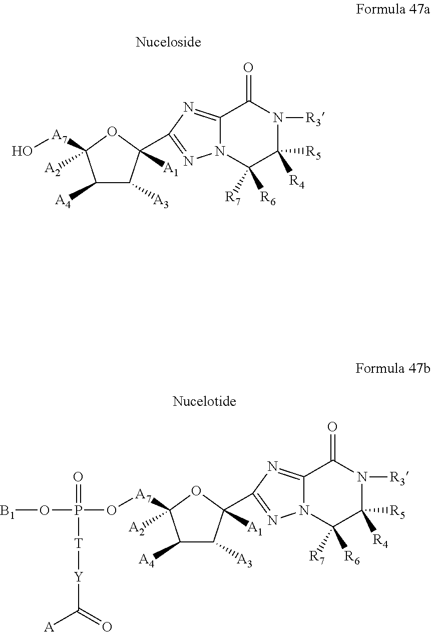

R₃′, R₄, R₅, A₁-A₄, A₇, Q, T, Y, B₁, and A are as described in Example 1; and
R₆ and R₇ are independently a hydrogen, an oxygen, a halogen, a nitrile group, an ester group, an unsubstituted aliphatic ester group, a substituted aliphatic ester group, an unsubstituted amino group, a substituted amino group, an amide group, an aliphatic-amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted aliphatic ether group, a substituted aliphatic ether group, un unsubstituted heterocyclic group, a substituted heterocyclic group, an unsubstituted heterocyclic-aliphatic group, or a substituted heterocyclic-aliphatic group,
where the substituents are as described in Example 1.

### Example 8. Formula 51

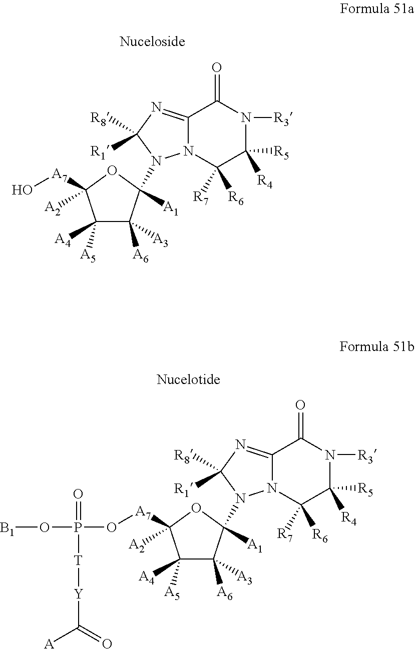 [0396] R₃′, R₄, R₅, A₁-A₄, A₇, Q, T, Y, B₁, and A are as described in Example 1;
R₆ and R₇ are as described in Example 7;
A₅ and A₆ are as described in Example 2;
R₁′ is a hydrogen, a halogen, a nitrile group, a nitro group, an ester group, an unsubstituted aliphatic ester group, a substituted aliphatic ester group, .an unsubstituted amino group, a substituted amino group, an amide group, an unsubstituted amide group, a substituted amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted aliphatic ether group, a substituted aliphatic ether group; and
R₈′ is a hydrogen, a halogen, a nitrile group, an ester group, an unsubstituted aliphatic ester group, a substituted aliphatic ester group, an unsubstituted amino group, a substituted amino group, an amide group, an aliphatic-amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted aliphatic ether group, a substituted aliphatic ether group, un unsubstituted heterocyclic group, a substituted heterocyclic group, an unsubstituted heterocyclic-aliphatic group, or a substituted heterocyclic-aliphatic group,
where the substituents are as described in Example 1.

### Example 9. Formula 54

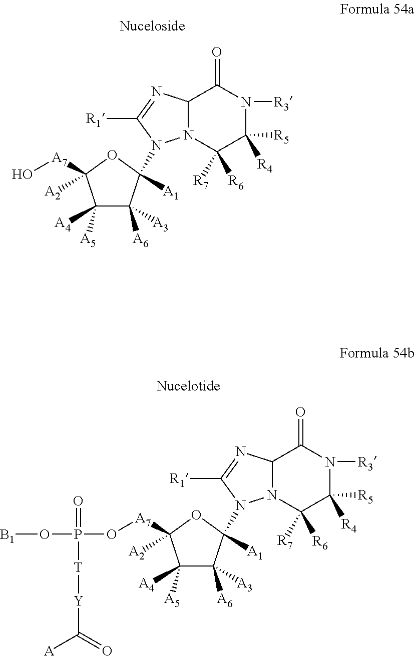 [0403] R₃′, R₄, R₅, A1-A₄, A₇, Q, T, Y, B₁, and A are as described in Example 1;
R₁′ is as described in Example 8;
R₆ and R₇ are as described in Example 7; and
A₅ and A₆ are as described in Example 2.

### Example 10. Formula 57

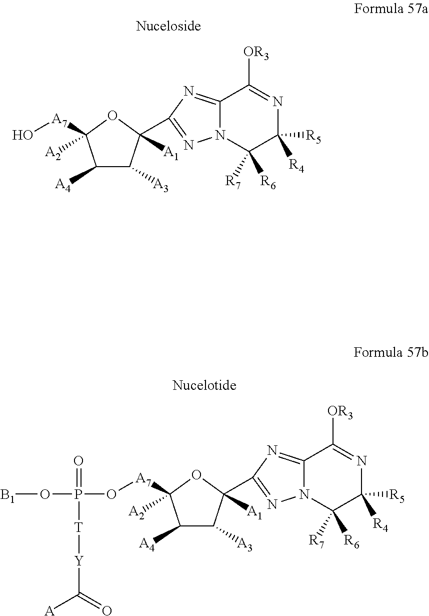 [0408] R₄, R₅, A₁-A₄, A₇, Q, T, Y, B₁, and A are as described in Example 1;
R₃ is as described in Example 4;
R₆ and R₇ are as described in Example 7; and
A₅ and A₆ are as described in Example 2.

### Example 11. Formula 63

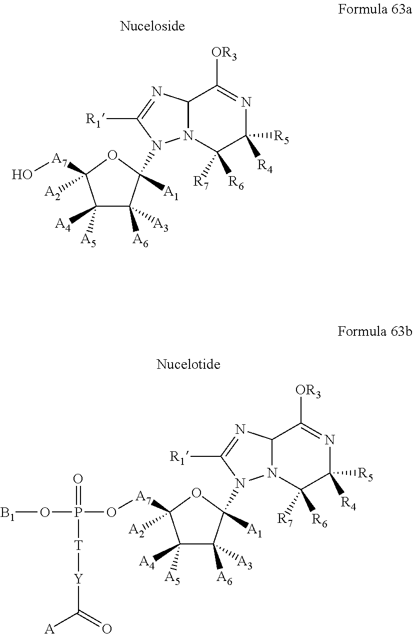 [0413] R₄, R₅, A₁-A₄, A₇, Q, T, Y, B₁, and A are as described in Example 1;
R₁′ is as described in Example 8;
R₃ is as described in Example 4;
R₆ and R₇ are as described in Example 7; and
A₅ and A₆ are as described in Example 2.

### Example 12. Formula 64

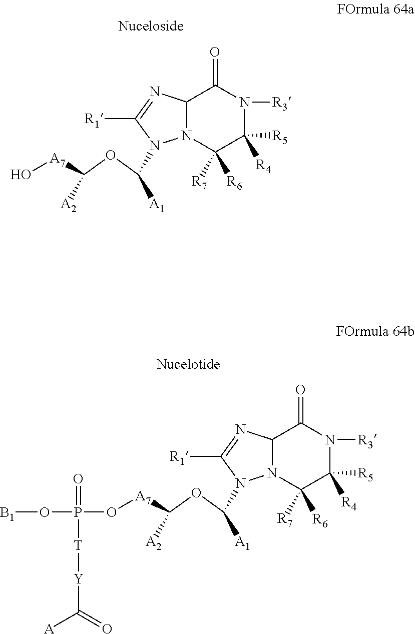 [0419] R₃′, R₄, R₅, A₁, A₂, A₇, Q, T, Y, B₁, and A are as described in Example 1;
R₁′ is as described in Example 8; and
R₆ and R₇ are as described in Example 7.

### Example 13. Formula 65

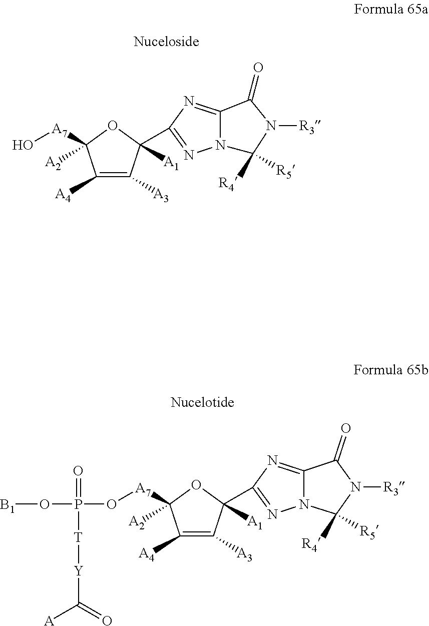 [0423] A₁-A₄, A₇, Q, T, Y, B₁, and A are as described in Example 1;
R₃″ is a hydrogen, an unsubstituted aliphatic group, a substituted aliphatic group, an unsubstituted aliphatic ester group, a substituted aliphatic ester group, an unsubstituted aliphatic ether group, a substituted aliphatic ether group, an unsubstituted heterocyclic group, a substituted heterocyclic group, an unsubstituted heterocyclic-aliphatic group, or a substituted heterocyclic-aliphatic group; and
R₄′ and R₅′ are independently a hydrogen, an oxygen, a halogen, a nitrile group, an ester group, an unsubstituted aliphatic ester group, a substituted aliphatic ester group, an unsubstituted amino group, a substituted amino group, an amide group, an aliphatic-amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted aliphatic ether group, a substituted aliphatic ether group, un unsubstituted heterocyclic group, a substituted heterocyclic group, an unsubstituted heterocyclic-aliphatic group, or a substituted heterocyclic-aliphatic group where the substituents are as described in Example 1.

### Example 14. Formula 69

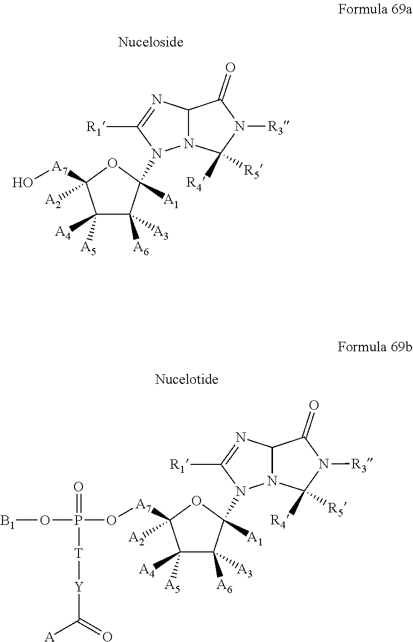 [0427] A₁-A₄, A₇, Q, T, Y, B₁, and A are as described in Example 1;
R₁′ is as described in Example 8;
A₅ and A₆ are as described in Example 2; and
R₃″, R₄′, and R₅′ are as described in Example 13.

### Example 15. Formula 71

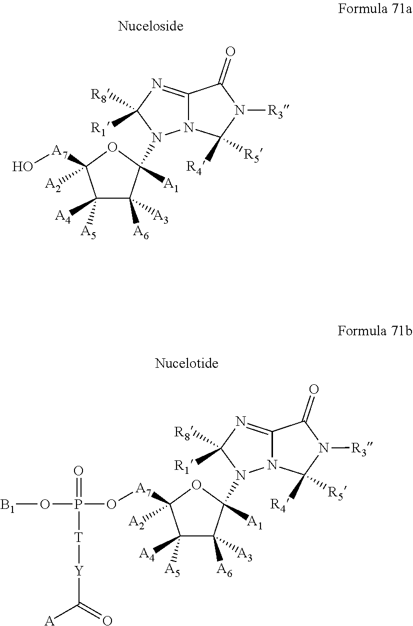 [0432] A₁-A₄, A₇, Q, T, Y, B₁, and A are as described in Example 1;
R₁′ and R₈′ are as described in Example 8; and
R₃″, R₄′, and R₅′ are as described in Example 13.

### Example 16. Formula 75

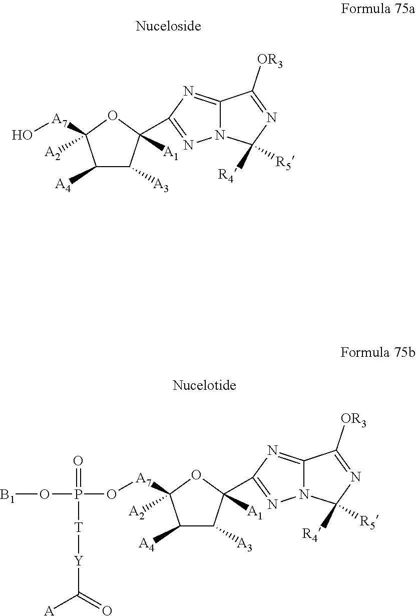 [0436] R₄′, R₅′, A₁-A₄, A₇, Q, T, Y, B₁, and A are as described in Example 1;
R₃ is as described in Example 4; and
As and A₆ are as described in Example 2.

### Example 17. Formula 78

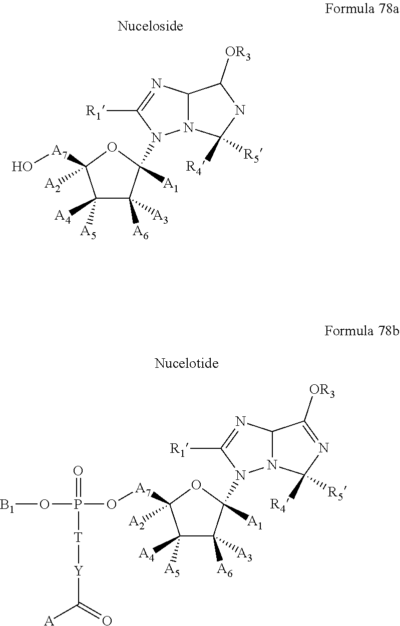 [0440] R₄′, R₅′, A₁-A₄, A₇, Q, T, Y, B₁, and A are as described in Example 1;
R₁′ is as described in Example 8;
R₃ is as described in Example 4; and
A₅ and A₆ are as described in Example 2.

### Example 18. Formula 80

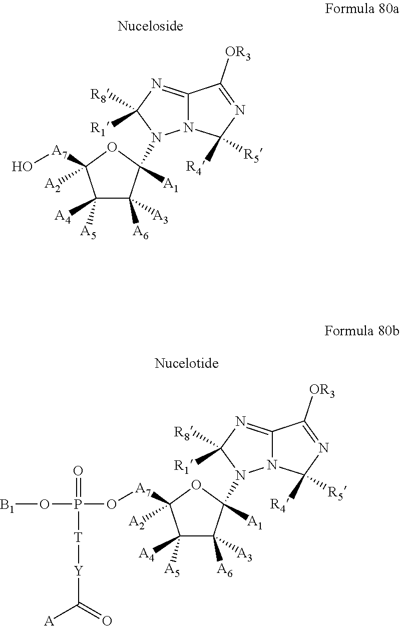 [0445] R₄′, R₅′, A₁-A₄, A₇, Q, T, Y, B₁, and A are as described in Example 1;
R₁′ and R₈′ are as described in Example 8; and
R₃ is as described in Example 4.

### Example 19. Additional Exemplary Compounds

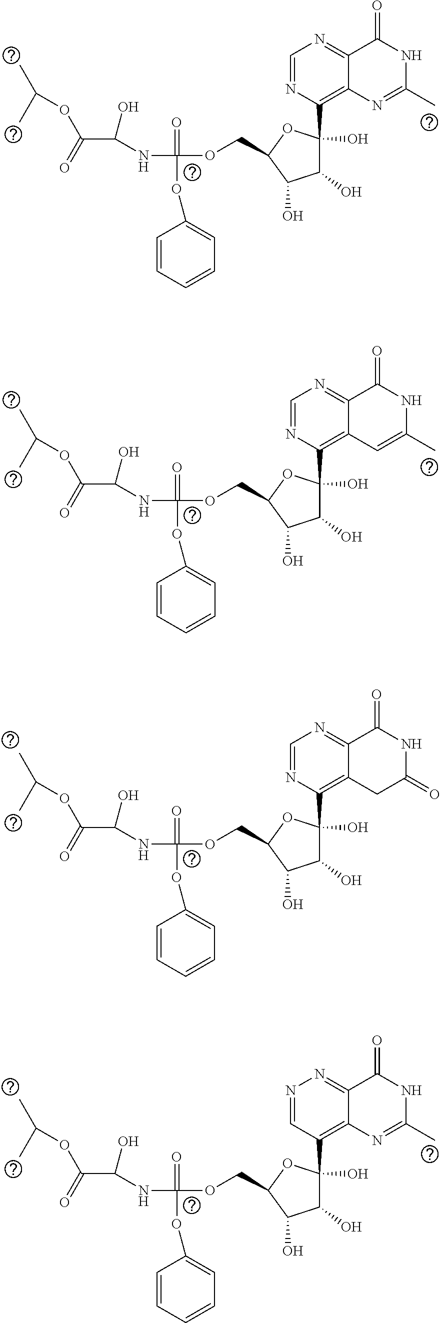

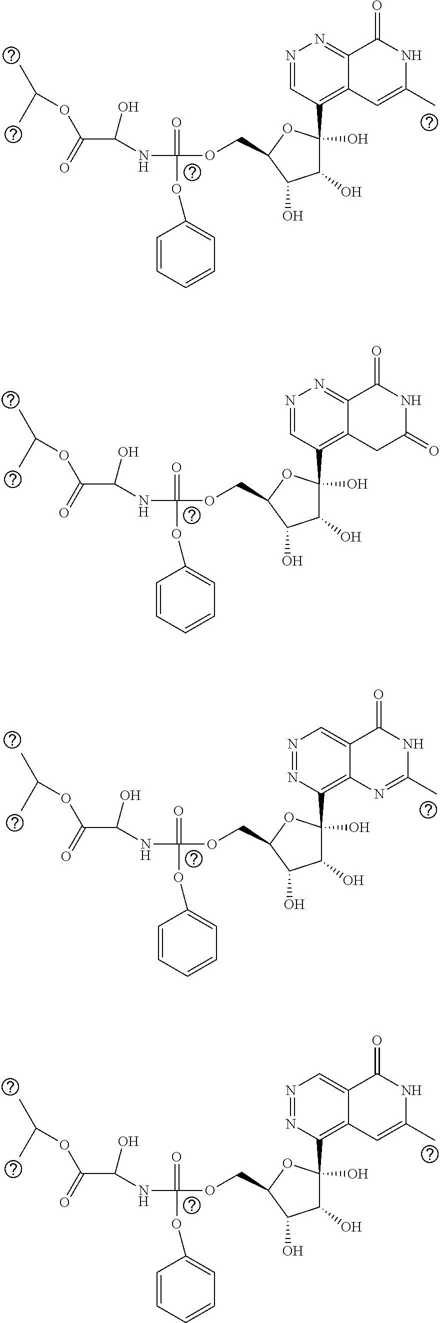

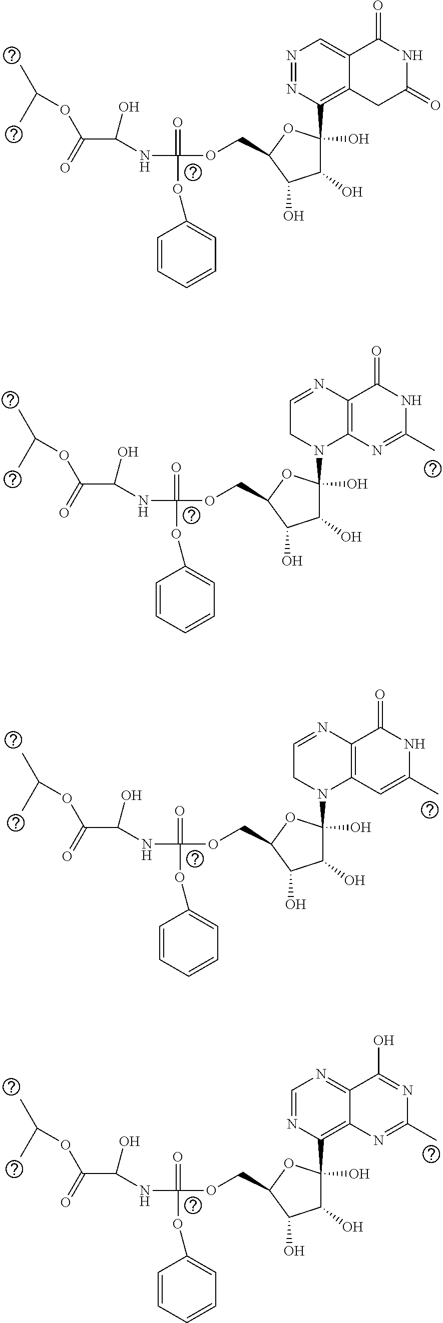

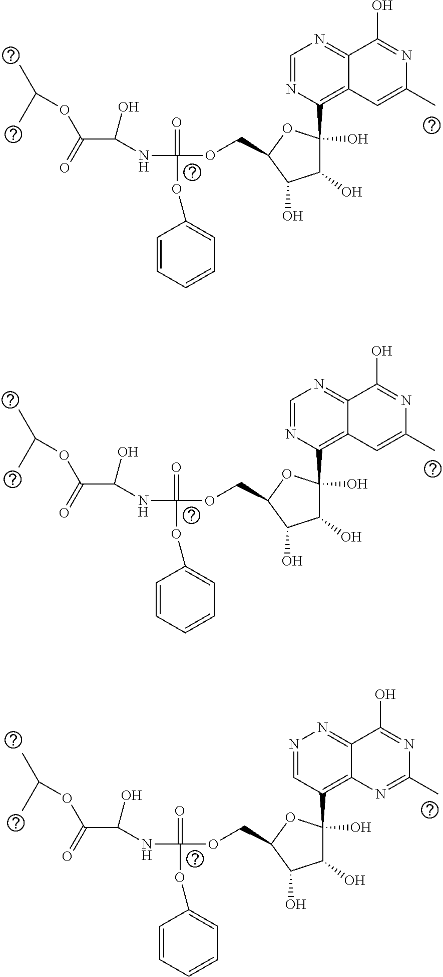

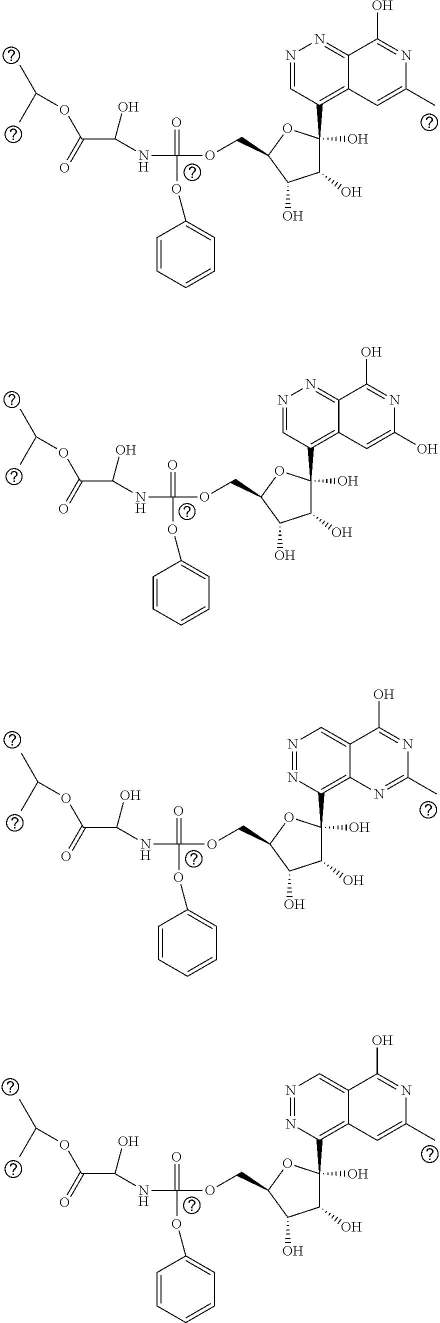

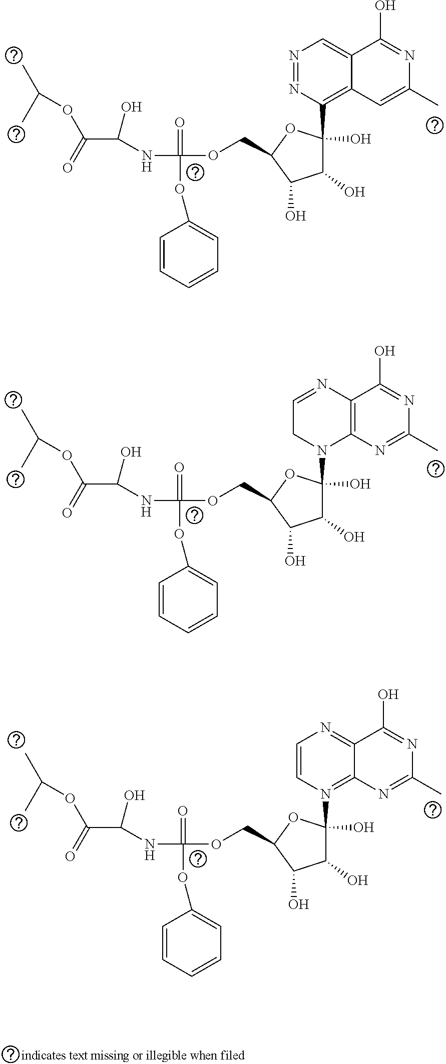

### Example 20. Molecular Docking, Binding Mode, and Binding Energy Analysis of Exemplary Compounds

The nucleoside analogue prodrugs described herein can be converted to active metabolites, 5′ ribose triphosphate (RTP), which base-pairs with DNA and/or RNA bases and gets incorporated into the viral DNA and/or viral RNA strand, resulting in viral mutagenesis. The structures of exemplary active metabolites and the corresponding exemplary prodrugs from which they are converted (i.e., Formulae 87, 84, 96, 101, 100, 102, 98, 97, 99, 88, and 86, where R₂₃ and R₂ are as defined above, e.g., paragraph no. 33 (b) on page 91; any one of Formulae XXI, XXII, 25, 25′, 26, 26′, 27, and 27′;

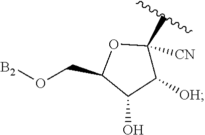

or the structures in Example 19 above) are shown below.

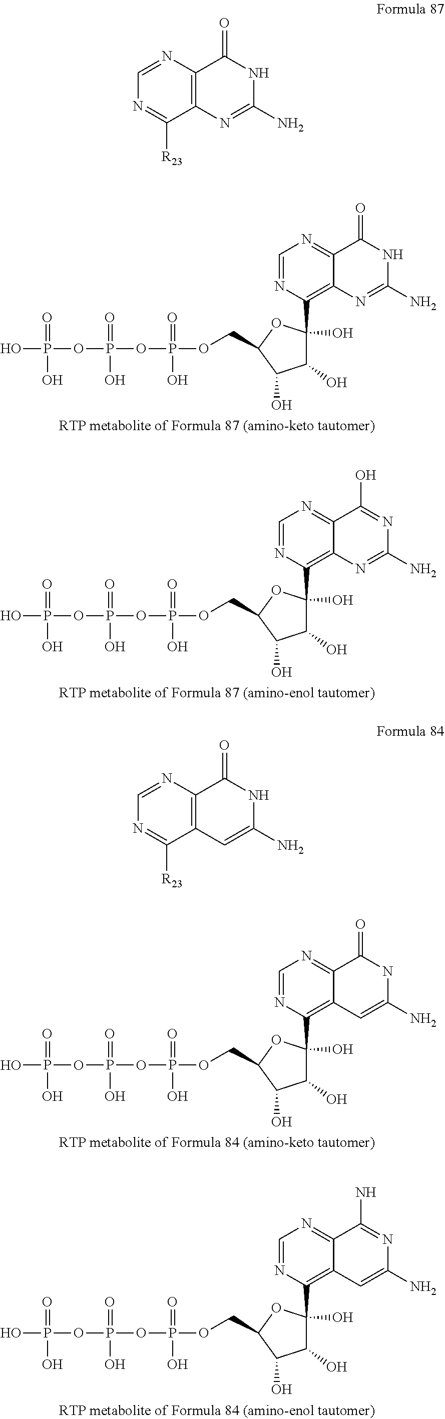

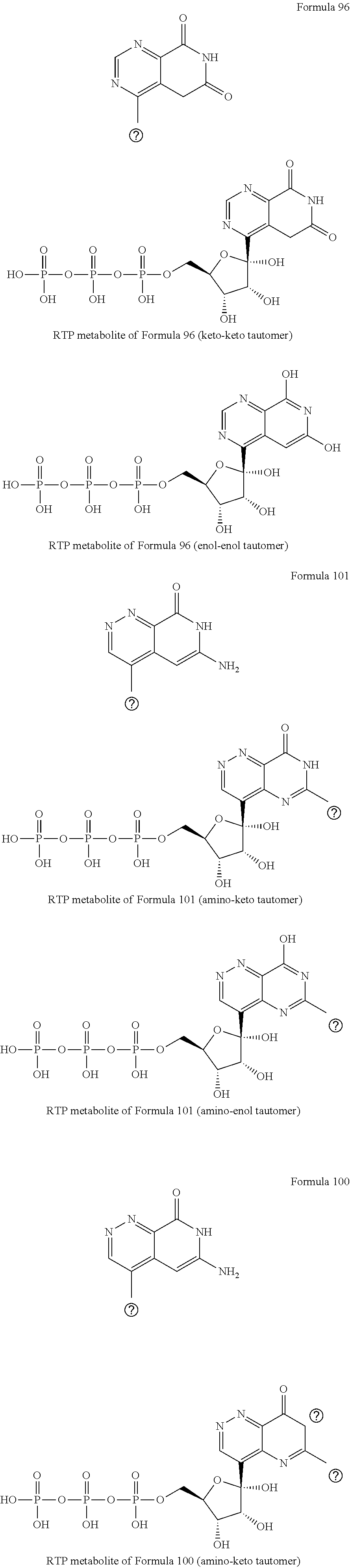

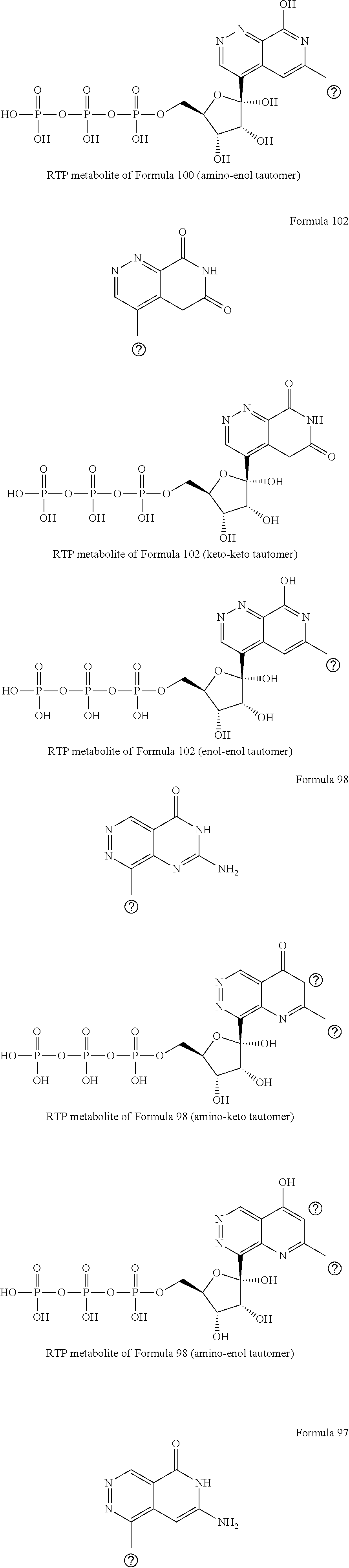

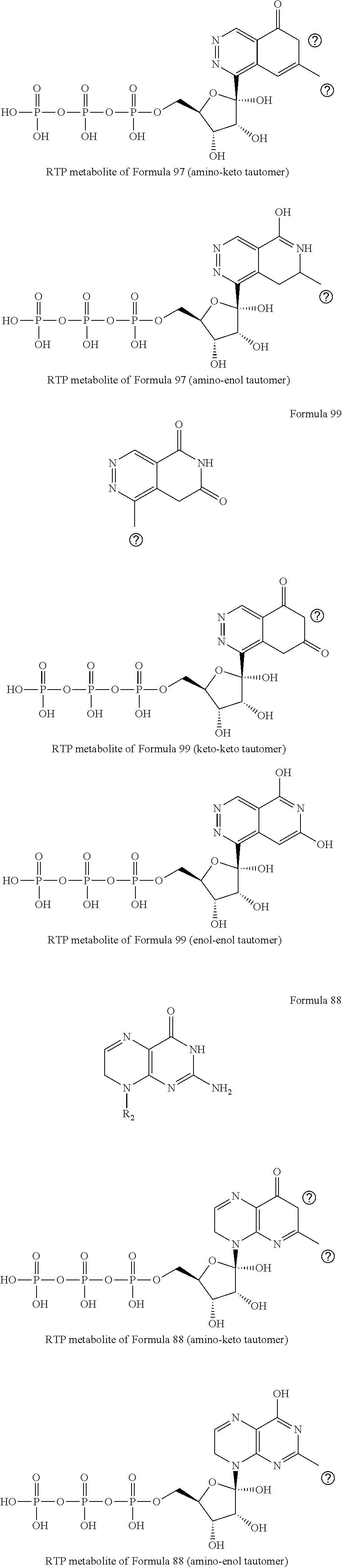

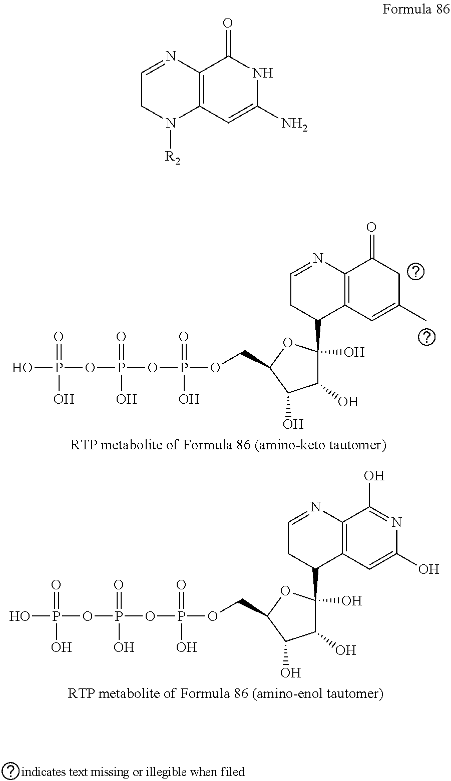

Although not illustrated above, it is understood that additional tautomer forms of these exemplary compounds and their active RTP metabolites are present. For example, in addition to the keto-keto and enol-enol tautomer shown above, the active RTP metabolites of the compounds of Formula 96, 102, and 99 can also be a keto-enol tautomer:

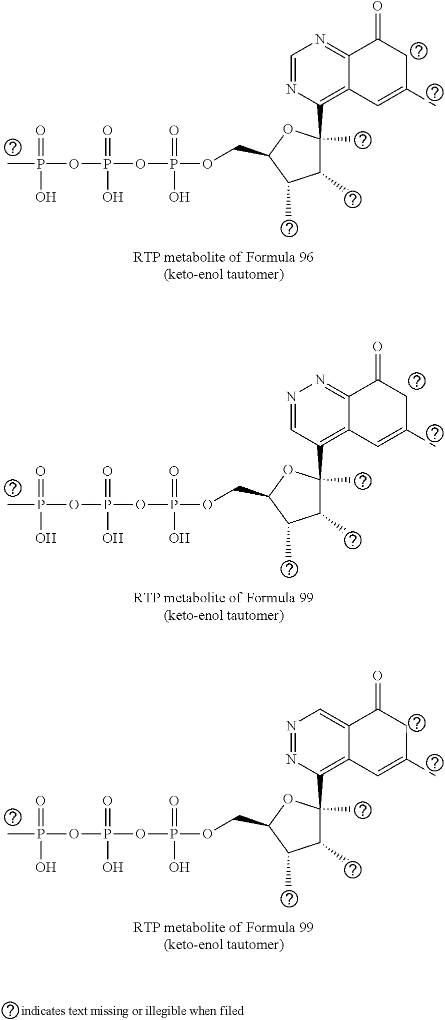

The active RTP metabolites of Formulae 87, 84, 96, 101, 100, 102, 98, 97, 99, 88, and 86, in different tautomer forms, were evaluated using molecular docking, binding mode, and binding energy analysis. The active RTP metabolites of reference compounds Guanosine and its amino-enol tautomer, Bemnifosbuvir and its amino-enol tautomer, Adenosine, and Remdesivir are used for comparison, the structures of which are shown below.

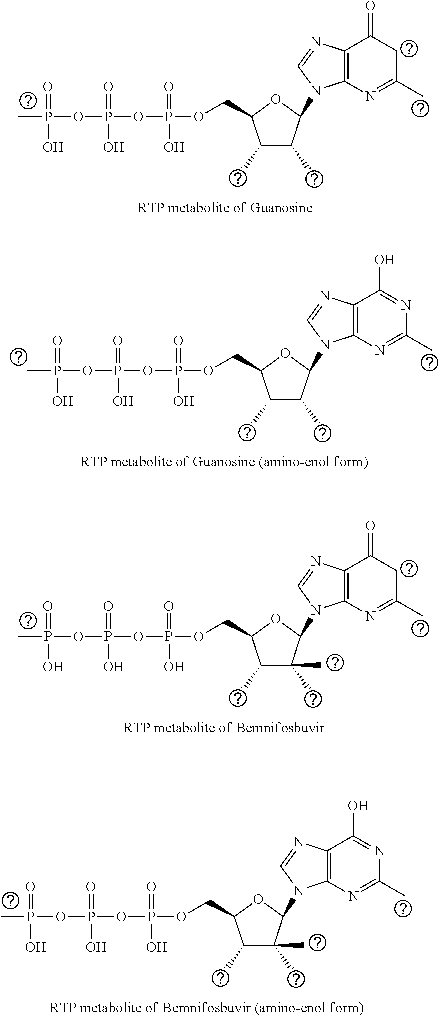

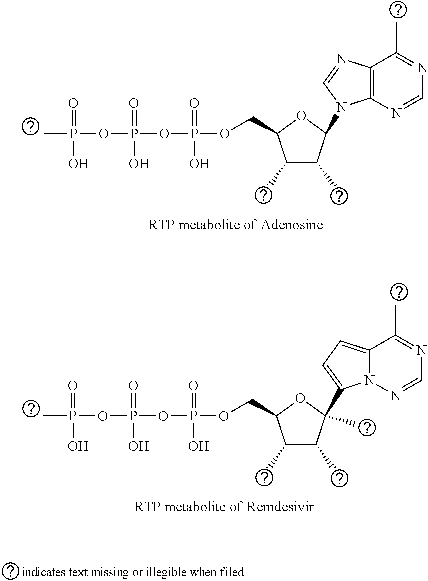 [SMILES: Cc1ncnc2c1ncn2[C@@H]1O[C@H](COP(=O)(O)OP(=O)(O)OP(C)(=O)O)[C@@H](C)[C@H]1C ; Cc1ncnn2c([C@]3(C)O[C@H](COP(=O)(O)OP(=O)(O)OP(C)(=O)O)[C@@H](C)[C@H]3C)ccc12]

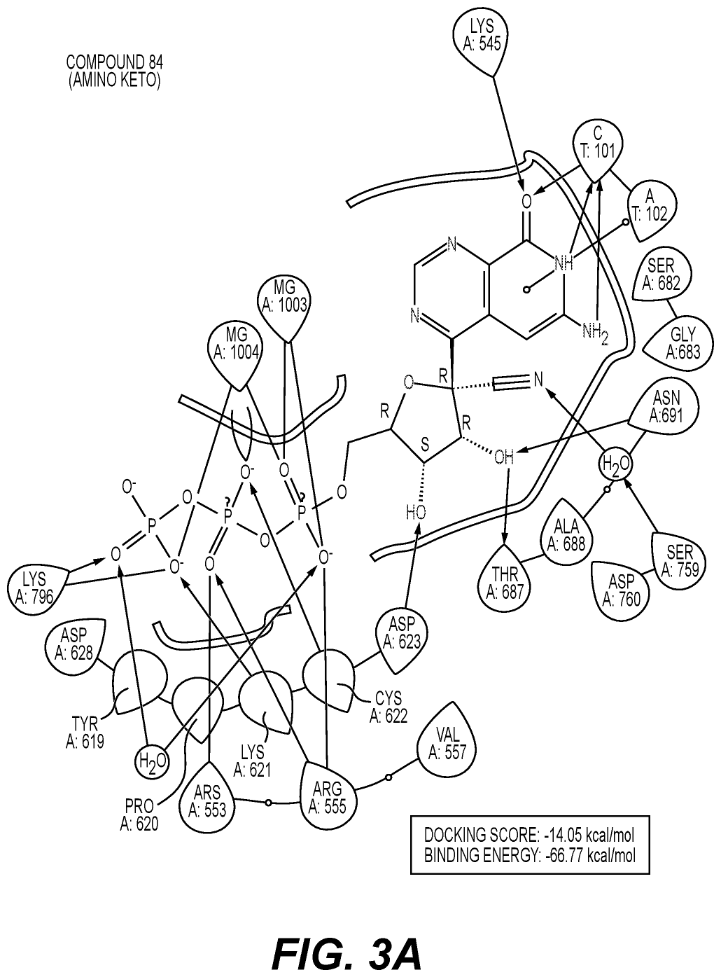

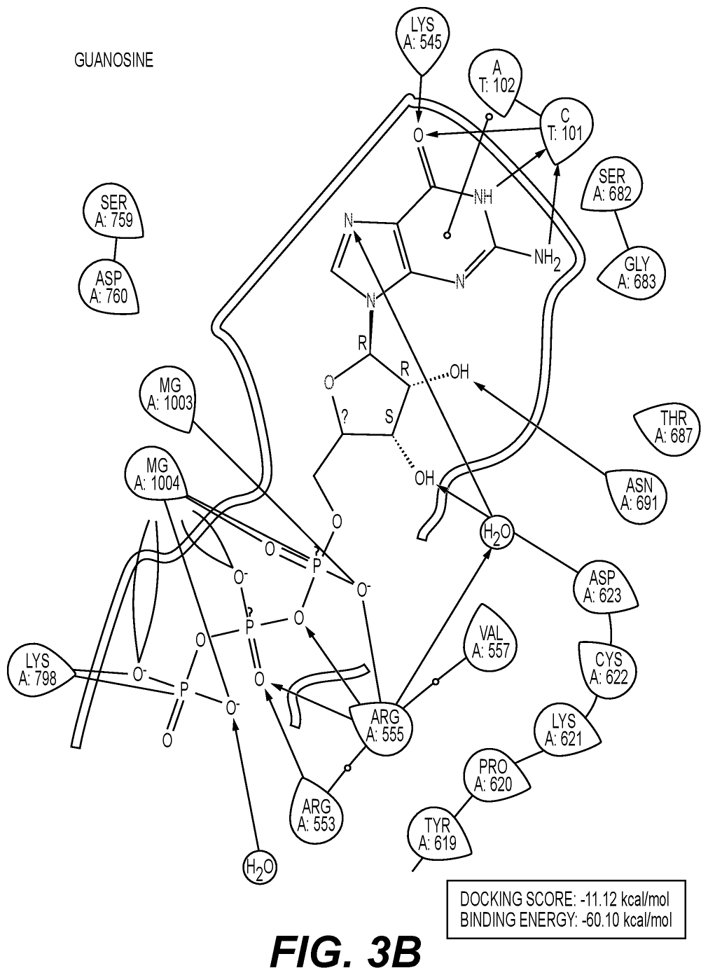

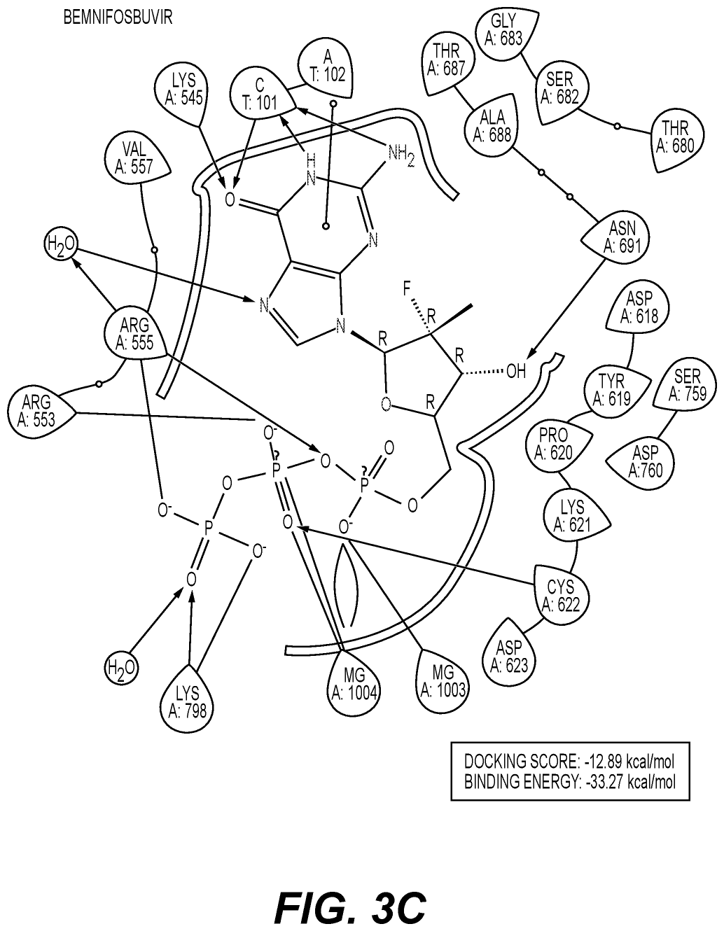

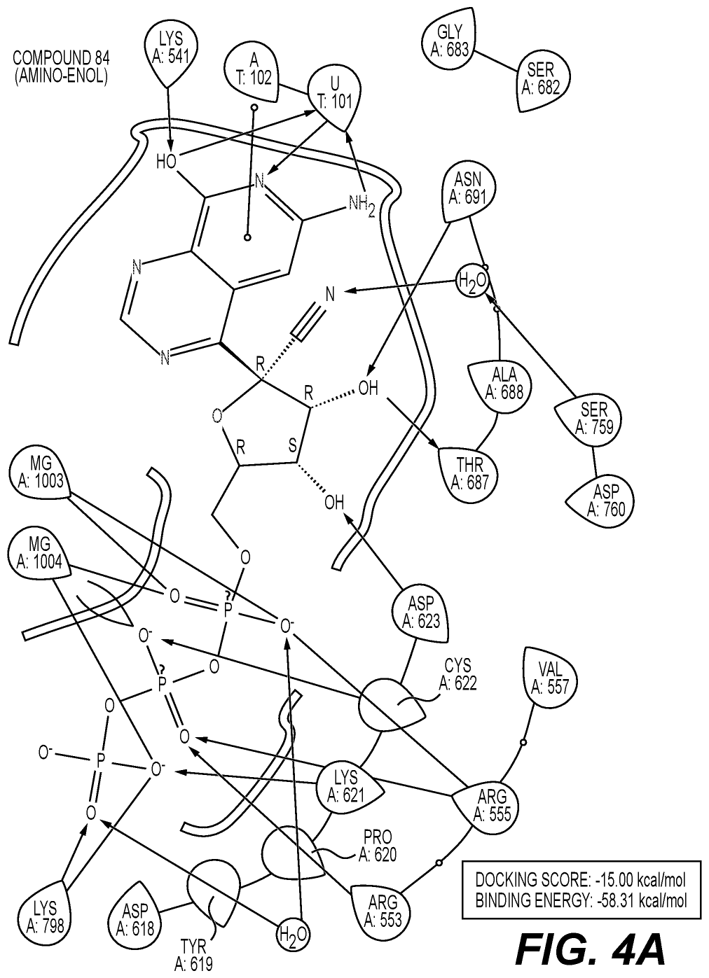

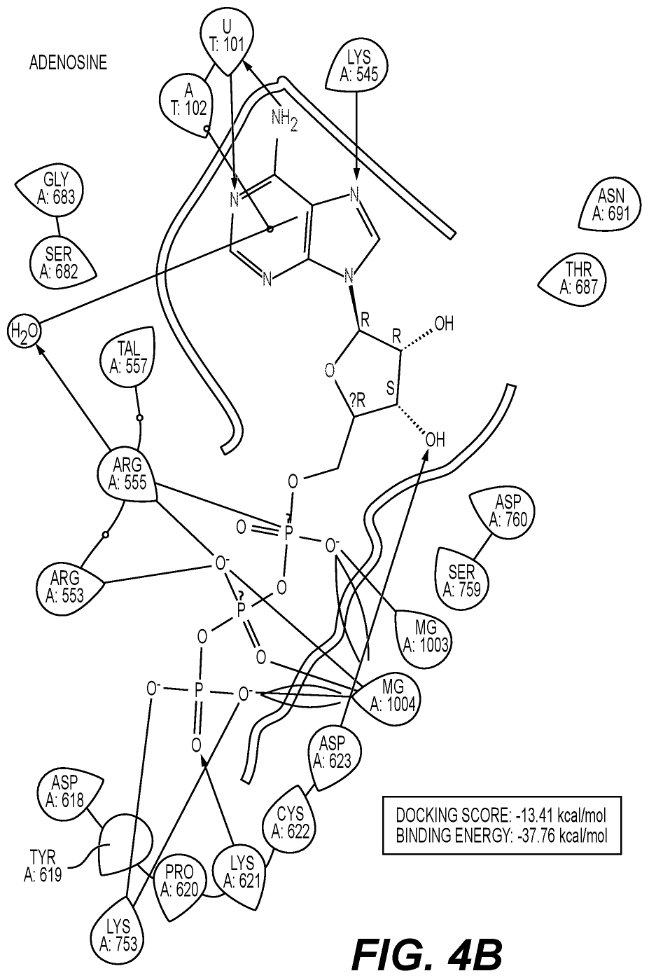

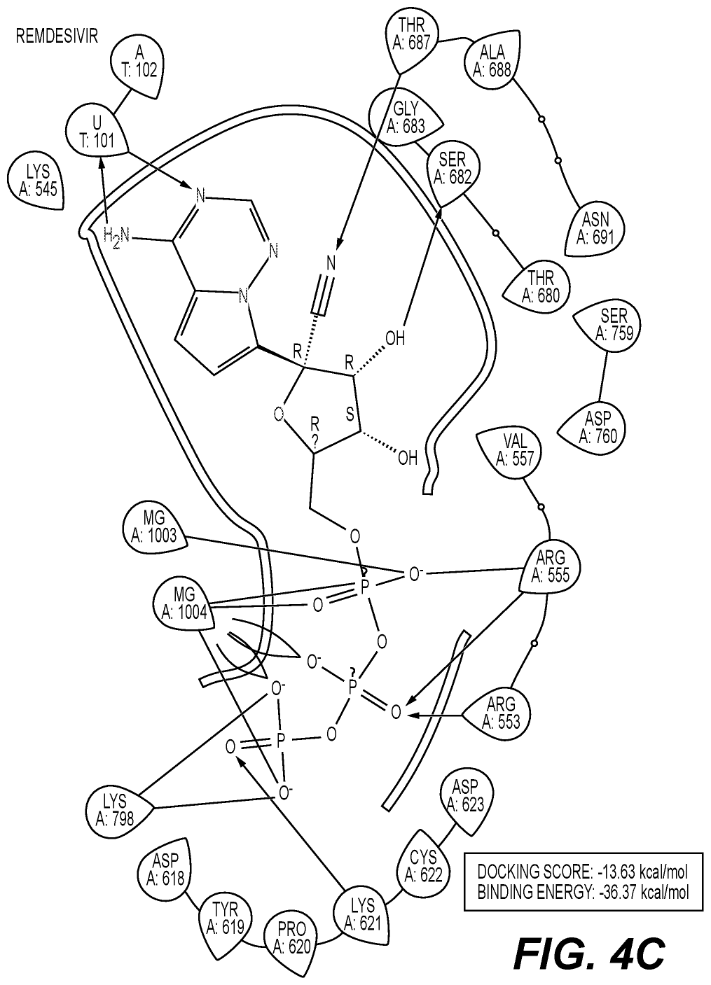

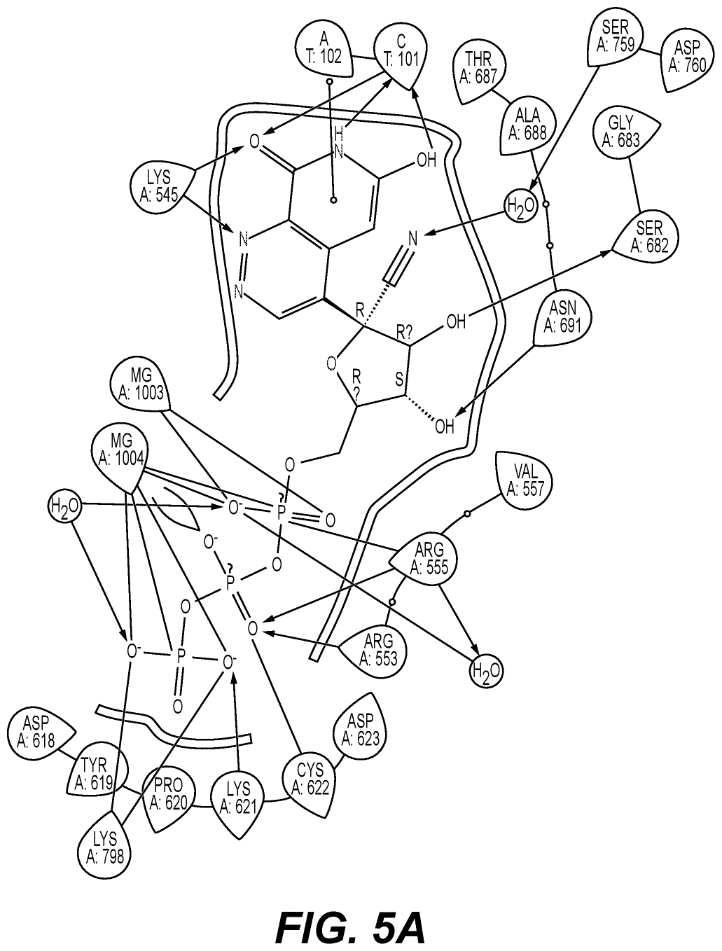

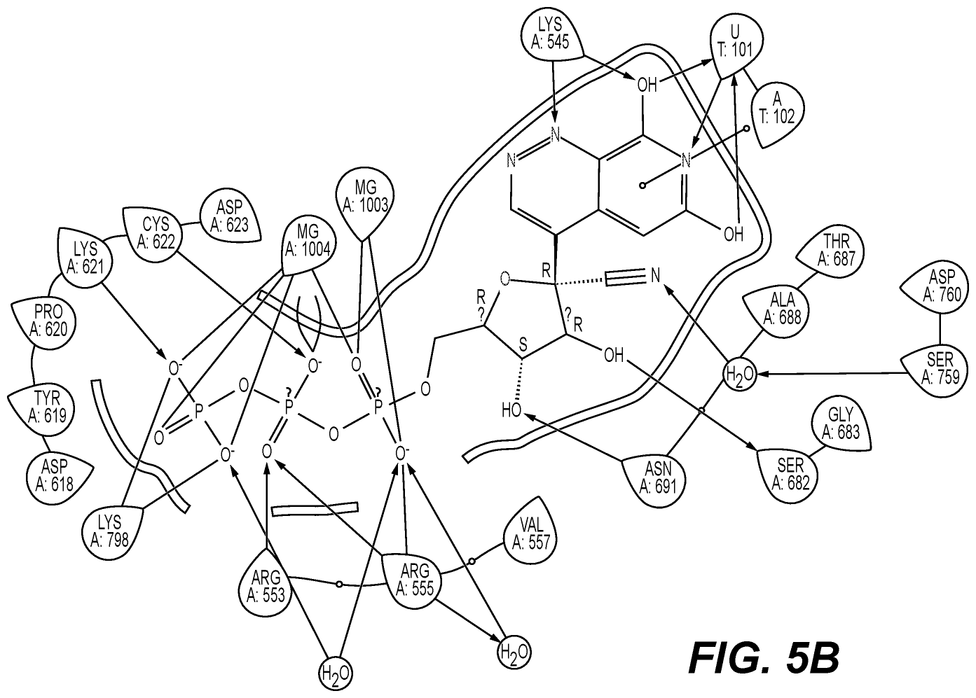

The bonding between the enol form of a triazolo-pyrazine compound and a nucleoside, cytidine, or an imino form the nucleoside are illustrated in FIGS. 1A and 1B, respectively. The bonding between a triazolo-pyrazine compound and the enol or keto form of a nucleoside, uridine, are illustrated in FIGS. 2A and 2B, respectively. The bonding between the amino-keto active RTP metabolite of the compound of Formula 84 and Cytidine MP within the SARS-CoV-2 RdRp, bonding between reference compound Guanosine and Cytidine MP within the SARS-CoV-2 RdRp, and bonding between reference compound Bemnifosbuvir (generated using the active metabolite, i.e., AT-9010) and Cytidine MP within the SARS-CoV-2 RdRp, are illustrated in FIGS. 3A-3C, respectively. The bonding between the amino-enol active RTP metabolite of the compound of Formula 84 and Uridine MP within the SARS-CoV-2 RdRp, bonding between reference compound Adenosine and Uridine MP within the SARS-CoV-2 RdRp, and bonding between reference compound Remdesivir TP (active metabolite) and the SARS-CoV-2 RdRp, are illustrated in FIGS. 4A-4C, respectively. Additionally, the bonding between the keto-enol active RTP metabolite of the compound of Formula 102 and Cytidine MP within the SARS-CoV-2 RdRp, and bonding between the enol-enol active RTP metabolite of the compound of Formula 102 and Uridine MP within the SARS-CoV-2 RdRp, are illustrated in FIGS. 5A and 5B, respectively.

The molecular docking, binding mode, and binding energy data are summarized in Tables 3-5. The binding energy reflects the affinity of the active metabolite for a target protein. The lower the free energy, the greater the affinity. Thus, the binding energies of the active metabolites of exemplary compounds described herein demonstrate their ability to compete with naturally occurring nucleosides and inflict chain termination.

As shown in Table 3, 9 out of the 11 active RTP metabolites show docking scores greater than the reference compounds, guanosine and bemnifosbuvir (generated using the active metabolite, i.e., AT-9010). Further, all 11 active RTP metabolites show binding energies exceeding bemnifosbuvir and 4 out of the 11 active RTP metabolites show binding energies greater than guanosine. These data demonstrate that the compounds described herein can mimic guanosine, with base pairing within the RdRp, leading to chain termination.

As shown in Table 4, 9 out of the 11 active RTP metabolites, in their tautomer forms, show docking scores matching the reference compounds, adenosine and remdesivir TP. Further, 9 out of the 11 active RTP metabolites show binding energies greater than adenosine and remdesivir. These data demonstrate that the compounds described herein, in different tautomer forms, can mimic adenosine, with ambiguous base pairing within the RdRp, leading to chain termination.

Table 5 shows the docking score and binding energy of the active RTP metabolite of an exemplary compound having a 5,6-cyclic ring structure, and the docking score and binding energy of an active RTP metabolite of an exemplary compound having a 6,6-cyclic ring structure. The active RTP metabolite structures are shown below. Without being bound to any theories, it is believed that the stereochemistry of the carbon atoms may play a role in binding of the 5,6-cyclic compounds to SARS-CoV-2 RdRp. For example, in Table 5, the exemplary 5,6-cyclic compound has a SRSR configuration on the ribose of the compound, which does not match that of Guanosine, and may affect the binding with SARS-CoV-2 RdRp.

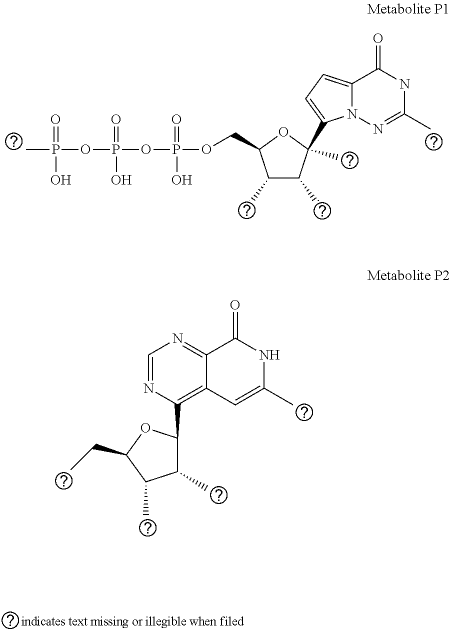

R is triphosphate

The data generated by different tautomer of a given compound demonstrate that the compounds described herein can mimic both guanosine and adenosine with similar or better binding energies, which can result in lower dose requirement and fewer side effects, and additional benefits, such as providing a barrier to viral resistance, the ability to target the nsp12NIRAN domain that is specific for guanosine (and analogues), and/or further enhanced effectiveness when compared with a protease inhibitor.

Unless defined otherwise, all technical and scientific terms used herein have the same meanings as commonly understood by one of skill in the art to which the disclosed invention belongs. Publications cited herein and the materials for which they are cited are specifically incorporated by reference. Those skilled in the art will recognize or be able to ascertain using no more than routine experimentation, many equivalents to the specific embodiments of the invention described herein. Such equivalents are intended to be encompassed by the following claims.

TABLE 3
Molecular Docking, Binding Mode and Binding Energy Analysis of Active RTP Metabolites of Exemplary Compounds*
Compared to Reference Compounds Guanosine and Bemnifosbuvir as TP (PDB:7UOB)

| | | | | | | | | | | | | | | | | Binding |
|---|---|---|---|---|---|---|---|---|---|---|---|---|---|---|---|---|
| | BINDING | | | | | | | | | | | | | | Docking | Energy |
| | | H-bonds with Amino Acid | | | | | | | | | | | | | Score | MM/GBSA |
| Structure | Base Pair\hat{ } | Arg553 | Arg555 | Asn691 | Asp623 | Cys622 | Lys545 | Lys621 | Lys798 | Ser682 | Ser759 | Thr687 | Ionic | Stacking | kcal/mol | |
| Guanosine | C 101 | ✓ | ✓ | ✓ | ✓ | x | ✓ | x | x | x | x | x | PO4: Mg²⁺, | π-π . . . | −11.12 | −60.1 |
| | | | | | | | | | | | | | Arg555, | A 102 (T) | | |
| | | | | | | | | | | | | | Lys798 | | | |
| Bemnifosbuvir | C 101 | x | x | ✓ | x | ✓ | ✓ | x | ✓ | x | x | x | PO4: Mg²⁺, | A 102 (T) | −12.89 | −33.27 |
| | | | | | | | | | | | | | Arg553- | | | |
| | | | | | | | | | | | | | Arg555, | | | |
| | | | | | | | | | | | | | Lys798 | | | |
| RTP of | C 101 | ✓ | ✓ | ✓ | ✓ | ✓ | ✓ | ✓ | x | x | ✓ | ✓ | PO4: Mg²⁺, | A 102 (T) | −12.97 | −70.36 |
| Formula 87 | | | | | | | | | | | | | Arg553, | | | |
| | | | | | | | | | | | | | Lys798 | | | |
| RTP of | C 101 | ✓ | ✓ | ✓ | ✓ | ✓ | ✓ | ✓ | ✓ | x | ✓ | ✓ | PO4: Mg²⁺, | A 102 (T) | −14.05 | −66.77 |
| Formula 84 | | | | | | | | | | | | | Arg555, | | | |
| | | | | | | | | | | | | | Lys798 | | | |
| RTP of | C 101 | x | x | ✓ | ✓ | x | ✓ | ✓ | ✓ | x | ✓ | ✓ | PO4: Mg²⁺, | A102 (T) | −14.64 | −71.87 |
| Formula 96 | | | | | | | | | | | | | Arg553, | | | |
| | | | | | | | | | | | | | Arg555, | | | |
| | | | | | | | | | | | | | Lys798 | | | |
| RTP of | C 101 | ✓ | ✓ | ✓ | x | ✓ | ✓ | ✓ | x | ✓ | ✓ | x | PO4: Mg²⁺, | A 102 (T) | −13.55 | −49.24 |
| Formula 101 | | | | | | | | | | | | | Arg555, | | | |
| | | | | | | | | | | | | | Lys798 | | | |
| RTP of | C 101 | ✓ | ✓ | ✓ | ✓ | ✓ | ✓ | ✓ | ✓ | x | ✓ | ✓ | PO4: Mg²⁺, | A 102 (T) | −13.79 | −59.35 |
| Formula 100 | | | | | | | | | | | | | Arg555, | | | |
| | | | | | | | | | | | | | Lys798 | | | |
| RTP of | C 101 | ✓ | ✓ | ✓ | x | ✓ | ✓ | ✓ | x | ✓ | ✓ | x | PO4: Mg²⁺, | A 102 (T) | −14.61 | −54.71 |
| Formula 102 | | | | | | | | | | | | | Arg555, | | | |
| | | | | | | | | | | | | | Lys798 | | | |
| RTP of | C 101 | ✓ | ✓ | ✓ | ✓ | ✓ | ✓ | ✓ | x | x | ✓ | ✓ | PO4: Mg²⁺, | A 102 (T) | −11.3 | −68.02 |
| Formula 98 | | | | | | | | | | | | | Arg555, | | | |
| | | | | | | | | | | | | | Lys798 | | | |
| RTP of | C 101 | ✓ | ✓ | ✓ | x | ✓ | ✓ | ✓ | ✓ | ✓ | ✓ | x | PO4: Mg²⁺, | A 102 (T) | −13.41 | −37.65 |
| Formula 97 | | | | | | | | | | | | | Arg555, | | | |
| | | | | | | | | | | | | | Lys798 | | | |
| RTP of | C 101 | ✓ | ✓ | ✓ | ✓ | ✓ | ✓ | ✓ | x | x | ✓ | ✓ | PO4: Mg²⁺, | A 102 (T) | −14.49 | −55.26 |
| Formula 99 | | | | | | | | | | | | | Arg555, | | | |
| | | | | | | | | | | | | | Lys798 | | | |
| RTP of | C 101 | ✓ | ✓ | ✓ | ✓ | ✓ | ✓ | ✓ | x | x | ✓ | ✓ | PO4: Mg²⁺, | A 102 (T) | −12.27 | −52.19 |
| Formula 88 | | | | | | | | | | | | | Arg555, | | | |
| | | | | | | | | | | | | | Lys798 | | | |
| RTP of | C 101 | ✓ | ✓ | ✓ | ✓ | ✓ | ✓ | ✓ | x | x | ✓ | ✓ | PO4: Mg²⁺, | A 102 (T) | −13.66 | −53.79 |
| Formula 86 | | | | | | | | | | | | | Arg555, | | | |
| | | | | | | | | | | | | | Lys798 | | | |

*amino-keto form or keto-keto form
\hat{ }C represents Cytidine MP

TABLE 4
Molecular Docking, Binding Mode and Binding Energy Analysis of Active RTP Metabolites of Exemplary Compounds**
Compared to Reference Compounds Guanosine^{#}, Bemnifosbuvir^{#}, Adenosine, and Remdesivir as TP (PDB:7UOB)

| | BINDING | | | | | | | | | | | | | | | Binding |
|---|---|---|---|---|---|---|---|---|---|---|---|---|---|---|---|---|
| | | | | | | | | | | | | | | | Docking | Energy |
| | | H-bonds with Amino Acid Ionic Stacking | | | | | | | | | | | | | Score | MM/GBSA |
| Structure | Base Pair\hat{ } | Arg553 | Arg555 | Asn691 | Asp623 | Cys622 | Lys545 | Lys621 | Lys798 | Ser682 | Ser759 | Thr687 | Ionic | Stacking | kcal/mol | |
| Adensosine | U 101 | x | x | x | ✓ | x | ✓ | ✓ | x | x | x | x | PO4: Mg²⁺, Arg553, | A 102 (T) | −13.41 | −37.76 |
| | | | | | | | | | | | | | Arg555, Lys798 | | | |
| Remdesivir | U 101 | ✓ | ✓ | x | x | x | x | ✓ | x | ✓ | x | ✓ | PO4: Mg²⁺, Arg555, | x | −13.63 | −36.37 |
| | | | | | | | | | | | | | Lys798 | | | |
| Guanosine | U 101 | x | ✓ | x | ✓ | x | ✓ | ✓ | x | ✓ | x | ✓ | PO4: Mg2+, Arg553, | π-π . . . | −10.94 | −50.3 |
| | | | | | | | | | | | | | Arg555 Lys621, | A102 (T) | | |
| | | | | | | | | | | | | | Lys798 | | | |
| Bemnifosbuvir | U 101 | ✓ | x | x | ✓ | ✓ | ✓ | ✓ | x | x | x | x | PO4: Mg2+, Arg553, | A 102 (T) | −10.55 | −24.78 |
| | | | | | | | | | | | | | Arg555 Lys621, | | | |
| | | | | | | | | | | | | | Lys798 | | | |
| RTP of | U 101 | ✓ | ✓ | ✓ | ✓ | ✓ | ✓ | ✓ | x | x | ✓ | ✓ | PO4: Mg2+, Arg555, | A 102 (T) | −13.36 | −58.5 |
| Formula 87 | | | | | | | | | | | | | Lys798 | | | |
| RTP of | U 101 | ✓ | ✓ | ✓ | ✓ | ✓ | ✓ | ✓ | ✓ | x | ✓ | ✓ | PO4: Mg2+, Arg555, | A 102 (T) | −15.00 | −58.31 |
| Formula 84 | | | | | | | | | | | | | Lys798 | | | |
| RTP of | U 101 | x | x | ✓ | ✓ | x | ✓ | ✓ | ✓ | x | ✓ | ✓ | PO4: Mg²⁺, Arg553, | A 102 (T) | −15.10 | −60.83 |
| Formula 96 | | | | | | | | | | | | | Arg555, Lys798 | | | |
| RTP of | U 101 | ✓ | ✓ | ✓ | x | ✓ | ✓ | ✓ | x | ✓ | ✓ | x | PO4: Mg²⁺, Arg555, | A 102 (T) | −13.39 | −36.53 |
| Formula 101 | | | | | | | | | | | | | Lys798 | | | |
| RTP of | U 101 | ✓ | ✓ | ✓ | ✓ | ✓ | ✓ | ✓ | ✓ | x | ✓ | ✓ | PO4: Mg²⁺, Arg555, | A 102 (T) | −14.67 | −54.37 |
| Formula 100 | | | | | | | | | | | | | Lys798 | | | |
| RTP of | U 101 | ✓ | ✓ | ✓ | x | ✓ | ✓ | ✓ | x | ✓ | ✓ | x | PO4: Mg²⁺, Arg555, | A 102 (T) | −14.46 | −44.35 |
| Formula 102 | | | | | | | | | | | | | Lys798 | | | |
| RTP of | U 101 | ✓ | ✓ | ✓ | ✓ | ✓ | ✓ | ✓ | x | x | ✓ | ✓ | PO4: Mg²⁺, Arg555, | A 102 (T) | −13.21 | −50.01 |
| Formula 98 | | | | | | | | | | | | | Lys798 | | | |
| RTP of | U 101 | ✓ | ✓ | ✓ | x | ✓ | ✓ | ✓ | ✓ | ✓ | ✓ | x | PO4: Mg²⁺, Arg555, | A 102 (T) | −14.40 | −31.79 |
| Formula 97 | | | | | | | | | | | | | Lys798 | | | |
| RTP of | U 101 | ✓ | ✓ | ✓ | ✓ | ✓ | ✓ | ✓ | x | x | ✓ | ✓ | PO4: Mg²⁺, Arg555, | A 102 (T) | −15.17 | −47.11 |
| Formula 99 | | | | | | | | | | | | | Lys798 | | | |
| RTP of | U 101 | ✓ | ✓ | ✓ | ✓ | ✓ | ✓ | ✓ | x | x | ✓ | ✓ | PO4: Mg²⁺, Arg555, | A 102 (T) | −14.13 | −48.59 |
| Formula 88 | | | | | | | | | | | | | Lys798 | | | |
| RTP of | U 101 | ✓ | ✓ | ✓ | ✓ | ✓ | ✓ | ✓ | x | x | ✓ | ✓ | PO4: Mg²⁺, Arg555, | A 102 (T) | −14.02 | −52.23 |
| Formula 86 | | | | | | | | | | | | | Lys798 | | | |

**amino-enol tautomer or enol-enol tautomer
^{#}amino-enol tautomer
\hat{ }U is for the Uridine MP

TABLE 5
Molecular Docking and Binding Energy Analysis of Monophosphate
Metabolite and Active RTP Metabolites of Exemplary Compounds

| | |
|---|---|
| | Binding Energy |
| Docking Score | MM/GBSA |

| | |
|---|---|
| Structure | kcal/mol |

| | | |
|---|---|---|
| Metabolite P1 | −12.854 | −52.36 |
| Metabolite P2 | −13.529 | −59.16 |

We claim:

1. A compound having the structure of 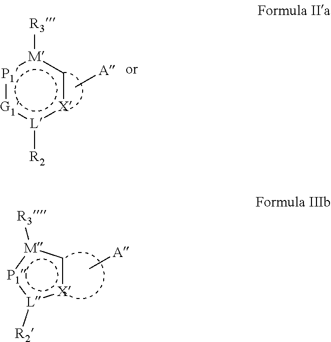
(a) wherein A″ are independently an unsubstituted heterocyclic group or a substituted heterocyclic group;
(b) wherein X′ is a carbon or a nitrogen;
(c) wherein L′, M′, L″, and M″ are independently a carbon or a nitrogen;
(d) wherein P₁′, P₁″, and G₁′ are independently CR₂₀ or NR₂₁, and R₂₀ and R₂₁ are independently absent, a hydrogen, a halogen, a nitrile group, a nitro group, an oxo group, an ester group, an unsubstituted aliphatic ester group, a substituted aliphatic ester group, an ether group, an unsubstituted aliphatic ether group, a substituted aliphatic ether group, an unsubstituted amino group, a substituted amino group, an unsubstituted aliphatic amino group, a substituted aliphatic amino group, an amide group, an aliphatic-amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted heterocyclic group, a substituted heterocyclic group, an unsubstituted heterocyclic-aliphatic group, or a substituted heterocyclic-aliphatic group;
(e) wherein R₂ and R₂′ are independently absent, a hydrogen, an unsubstituted aliphatic group, a substituted aliphatic group, an unsubstituted aryl group, a substituted aryl group, an unsubstituted aryl-aliphatic group, a substituted aryl-aliphatic group, an unsubstituted aliphatic ether group, or a substituted aliphatic ether group;
(f) wherein R₃′″ and R₃″″ are independently absent, a hydrogen, an unsubstituted aliphatic group, a substituted aliphatic group, an unsubstituted amide group, a substituted amide group, an unsubstituted aliphatic ester group, a substituted aliphatic ester group, an unsubstituted aliphatic ether group, a substituted aliphatic ether group, an unsubstituted heterocyclic group, a substituted heterocyclic group, an unsubstituted heterocyclic-aliphatic group, or a substituted heterocyclic-aliphatic group;
(g) A″ is 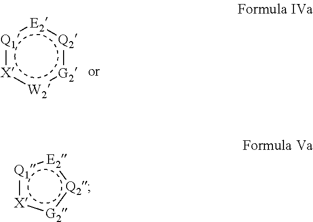
(gi) wherein X′ is a carbon or a nitrogen,
(gii) wherein E₂′, E₂″, G₂′, G₂″, Q₂′, Q₂″, and W₂′ are independently an oxygen, a sulfur, CR₂₀, or NR₂₁, and R₂₀ and R₂₁ are independently absent, a hydrogen, a halogen, a nitrile group, a nitro group, an oxo group, an ester group, an unsubstituted aliphatic ester group, a substituted aliphatic ester group, an ether group, an unsubstituted aliphatic ether group, a substituted aliphatic ether group, an unsubstituted amino group, a substituted amino group, an unsubstituted aliphatic amino group, a substituted aliphatic amino group, an amide group, an unsubstituted aliphatic-amide group, a substituted aliphatic-amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted heterocyclic group, a substituted heterocyclic group, an unsubstituted heterocyclic-aliphatic group, or a substituted heterocyclic-aliphatic group,
(giii) wherein Q₁′ and Q₁″ are carbon; and
(h) wherein the substituents are independently a phosphoramidate, a heterocyclic group, a halogen, a sulfonic acid, an azide group, a cyanate group, an isocyanate group, a nitrate group, a nitrile group, an isonitrile group, a nitrosooxy group, a nitroso group, a nitro group, an aldehyde group, an alkoxy group, an acyl halide group, a carboxylic acid group, a carboxylate group, an alkyl group, a heteroalkyl group, an alkenyl group, a heteroalkenyl group, an alkynyl group, a heteroalkynyl group, an aryl group, a heteroaryl group, polyaryl group, a heteropoly aryl group, a alkyl aryl group, an amino group, an ester group, a hydroxyl group, a thiol group, a sulfonyl group, an ester group, an aliphatic ester group, an amide group, an aliphatic amide group, an azo group, an acyl group, a carbonyl group, a carbonate ester group, an ether group, an aminooxy group, or a hydroxyamino group.

2. The compound of claim 1, wherein the compound has the structure of 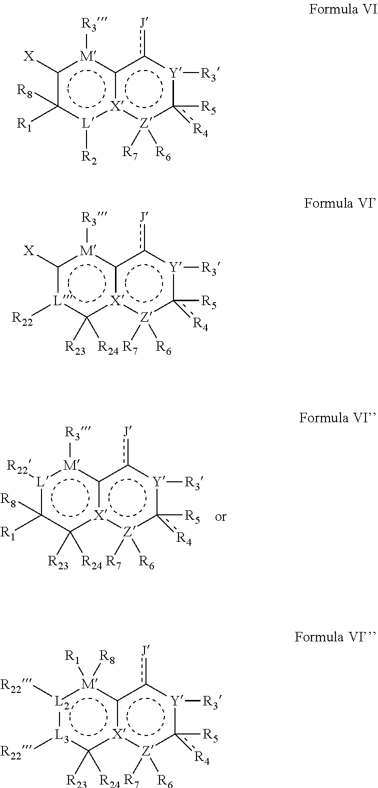
(a) wherein X′ is a carbon or a nitrogen;
(b) wherein L′, M′, L′″, and L₁-L₃ are carbon or nitrogen;
(c) wherein X is a hydrogen, a halogen, a nitrile group, a nitro group, an ester group, an unsubstituted aliphatic ester group, a substituted aliphatic ester group, an unsubstituted amino group, a substituted amino group, an amide group, an unsubstituted aliphatic-amide group, a substituted aliphatic-amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted heterocyclic group, a substituted heterocyclic group, an unsubstituted heterocyclic-aliphatic group, or a substituted heterocyclic-aliphatic group;
(d) wherein Y′ is a nitrogen;
(e) wherein Z′ is a an oxygen, a nitrogen, or a carbon;
(f) wherein J′ is an oxygen or OR₃;
(g) wherein R₁ and R₂₃ are independently a hydrogen, a halogen, a nitrile group, a nitro group, an ester group, an unsubstituted aliphatic ester group, a substituted aliphatic ester group, an unsubstituted amino group, a substituted amino group, an unsubstituted aliphatic amino group, a substituted aliphatic amino group, an amide group, an unsubstituted aliphatic amide group, a substituted aliphatic amide group, an azide group, an unsubstituted aliphatic group, a substituted aliphatic group, an unsubstituted aryl group, a substituted aryl group, an unsubstituted aryl-aliphatic group, a substituted aryl-aliphatic group, an ether group, an unsubstituted aliphatic ether group, a substituted aliphatic ether group;
(h) wherein R₂, R₂₂, R₂₂′, R₂₂″, and R₂₂′″ are independently absent, a hydrogen, an unsubstituted aliphatic group, a substituted aliphatic group, an unsubstituted aryl group, a substituted aryl group, an unsubstituted aryl-aliphatic group, a substituted aryl-aliphatic group, an unsubstituted aliphatic ether group, or a substituted aliphatic ether group;
(i) wherein R₃, R₃′, and R₃′″ are independently absent, a hydrogen, an unsubstituted aliphatic group, a substituted aliphatic group, an unsubstituted aliphatic ester group, a substituted aliphatic ester group, an unsubstituted aliphatic ether group, a substituted aliphatic ether group, an unsubstituted heterocyclic group, a substituted heterocyclic group, an unsubstituted heterocyclic-aliphatic group, or a substituted heterocyclic-aliphatic group;
(j) wherein R₈ and R₂₄ are independently absent, a hydrogen, a halogen, a nitrile group, an ester group, an unsubstituted aliphatic ester group, a substituted aliphatic ester group, an unsubstituted amino group, a substituted amino group, an unsubstituted aliphatic amino group, a substituted aliphatic amino group, an amide group, a substituted aliphatic-amide group, an unsubstituted aliphatic-amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted aliphatic ether group, a substituted aliphatic ether group, un unsubstituted heterocyclic group, a substituted heterocyclic group, an unsubstituted heterocyclic-aliphatic group, or a substituted heterocyclic-aliphatic group; and
(k) wherein R₄, R₅, R₆, and R₇ are independently absent, a hydrogen, a halogen, a nitrile group, an oxo group, an ester group, an unsubstituted aliphatic ester group, a substituted aliphatic ester group, an unsubstituted amino group, a substituted amino group, an unsubstituted aliphatic-amino group, a substituted aliphatic-amino group, an amide group, an unsubstituted aliphatic-amide group, a substituted aliphatic-amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted aliphatic ether group, a substituted aliphatic ether group, un unsubstituted heterocyclic group, a substituted heterocyclic group, an unsubstituted heterocyclic-aliphatic group, or a substituted heterocyclic-aliphatic group;
(l) wherein the substituents are as defined in the base claim(s).

3. The compound of claim 2, wherein:
R₁ or R₂ of Formula VI, R₂₂ or R₂₃ of Formula VI′, R₂₃ or R₁ of Formula VI″, and R₂₃, R₂₂″, or R₂₂′″ of Formula VI′″ is a substituted an unsubstituted aliphatic ester group, a substituted aliphatic ester group, an unsubstituted aliphatic ether group, or a substituted aliphatic ether group, preferably an unsubstituted or substituted aliphatic ether, such as an unsubstituted C₁-C₁₄ aliphatic ether or a substituted C₁-C₁₄ aliphatic ether, or an unsubstituted C₁-C₆ aliphatic ether or a substituted C₁-C₆ aliphatic ether; or
R₄ and R₅ are independently absent, hydrogen, an oxo group, an unsubstituted amino group, a substituted amino group, an unsubstituted aliphatic-amino group, or a substituted aliphatic-amino group; or
when J′ is OR₃, R₃ is a hydrogen, a substituted or unsubstituted aliphatic group, or a substituted or unsubstituted amino group; or
L′, M′, L′″, and L₁-L₃ are nitrogen; X′ is carbon; Z′ is a nitrogen or a carbon; and R₆ and R₇ are independently absent or hydrogen; or
a combination thereof.

4. The compound of claim 1, wherein the compound has the structure of

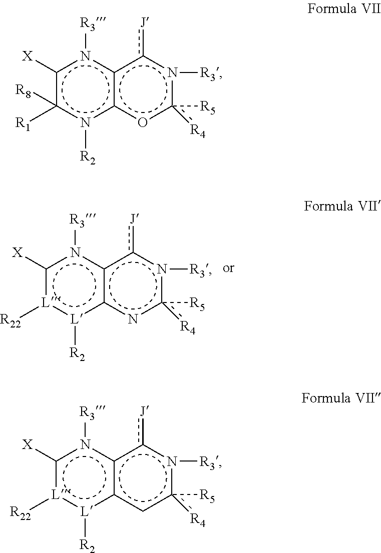

5. The compound of claim 1, wherein the compound has the structure of 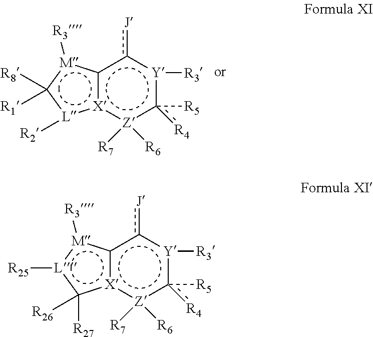
(a) wherein X′ is a carbon or a nitrogen;
(b) wherein L″, M″, and L″″ are independently a carbon or a nitrogen;
(c) wherein Y′ is a nitrogen;
(d) wherein Z′ is a an oxygen, a nitrogen, or a carbon;
(e) wherein J′ is an oxygen or OR₃;
(f) wherein R₁′ and R₂₆ are independently a hydrogen, a halogen, a nitrile group, a nitro group, an ester group, an unsubstituted aliphatic ester group, a substituted aliphatic ester group, an unsubstituted amino group, a substituted amino group, an unsubstituted aliphatic amino group, a substituted aliphatic amino group, an amide group, an unsubstituted aliphatic amide group, a substituted aliphatic amide group, an azide group, an unsubstituted aliphatic group, a substituted aliphatic group, an unsubstituted aryl group, a substituted aryl group, an unsubstituted aryl-aliphatic group, a substituted aryl-aliphatic group, an ether group, an unsubstituted aliphatic ether group, a substituted aliphatic ether group;
(g) wherein R₂′ and R₂₅ are independently absent, a hydrogen, an unsubstituted aliphatic group, a substituted aliphatic group, an unsubstituted aryl group, a substituted aryl group, an unsubstituted aryl-aliphatic group, a substituted aryl-aliphatic group, an unsubstituted aliphatic ether group, or a substituted aliphatic ether group;
(h) wherein R₃, R₃′, and R₃″″ is absent, a hydrogen, an unsubstituted aliphatic group, a substituted aliphatic group, an unsubstituted amide group, a substituted amide group, an unsubstituted aliphatic ester group, a substituted aliphatic ester group, an unsubstituted aliphatic ether group, a substituted aliphatic ether group, an unsubstituted heterocyclic group, a substituted heterocyclic group, an unsubstituted heterocyclic-aliphatic group, or a substituted heterocyclic-aliphatic group;
(i) wherein R₈′ and R₂₇ are independently absent, a hydrogen, a halogen, a nitrile group, an ester group, an unsubstituted aliphatic ester group, a substituted aliphatic ester group, an unsubstituted amino group, a substituted amino group, an unsubstituted aliphatic amino group, a substituted aliphatic amino group, an amide group, a substituted aliphatic-amide group, an unsubstituted aliphatic-amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted aliphatic ether group, a substituted aliphatic ether group, un unsubstituted heterocyclic group, a substituted heterocyclic group, an unsubstituted heterocyclic-aliphatic group, or a substituted heterocyclic-aliphatic group;
(j) wherein R₄, R₅, R₆, and R₇ are independently absent, a hydrogen, a halogen, a nitrile group, an oxo group, an ester group, an unsubstituted aliphatic ester group, a substituted aliphatic ester group, an unsubstituted amino group, a substituted amino group, an unsubstituted aliphatic-amino group, a substituted aliphatic-amino group, an amide group, an unsubstituted aliphatic-amide group, a substituted aliphatic-amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted aliphatic ether group, a substituted aliphatic ether group, un unsubstituted heterocyclic group, a substituted heterocyclic group, an unsubstituted heterocyclic-aliphatic group, or a substituted heterocyclic-aliphatic group;
(k) wherein the substituents are as defined in the base claim(s).

6. The compound of claim 5, wherein:
R₁′ or R₂′ of Formula XI and R₂₅ or R₂₆ of Formula XI′ is a substituted an unsubstituted aliphatic ester group, a substituted aliphatic ester group, an unsubstituted aliphatic ether group, or a substituted aliphatic ether group, preferably an unsubstituted or substituted aliphatic ether, such as an unsubstituted C₁-C₁₄ aliphatic ether or a substituted C₁-C₁₄ aliphatic ether, or an unsubstituted C₁-C₆ aliphatic ether or a substituted C₁-C₆ aliphatic ether; or
R₄ and R₅ are independently absent, hydrogen, an oxo group, an unsubstituted amino group, a substituted amino group, an unsubstituted aliphatic-amino group, or a substituted aliphatic-amino group; or
when J′ is OR₃, R₃ is a hydrogen, a substituted or unsubstituted aliphatic group, or a substituted or unsubstituted amino group; or
a combination thereof.

7. The compound of claim 1, wherein the compound has the structure of

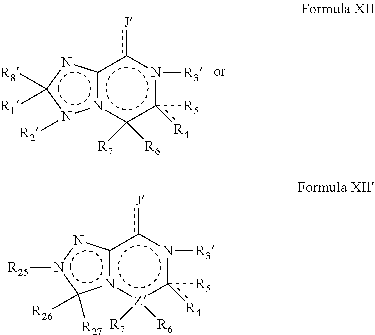

8. The compound of claim 1, wherein the compound has the structure of: 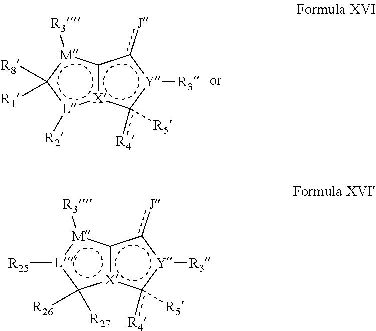
(a) wherein X′ is a carbon or a nitrogen;
(b) wherein L″, M″, and L″″ are independently a carbon or a nitrogen;
(c) wherein Y″ is a nitrogen;
(d) wherein J″ is an oxygen or OR₃;
(e) wherein R₁′ and R₂₆ are independently a hydrogen, a halogen, a nitrile group, a nitro group, an ester group, an unsubstituted aliphatic ester group, a substituted aliphatic ester group, an unsubstituted amino group, a substituted amino group, an unsubstituted aliphatic amino group, a substituted aliphatic amino group, an amide group, an unsubstituted aliphatic amide group, a substituted aliphatic amide group, an azide group, an unsubstituted aliphatic group, a substituted aliphatic group, an unsubstituted aryl group, a substituted aryl group, an unsubstituted aryl-aliphatic group, a substituted aryl-aliphatic group, an ether group, an unsubstituted aliphatic ether group, a substituted aliphatic ether group;
(f) wherein R₂′ and R₂₅ are independently absent, a hydrogen, an unsubstituted aliphatic group, a substituted aliphatic group, an unsubstituted aryl group, a substituted aryl group, an unsubstituted aryl-aliphatic group, a substituted aryl-aliphatic group, an unsubstituted aliphatic ether group, or a substituted aliphatic ether group;
(g) wherein R₃, R₃″, and R₃″″ is absent, a hydrogen, an unsubstituted aliphatic group, a substituted aliphatic group, an unsubstituted amide group, a substituted amide group, an unsubstituted aliphatic ester group, a substituted aliphatic ester group, an unsubstituted aliphatic ether group, a substituted aliphatic ether group, an unsubstituted heterocyclic group, a substituted heterocyclic group, an unsubstituted heterocyclic-aliphatic group, or a substituted heterocyclic-aliphatic group;
(h) wherein R₈′ and R₂₇ are independently absent, a hydrogen, a halogen, a nitrile group, an ester group, an unsubstituted aliphatic ester group, a substituted aliphatic ester group, an unsubstituted amino group, a substituted amino group, an unsubstituted aliphatic amino group, a substituted aliphatic amino group, an amide group, a substituted aliphatic-amide group, an unsubstituted aliphatic-amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted aliphatic ether group, a substituted aliphatic ether group, un unsubstituted heterocyclic group, a substituted heterocyclic group, an unsubstituted heterocyclic-aliphatic group, or a substituted heterocyclic-aliphatic group;
(i) wherein R₄ and R₅ are independently absent, a hydrogen, a halogen, a nitrile group, an oxo group, an ester group, an unsubstituted aliphatic ester group, a substituted aliphatic ester group, an unsubstituted amino group, a substituted amino group, an unsubstituted aliphatic-amino group, a substituted aliphatic-amino group, an amide group, an unsubstituted aliphatic-amide group, a substituted aliphatic-amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted aliphatic ether group, a substituted aliphatic ether group, un unsubstituted heterocyclic group, a substituted heterocyclic group, an unsubstituted heterocyclic-aliphatic group, or a substituted heterocyclic-aliphatic group;
(j) wherein the substituents are as defined in the base claim(s).

9. The compound of claim 8, wherein:
R₁′ or R₂′ of Formula XVI and R₂₅ or R₂₆ of Formula XVI′ is a substituted an unsubstituted aliphatic ester group, a substituted aliphatic ester group, an unsubstituted aliphatic ether group, or a substituted aliphatic ether group, preferably an unsubstituted or substituted aliphatic ether, such as an unsubstituted C₁-C₁₄ aliphatic ether or a substituted C₁-C₁₄ aliphatic ether, or an unsubstituted C₁-C₆ aliphatic ether or a substituted C₁-C₆ aliphatic ether; or
R₄ and R₅ are independently absent, hydrogen, an oxo group, an unsubstituted amino group, a substituted amino group, an unsubstituted aliphatic-amino group, or a substituted aliphatic-amino group; or
when J″ is OR₃, R₃ is a hydrogen, a substituted or unsubstituted aliphatic group, or a substituted or unsubstituted amino group; or
a combination thereof.

10. The compound of claim 1, wherein the compound has the structure of

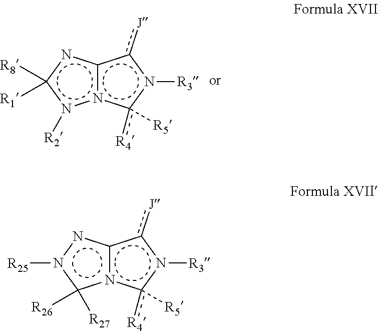

11. The compound of claim 1, wherein the compound has the structure of 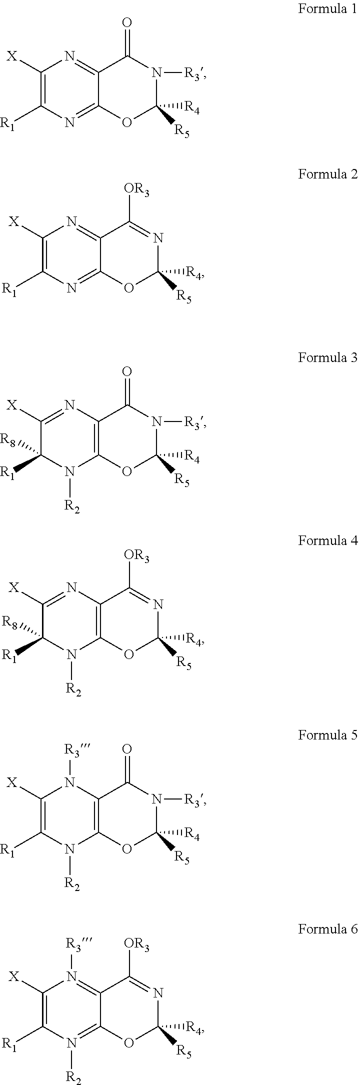 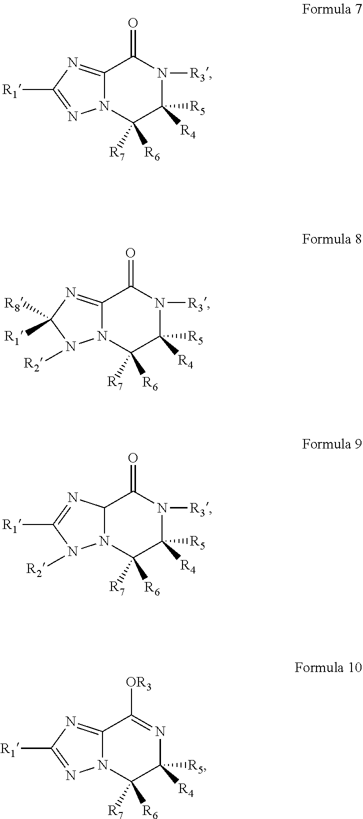 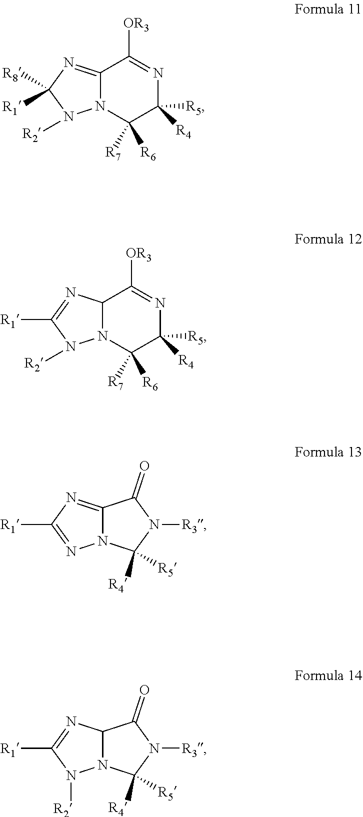 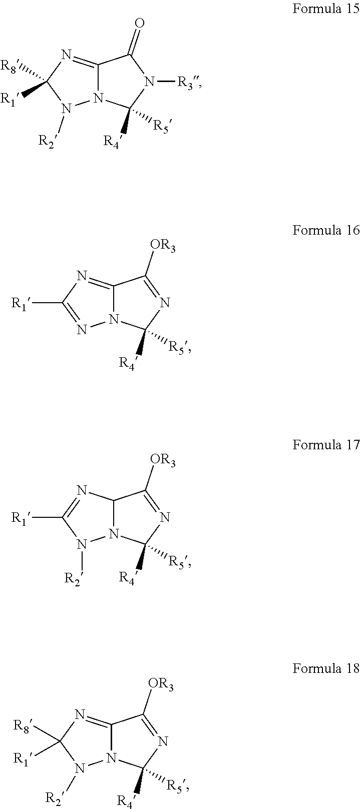 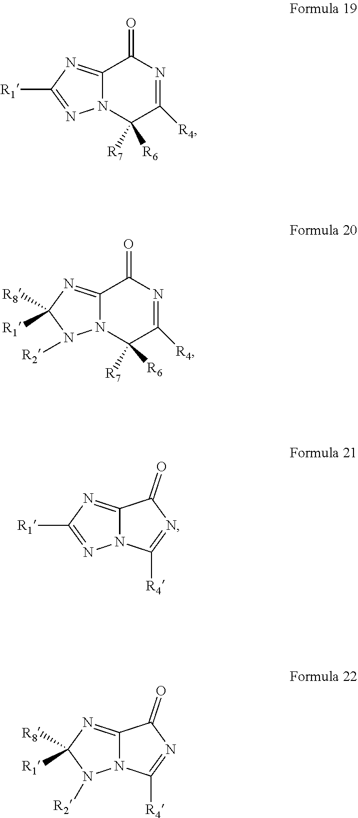 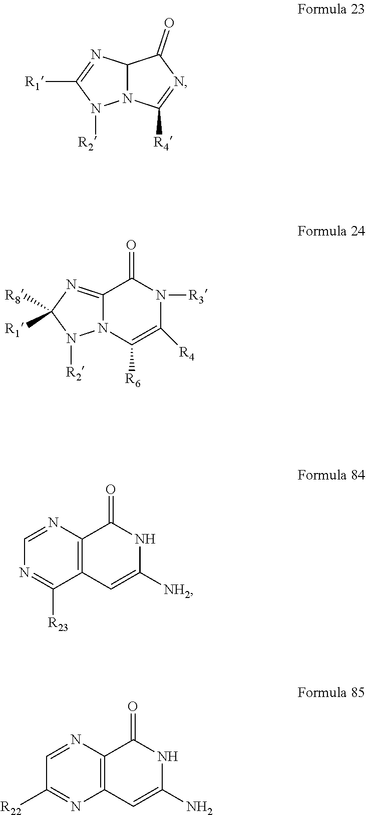 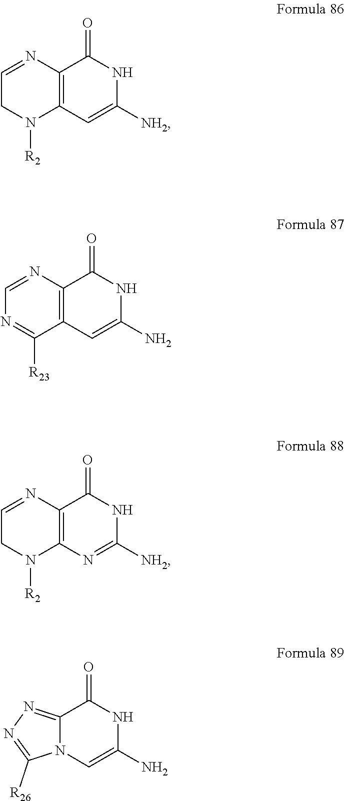 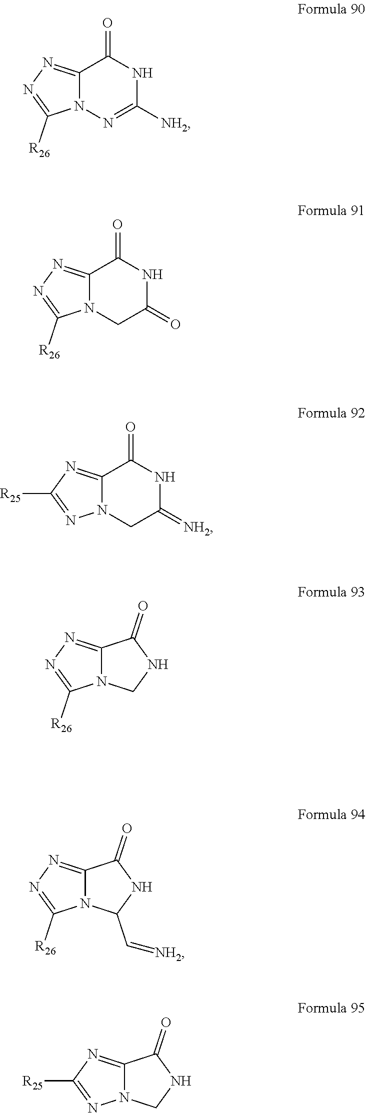 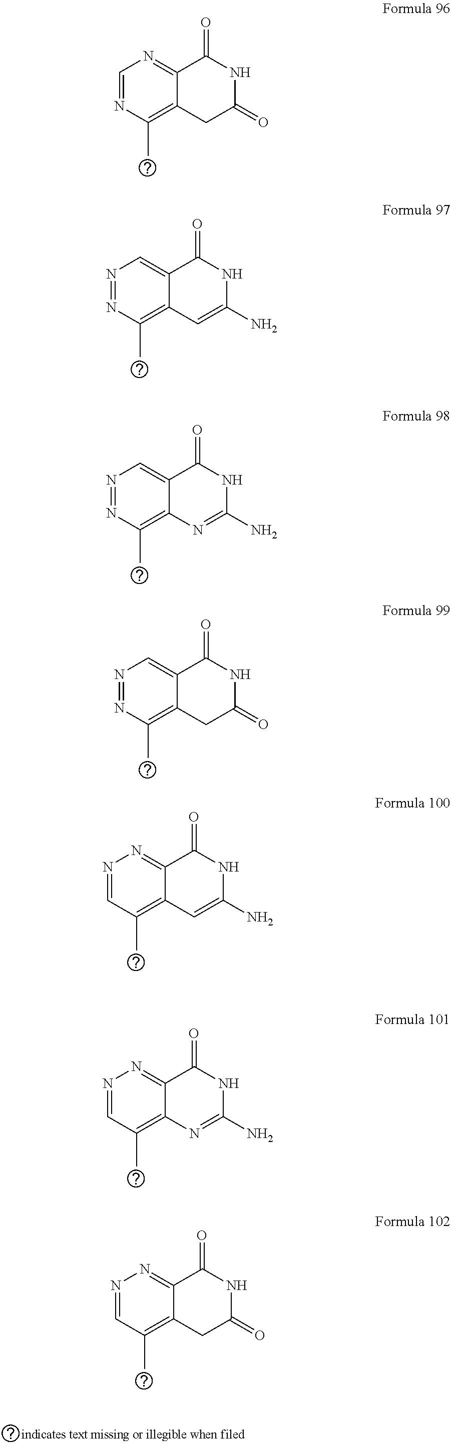
(a) wherein X is a hydrogen, a halogen, a nitrile group, a nitro group, an ester group, an unsubstituted aliphatic ester group, a substituted aliphatic ester group, an unsubstituted amino group, a substituted amino group, an amide group, an unsubstituted aliphatic-amide group, a substituted aliphatic-amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted heterocyclic group, a substituted heterocyclic group, an unsubstituted heterocyclic-aliphatic group, or a substituted heterocyclic-aliphatic group;
(b) wherein R₁, R₁′, R₂, R₂′, R₂₂, R₂₃, R₂₅, and R₂₆ are independently a hydrogen, a halogen, a nitrile group, a nitro group, an ester group, an unsubstituted aliphatic ester group, a substituted aliphatic ester group, an unsubstituted amino group, a substituted amino group, an unsubstituted aliphatic amino group, a substituted aliphatic amino group, an amide group, an unsubstituted aliphatic amide group, a substituted aliphatic amide group, an azide group, an unsubstituted aliphatic group, a substituted aliphatic group, an unsubstituted aryl group, a substituted aryl group, an unsubstituted aryl-aliphatic group, a substituted aryl-aliphatic group, an ether group, an unsubstituted aliphatic ether group, a substituted aliphatic ether group;
(c) wherein R₃, R₃′, R₃″, and R₃′″ are independently absent, a hydrogen, an unsubstituted aliphatic group, a substituted aliphatic group, an unsubstituted aliphatic ester group, a substituted aliphatic ester group, an unsubstituted aliphatic ether group, a substituted aliphatic ether group, an unsubstituted heterocyclic group, a substituted heterocyclic group, an unsubstituted heterocyclic-aliphatic group, or a substituted heterocyclic-aliphatic group;
(d) wherein R₈ and R₈′ are independently absent, a hydrogen, a halogen, a nitrile group, an ester group, an unsubstituted aliphatic ester group, a substituted aliphatic ester group, an unsubstituted amino group, a substituted amino group, an unsubstituted aliphatic amino group, a substituted aliphatic amino group, an amide group, a substituted aliphatic-amide group, an unsubstituted aliphatic-amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted aliphatic ether group, a substituted aliphatic ether group, un unsubstituted heterocyclic group, a substituted heterocyclic group, an unsubstituted heterocyclic-aliphatic group, or a substituted heterocyclic-aliphatic group; and
(e) wherein R₄, R₄′, R₅, R₈′, R₆, and R₇ are independently absent, a hydrogen, a halogen, a nitrile group, an oxo group, an ester group, an unsubstituted aliphatic ester group, a substituted aliphatic ester group, an unsubstituted amino group, a substituted amino group, an unsubstituted aliphatic-amino group, a substituted aliphatic-amino group, an amide group, an unsubstituted aliphatic-amide group, a substituted aliphatic-amide group, an azide group, an unsubstituted aliphatic group, a substituted aliphatic group, an unsubstituted aryl group, a substituted aryl group, an unsubstituted aryl-aliphatic group, a substituted aryl-aliphatic group, an unsubstituted aliphatic ether group, a substituted aliphatic ether group, un unsubstituted heterocyclic group, a substituted heterocyclic group, an unsubstituted heterocyclic-aliphatic group, or a substituted heterocyclic-aliphatic group;
(f) wherein the substituents are as defined in the base claim(s).

12. The compound of claim 11, wherein R₁ of Formulae 1-6; R₁′ of Formulae 7-24; R₂ of Formulae 86 and 88; R₂₂ of Formula 85; R₂₃ of Formulae 84, 87, and 96-102; R₂₅ of Formulae 92 and 95; and/or R₂₆ of Formulae 89-91, 93, and 94 are(is) an unsubstituted aliphatic ether or a substituted aliphatic ether, optionally an unsubstituted C₁-C₁₄ aliphatic ether or a substituted C₁-C₁₄ aliphatic ether, optionally an unsubstituted C₁-C₆ aliphatic ether or a substituted C₁-C₆ aliphatic ether.

13. The compound of claim 11, wherein R₁ of Formulae 1-6; R₁′ of Formulae 7-24; R₂ of Formulae 86 and 88; R₂₂ of Formula 85; R₂₃ of Formulae 84, 87, and 96-102; R₂₅ of Formulae 92 and 95; and/or R₂₆ of Formulae 89-91, 93, and 94 are(is) a substituted C₁-C₆ aliphatic ether having the structure of 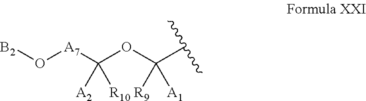
(a) wherein A₁ and A₂ are independently a hydrogen, a halogen, a hydroxyl group, a thiol group, a cyanide group, a nitrile group, an unsubstituted aliphatic group, a substituted aliphatic group, an unsubstituted heterocyclic group, a substituted heterocyclic group, an unsubstituted aryl group, or a substituted aryl group;
(b) wherein A₇ is a methylene group or an ethylene group;
(c) wherein R₉ and R₁₀ are independently a hydrogen, a hydroxyl group, an unsubstituted alkyl group, or a substituted alkyl group, optionally wherein R₉ and R₁₀ are independently a substituted alkyl group that together form a ring; and
(d) wherein B₂ is hydrogen or a phosphoramidate,
wherein the substituents are as defined in the base claim(s).

14. The compound of claim 13, wherein the substituted C₁-C₆ aliphatic ether has the structure of 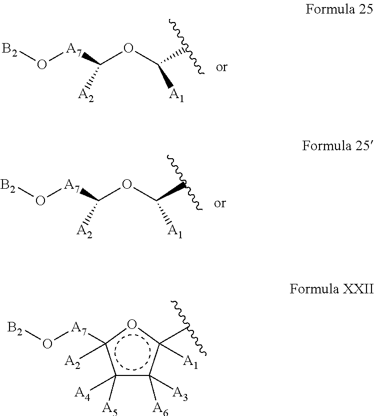
wherein A₃, A₄, A₅, and A₆ are independently absent, a hydrogen, a halogen, a hydroxyl group, a thiol group, an unsubstituted aliphatic group, a substituted aliphatic group, an unsubstituted heterocyclic group, a substituted heterocyclic group, an unsubstituted aryl group, or a substituted aryl group

15. The compound of claim 14, wherein the substituted C₁-C₆ aliphatic ether has the structure of 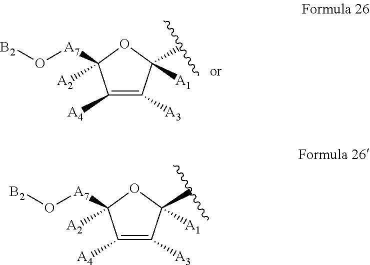
(a) wherein A₁ is an unsubstituted alkyl group, a substituted alkyl group, a cyanide group, a nitrile group, a hydroxyl group, or a halogen; and
(b) wherein A₂ is a hydrogen, a halogen, a hydroxyl group, a thiol group, an unsubstituted aliphatic group, a substituted aliphatic group, an unsubstituted heterocyclic group, a substituted heterocyclic group, an unsubstituted aryl group, or a substituted aryl group.

16. The compound of claim 14, wherein the substituted C₁-C₆ aliphatic ether has the structure of 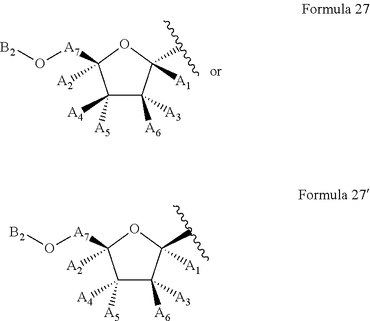
(a) wherein A₁ is an unsubstituted alkyl group, a substituted alkyl group, a cyanide group, a nitrile group, a hydroxyl group, or a halogen; and
(b) wherein A₂ is a hydrogen, a halogen, a hydroxyl group, a thiol group, an unsubstituted aliphatic group, a substituted aliphatic group, an unsubstituted heterocyclic group, a substituted heterocyclic group, an unsubstituted aryl group, or a substituted aryl group.

17. The compound of claim 14, wherein B₂ is a phosphoramidate having the structure of 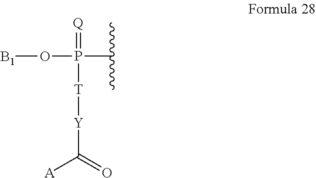
(a) wherein B₁ is a hydrogen, an unsubstituted aliphatic group, a substituted aliphatic group, an unsubstituted aryl group, or a substituted aryl group;
(b) wherein Q is an oxygen or a sulfur;
(c) wherein T is an oxygen, a sulfur, NR₁₁, CR₁₁R₁₂, CR₁₁W₁, or CW₁W₂, wherein R₁₁ and R₁₂ are independently a hydrogen, an unsubstituted alkyl group, a substituted alkyl group, an unsubstituted aryl group, a substituted aryl group, and wherein W₁ and W₂ are independently a heteroatom;
(d) wherein Y is an unsubstituted aliphatic group, a substituted aliphatic group, an unsubstituted heterocyclic group, a substituted heterocyclic group, an unsubstituted aryl group, or a substituted aryl group; and
(e) wherein A is an unsubstituted aliphatic group, a substituted aliphatic group, an unsubstituted aryl group, a substituted aryl group, an alkoxyl, —CM₁alkyl, —CM₁aryl, or —CM₁heterocyclic, wherein M₁ is an oxygen or a methylene group, optionally wherein A is —O-alkyl, where the alkyl can be a substituted or unsubstituted alkyl, such as an unsubstituted liner, branched, or cyclic C₁-C₆ alkyl, an unsubstituted liner, branched, or cyclic C₁-C₄ alkyl, or an unsubstituted liner, branched, or cyclic C₁-C₃ alkyl.

18. The compound of claim 11, wherein:
X is a hydrogen, an unsubstituted aliphatic group, a substituted aliphatic group, an unsubstituted aryl group, a substituted aryl group, an unsubstituted heterocyclic group, or a substituted heterocyclic group; or
R₃, R₃′, R₃″, R₃′″, R₈, and R₈′ are independently absent, a hydrogen, an unsubstituted aliphatic group, a substituted aliphatic group, an unsubstituted heterocyclic group, or a substituted heterocyclic group; or
R₄, R₄′, R₅, R₈′, R₆, and R₇ are independently absent, a hydrogen, an oxo group, an unsubstituted amino group, a substituted amino group, an unsubstituted aliphatic-amino group, a substituted aliphatic-amino group, an amide group, an unsubstituted aliphatic-amide group, a substituted aliphatic-amide group, an unsubstituted aliphatic group, a substituted aliphatic group, an unsubstituted aryl group, a substituted aryl group, an unsubstituted aryl-aliphatic group, a substituted aryl-aliphatic group, un unsubstituted heterocyclic group, a substituted heterocyclic group, an unsubstituted heterocyclic-aliphatic group, or a substituted heterocyclic-aliphatic group; or
a combination thereof.

19. A pharmaceutical composition or dosage unit comprising the compound of claim 1; and
a pharmaceutically acceptable excipient,
wherein the compound is in an effective amount to prevent, treat, or ameliorate one or more symptoms associated with a viral infection in a subject.

20. A method for preventing, treating, or ameliorating one or more symptoms associated with a viral infection in a subject comprising
(i) administering to the subject an effective amount of the compound of claim 1 to prevent, treat, or ameliorate one or more symptoms associated with the viral infection in the subject.
